(19) United States
(12) Patent Application Publication
(10) Pub. No.: US 2022/0162705 A1
(43) Pub. Date: May 26, 2022

(54) METHOD FOR PREDICTING THE RESPONSE TO CANCER IMMUNOTHERAPY IN CANCER PATIENTS

(71) Applicants: GBG FORSCHUNGS GMBH, Neu-lsenburg (DE); Charité - Universitätsmedizin Berlin, Berlin (DE)

(72) Inventors: Carsten DENKERT, Berlin (DE); Bruno SINN, Berlin (DE); Sibylle LOIBL, Neu-Isenburg (DE); Karsten WEBER, Dreieich (DE); Thomas KARN, Oberursel (DE)

(21) Appl. No.: 17/297,944

(22) PCT Filed: Nov. 29, 2019
(86) PCT No.: PCT/EP2019/083124

§ 371 Date: May 27, 2021

(30) Foreign Application Priority Data
Nov. 30, 2018 (EP) 18209672.7

(51) Int. Cl. C12Q 1/6886

(52) U.S. Cl. CPC C12Q 1/6886; C12Q 2600/158; C12Q 2600/106

(57) ABSTRACT

The present invention relates to methods, kits, systems and uses thereof for prediction of the response or resistance to and/or benefit from a cancer immunotherapy of a subject suffering from or being at risk of developing a neoplastic disease, in particular breast cancer, based on the measurement(s) of expression level(s) of at least one marker in samples of said subject. Equally, the present invention relates to methods, kits, systems and uses thereof for predicting the outcome from the cancer immunotherapy treatment in said subject based on the measurement(s) of the expression level(s) of the at least one marker in samples of said subject. Further, the present invention relates to the cancer immunotherapy for use in the treatment of the neoplastic disease, in particular breast cancer, in the subject and to methods for cancer immunotherapy treatment by using the cancer immunotherapy according to the methods of the present invention.

### FIELD OF INVENTION

The present invention relates to methods, kits, systems and uses thereof for prediction of the response or resistance to and/or benefit from a cancer immunotherapy of a subject suffering from or being at risk of developing a neoplastic disease, in particular breast cancer, based on the measurement(s) of expression level(s) of at least one marker in samples of said subject. Equally, the present invention relates to methods, kits, systems and uses thereof for predicting the outcome from the cancer immunotherapy treatment in said subject based on the measurement(s) of the expression level(s) of the at least one marker in samples of said subject.

### BACKGROUND OF THE INVENTION

In cancer therapy it is still a challenge to find the optimal therapy for a patient. For example, breast cancer is the most common neoplasia in women and remains one of the leading causes of cancer related deaths (Jemal et al., CA Cancer J Clin., 2013). Although the incidence has increased over years, the mortality has constantly decreased due to advances in early detection and development of novel effective treatment strategies. Breast cancer patients are frequently treated with radiotherapy, hormone therapy or cytotoxic chemotherapy prior to (neoadjuvant treatment) and/or after surgery (adjuvant treatment) to control for residual tumor cells and reduce the risk of recurrence.

A multitude of therapeutic treatment options are available and may include the combined use of several therapeutic agents, e.g. chemotherapeutic agents. For example, therapy can be applied in the neoadjuvant (preoperative) setting in which breast cancer patients receive systemic therapy before the remaining tumor cells are removed by surgery. In particular, systemic therapy is commonly applied to reduce the likelihood of recurrence in HER2/neu-positive and in tumors lacking the expression of the estrogen receptor and HER2/neu receptor (triple negative, basal).

According to today's therapy guidelines and current medical practice, the selection of a specific therapeutic intervention is mainly based on histology, grading, staging and hormonal status of the patient. In this regard, treatment decision concerning luminal, i.e. estrogen receptor positive and HER2/neu-negative, tumors are challenging since classical clinical factors like grading, tumor size or lymph node involvement do not provide a clear answer to the question whether to use chemotherapy or another therapeutic intervention or an additional therapeutic intervention. Thus, there is an urgent need for means and methods to predict the response to a particular treatment of a subject suffering from a neoplastic disease, in particular breast cancer, to reduce the number of patients suffering from serious side effects without clear benefit of the particular treatment and thus allow a more tailored treatment strategy. Another issue of lacking means and methods to predict the response to a particular treatment is the undertreatment of patients; one fourth of clinically high-risk patients suffer from distant metastasis during five years despite conventional cytotoxic chemotherapy. Those patients are undertreated and need additional or alternative therapies. Finally, one of the most open questions in current neoplastic diseases, in particular breast cancer therapy is which patients have a benefit from addition of further or alternative drugs, such as cancer immunotherapy, to conventional chemotherapy or other conventional non-chemotherapeutic interventions, such as hormone therapy. As such, there is a significant medical need to develop assays that identify patients that may respond and/or benefit from a cancer immunotherapy treatment in order to pinpoint therapeutic regimens tailored to the patient to assure optimal success. Currently, there are no reliable predictive biomarkers to identify the subgroup of patients who benefit from cancer immunotherapy treatment—preventing patient-tailored treatment.

Biomarkers can be analysed from pretherapeutic core biopsies to identify the most valuable predictive markers. For example, RNA may be isolated from core biopsies for the gene expression analysis. Based on the expression level data, which may be compared to a reference value, the therapeutic response may be directly evaluated. The therapeutic response of a particular tumor to the applied therapy may comprise the reduction of tumor mass in response to therapy or the pathological complete response (pCR) which refers to the complete eradication of cancer cells and lymph nodes after neoadjuvant treatment. However, in breast cancer patients, pCR is only observed in 10-25% of all patients. The pCR is an appropriate surrogate marker for disease a free survival and a strong indicator of benefit from chemotherapy. For patients with a low probability of response and/or benefit, other therapeutic approaches should be considered.

Specifically, multigene assays may provide superior or additional prognostic information to the standard clinical risk factors or analysis of a single biomarker. It is generally recognized, that proliferation markers seem to provide the dominant prognostic information. Unfortunately, until recently, there was no test in the market for prognosis or therapy prediction that come up with a more elaborated recommendation for the treating doctor whether and how to treat patients. Prominent examples of those predictors are the Mammaprint test from Agendia, the Relapse Score from Veridex and the Genomic Grade Index (GGI), developed at the institute Jules Bordet and licensed to Ipsogen. All of these assays are based on determination of the expression levels of at least 70 genes and all have been developed for RNA not heavily degraded by formalin fixation and paraffin embedding, but isolated from fresh tissue (shipped in RNALater™). For example, the GGI is a multigene test to define histologic grade of breast cancer based on gene expression profiles, in which a high GGI is associated with increased chemosensitivity in breast cancer patients treated with neoadjuvant therapy. Another prominent multigene assay is the Recurrence Score test of Genomic Health Inc. The test determines the expression level of 16 cancer related genes and 5 reference genes after RNA extraction from formalin fixed and paraffin embedded tissue samples. Although gene signatures have been shown to predict therapy response, the current tools suffer from a lack of clinical validity and utility including large-scale validation studies and clinical follow-up data, particularly in the most important clinical risk group, i.e. breast cancer patients of risk of recurrence based on standard clinical parameter. Therefore, none of these tools is commonly used to guide treatment decisions in clinical routine. Therefore, better tools are needed to optimize treatment decisions based on patient prognosis.

Examples of cancer immunotherapies include CAR T-cell therapies, cancer vaccines and immune checkpoint inhibitors. Immune checkpoint inhibitors that modulate cancer immunity have validated immunotherapy as a novel path to obtain durable and long-lasting clinical responses in cancer patients and are currently under research (Mellman et al., Nature, 2011, 480:480-489). The immune checkpoints are key regulators of the immune system that stimulate or inhibit its actions, which tumors can use to protect themselves from attacks by the immune system. Thus, immune checkpoint inhibitors are a type of drugs that block certain proteins made by some types of immune system cells, such as T cells, and some cancer cells. Hence, immune checkpoint inhibitors can block the inhibitory checkpoints, the so called “brakes” of the immune system, thereby releasing the “brakes” and restoring the immune system function, so that T cells are able to kill cancer cells better. Examples of checkpoint proteins found on T cells or cancer cells include PD-1/PD-L1 and CTLA-4/B7-1/B7-2. The first anti-cancer drug targeting an immune checkpoint was ipilimumab, a CTLA4 blocker approved in the United States in 2011.

Further immune checkpoint inhibitors under development are antibodies that block the interaction between the PD-1 receptor and its ligands PD-L1 and PD-L2 (Mullard, Nat. Rev. Drug Disc, 2013, 12:489-492). Several antibodies targeting the PD-1 pathway are currently in clinical development for treatment of melanoma, renal cell cancer, non-small cell lung cancer, diffuse large B cell lymphoma and other tumors.

Like many targeted therapies, responsiveness to immune checkpoint inhibitor treatment depends on a wide range of factors and is not uniform among patients; nonetheless, a fraction of all patients suffer significant adverse reactions to such treatment, e.g. Lipson et al, Clinical Cancer Research, 17(22): 6958-6962 (2011).

Hence, in view of the above, there is a continuing need of means and methods useful in making clinical decisions on the treatment and thus for advanced means and methods for the prediction of the response or resistance and/or benefit to and/or outcome from a cancer immunotherapy treatment of a subject suffering from or being at risk of developing a neoplastic disease.

Thus, the technical problem underlying the present invention is the provision of improved means and methods for predicting the response or resistance and/or benefit to and/or outcome of cancer immunotherapy treatment in a subject suffering from a neoplastic disease.

The present invention fulfills the continuing need for means and methods useful in making clinical decisions on the treatment and thus for advanced means and methods for the prediction of the response or resistance and/or benefit to and/or outcome from a cancer immunotherapy treatment of a subject suffering from or being at risk of developing a neoplastic disease on the basis of readily accessible clinical and experimental data.

The solution to this technical problem is provided by the embodiments as defined herein below and as characterized in the claims.

### BRIEF DESCRIPTION OF THE INVENTION

The present invention relates to a method for predicting a response or resistance to and/or a benefit from a cancer immunotherapy in a subject suffering from or being at risk of developing a neoplastic disease, in particular breast cancer, comprising the step of:

determining in a sample obtained from said subject the expression level of at least one marker selected from the group consisting of the markers as identified in Table 1 and/or Table 10.1,
wherein the expression level of the at least one marker is indicative for predicting the response or resistance to and/or the benefit from the treatment with the cancer immunotherapy in said subject.

Equally, the present invention relates to a method for predicting the outcome of a cancer immunotherapy in a subject suffering from or being at risk of developing a neoplastic disease, in particular breast cancer, comprising the step of:

determining in a sample obtained from said subject the expression level of at least one marker selected from the group consisting of the markers as identified in Table 1 and/or Table 10.1, wherein the expression level of the at least one marker is indicative for the outcome in said subject.

Equally, the present invention relates to a method for the prediction of the outcome in a subject suffering from or being at risk of developing a neoplastic disease, in particular breast cancer, wherein said subject receives a cancer immunotherapy, comprising the step of:

determining in a sample obtained from said subject the expression level of at least one marker selected from the group consisting of the markers as identified in Table 1 and/or Table 10.1,

In one aspect of the present invention, the expression level of at least one marker selected from the group consisting of the markers as identified in Table 2.1, Table 3.1, Table 4.1 and/or Table 5.1 is determined.

In one aspect of the present invention, the neoplastic disease is a recurrent neoplastic disease or a metastatic neoplastic disease or a non-metastatic disease, preferably the neoplastic disease is a non-metastatic disease.

In one aspect of the present invention, the neoplastic disease is a disease selected from the group consisting of breast cancer, lung cancer, renal cell carcinoma, melanoma, bladder cancer, urothelial carcinoma and Merkel-cell carcinoma, preferably breast cancer, more preferably the neoplastic disease is primary triple negative breast cancer (TNBC).

In one aspect of the present invention, the cancer immunotherapy is selected from the group consisting of immune checkpoint inhibitor therapy, chimeric antigen receptor (CAR) T-Cell therapy and cancer vaccine therapy, preferably the cancer immune therapy comprises treatment with an immune checkpoint inhibitor, even more preferably the immune checkpoint inhibitor is selected from the group consisting of a drug targeting CTLA4, a drug targeting PD-1 and a drug targeting PD-L1.

Herein, said cancer immunotherapy is preferably an immune checkpoint inhibitor therapy and the neoplastic disease is breast cancer.

In a preferred aspect of the present invention, the immune checkpoint inhibitor is a therapeutic antibody, more preferably the immune checkpoint inhibitor is an anti-CTLA4 antibody, an anti-PD-1 antibody or an anti-PD-L1 antibody and even more preferably the immune checkpoint inhibitor is selected from the group consisting of ipilimumab, nivolumab, pembrolizumab, atezolizumab, avelumab, durvalumab, cemiplimab, lambrolizumab, pidilizumab or a combination thereof.

In one aspect of the present invention, the sample of said subject is a formalin-fixed paraffin embedded sample or a fresh-frozen sample.

In one aspect of the present invention, the sample is a tumor sample or a lymph node sample obtained from said subject.

In one aspect of the present invention, the sample is an estrogen receptor negative and/or a HER2 negative sample.

In one aspect of the present invention, the expression level of the at least one marker is the protein expression level or the RNA expression level, preferably mRNA expression level. Preferably, the expression level is the RNA expression level, more preferably mRNA expression level, and is determined by at least one of a hybridization-based method, a PCR based method, a microarray-based method, a sequencing and/or next generation sequencing approach.

In one aspect of the present invention, the prediction of the response, resistance, benefit and/or outcome is for a combination of the cancer immunotherapy with a non-chemotherapy or a chemotherapy, preferably a neoadjuvant therapy. Preferably the non-chemotherapy or the chemotherapy is concomitant with and/or sequential to the cancer immunotherapy.

In one aspect of the present invention, the method is a method for therapy monitoring.

In one aspect of the present invention, the response, resistance, benefit and/or outcome to be predicted is at least 12 weeks, at least 14 weeks, at least 20 weeks, at least 22 weeks, after the start of the cancer immunotherapy treatment, more preferably after surgery.

In one aspect of the present invention, the response or resistance and/or benefit and/or outcome is the pathological complete response (pCR), loco-regional recurrence free interval (LRRFI), loco-regional invasive recurrence free interval (LRIRFI), distant-disease-free survival (DDFS), invasive disease-free survival (IDFS), event free survival (EFS) and/or overall survival (OS).

In one aspect of the present invention, the method comprises comparing the expression level of each of said at least one marker to a predetermined reference level.

In one aspect of the present invention, the reference level comprises the expression level of the at least one marker in a sample obtained from at least one healthy subject, preferably the mean expression level of the at least one marker in samples obtained from a healthy population.

In one aspect of the present invention, the method further comprises the determination of one or more clinical parameters selected from the group consisting of pathological grading of the tumor, tumor size and nodal status.

In one aspect of the present invention, in said sample obtained from said subject the expression levels of at least two, at least three, at least four, at least five, at least ten, at least twenty markers selected from the group consisting of the markers as identified in Table 6.1, Table 7, Table 8.1, Table 2.1, Table 3.1, Table 4.1, Table 5.1 and Table 10.1 are determined.

In a preferred aspect of the present invention, the method comprises determining a score based on
(i) the expression levels of the at least two, at least three, at least four, at least five, at least ten, at least twenty markers; or
(ii) the expression level of the at least one marker and the at least one clinical parameter.

In one aspect of the present invention,
(a) the at least one marker is selected from the group of the markers as identified in Table 2.1, preferably in Table 2.2, more preferably in Table 2.3, more preferably in Table 2.4, more preferably in Table 2.5, more preferably in Table 2.6, more preferably in Table 2.7, more preferably in Table 2.8, more preferably in Table 2.9, more preferably in Table 2.10, more preferably in Table 2.11 and even more preferably in Table 2.12; and/or
(b) the at least one marker is selected from the group of the markers as identified in Table 3.1, preferably in Table 3.2, more preferably in Table 3.3, more preferably in Table 3.4, more preferably in Table 3.5, more preferably in Table 3.6, more preferably in Table 3.7, more preferably in Table 3.8, more preferably in Table 3.9, more preferably in Table 3.10, more preferably in Table 3.11 and even more preferably in Table 3.12; and/or
(c) the at least one marker is selected from the group of the markers as identified in Table 4.1, preferably in Table 4.2, more preferably in Table 4.3, more preferably in Table 4.4, more preferably in Table 4.5, more preferably in Table 4.6, more preferably in Table 4.7, more preferably in Table 4.8, more preferably in Table 4.9, more preferably in Table 4.10, more preferably in Table 4.11 and even more preferably in Table 4.12; and/or
(d) the at least one marker is selected from the group of the markers as identified in Table 5.1, preferably in Table 5.2, more preferably in Table 5.3, more preferably in Table 5.4, more preferably in Table 5.5, more preferably in Table 5.6, more preferably in Table 5.7, more preferably in Table 5.8, more preferably in Table 5.9, more preferably in Table 5.10, more preferably in Table 5.11 and even more preferably in Table 5.12; and/or
(e) the at least one marker is selected from the group of the markers as identified in Table 6.1, preferably in Table 6.2, more preferably in Table 6.3, more preferably in Table 6.4, more preferably in Table 6.5, more preferably in Table 6.6, more preferably in Table 6.7, more preferably in Table 6.8, more preferably in Table 6.9, more preferably in Table 6.10, more preferably in Table 6.11 and even more preferably in Table 6.12; and/or
(f) the at least one marker is selected from the group of the markers as identified in Table 7; and/or
(g) the at least one marker is selected from the group of the markers as identified in Table 8.1, preferably in Table 8.2, more preferably in Table 8.3, more preferably in Table 8.4, more preferably in Table 8.5, more preferably in Table 8.6, more preferably in Table 8.7, more preferably in Table 8.8, more preferably in Table 8.9, more preferably in Table 8.10, more preferably in Table 8.11 and even more preferably in Table 8.12.

Further the present invention relates to a cancer immunotherapy for use in the treatment of a neoplastic disease, wherein the cancer immunotherapy treatment is administered to a subject that has been identified to respond to said treatment or that has been identified to benefit from said treatment or for whom said treatment has been determined to have a positive outcome according to the method of the present invention.

In one aspect of the present invention, the treatment comprises a combination of the cancer immunotherapy treatment with a non-chemotherapy treatment and/or a chemotherapy, preferably a neoadjuvant therapy. Preferably, the chemotherapy comprises one or more of the chemotherapeutic agent(s) selected from the group consisting of paclitaxel and nab-paclitaxel. Preferably, the non-chemotherapy comprises one or more of the group consisting of surgery, hormone therapy, radiation therapy, targeted therapy, poly ADP ribose polymerase (PARP) inhibitor therapy, cyclin dependent kinase (CDK) inhibitor therapy, such as CDK4/6 inhibitor therapy and combinations thereof.

Further, the present invention relates to the use of the method according to the method of the present invention for therapy control, therapy guidance, monitoring, risk assessment, and/or risk stratification in a subject suffering from or being at risk of developing a neoplastic disease.

Further, the present invention relates to a method of treating a subject suffering from a neopalstic disease or being at risk of developing a neoplastic disease with a cancer immunotherpay, wherein the subject to be treated with a cancer immunotherapy is a subject that has been predicted to respond and/or to benefit from the treatment with the cancer immunotherapy and/or has been predicted with a positive outcome with treatment with the cancer immunotherapy according to the methods of the present invention.

In one aspect of the present invention, the treatment comprises a combination of the cancer immunotherapy treatment with a non-chemotherapy and/or a chemotherapy, preferably a neoadjuvant therapy. Preferably, the chemotherapy comprises one or more of the chemotherapeutic agent(s) selected from the group consisting of paclitaxel and nab-paclitaxel. Preferably, the non-chemotherapy comprises one or more of the group consisting of surgery, hormone therapy, radiation therapy, targeted therapy, poly ADP ribose polymerase (PARP) inhibitor therapy, cyclin dependent kinase (CDK) inhibitor therapy, such as CDK4/6 inhibitor therapy and combinations thereof.

### FIGURES

FIG. 1: Study design of a randomised, double-blind, multi-centre phase II trial to assess the pathological complete response rate in the case of neoadjuvant therapy with sequentially administered nab-paclitaxel followed by EC+/−PD-L1 antibody MED14736 (i.e. durvalumab) in patients with early-stage breast cancer (TNBC). Durvalumab or placebo was given every 4 weeks (in addition to nab-paclitaxel followed by standard EC). Some patients participated in the window phase, wherein durvalumab/placebo alone was given two weeks prior to start of nab-paclitaxel followed by a biopsy.

### DETAILED DESCRIPTION OF THE INVENTION

Unless defined otherwise, technical and scientific terms used herein have the same meaning as commonly understood by one of ordinary skill in the art to which this invention belongs.

The present invention relates to a method for predicting a response or resistance to and/or a benefit from a cancer immunotherapy in a subject suffering from or being at risk of developing a neoplastic disease, in particular breast cancer, comprising the step of:

determining in a sample obtained from said subject the expression level of at least one marker selected from the group consisting of the markers as identified in Table 1 and/or Table 10.1, wherein the expression level of the at least one marker is indicative for predicting the response or resistance to and/or the benefit from the treatment with the cancer immunotherapy in said subject.

For example, such a marker may refer to a marker selected from the group consisting of PTPN11, DIABLO, PARP2, MTHFD1, MAX, HERPUD1, RAD51C, P4HB, PYCR1, SPOP, PHB, XRCC5, PPP2CB, MYBL1, STK3, TNFRSF17, CD79A, COL9A3, PLA2G4A, SPRY2, KCNK5, DMD, DDX58, ISG15, IFI27, MX1, IRF9, IRF7, CXCL1, CXCL8, CCL19, CCL7, LAG3, THBS4, PTPRC, ITGB7, PRDM1, TNFRSF9, CD86, CXCL13, CXCL16, STAT1, IDO1, GBP1, IRF1, TAP1, CXCL10, KRT7, KRT18, DLGAP5, MCM6, FBXO5, E2F3, EZH2, FANCG, TTK, KDM1A, MCM5, GMPS, NASP, SMC4, MAD2L1, KNTC1, PRC1, CDCl₇, TK1, CCNE2, BLM, COL3A1, MMP14, SFRP2, COL5A1, COL1A2, COL1A1, FN1, LOXL1, PCOLCE, COL5A2, SPARC, IGFBP7, THBS2, SFRP4, VCAN, CD38, GNLY, GZMB, SLAMF7, CD8A, IRF4 and CCL5, preferably DDX58, LAG3, THBS4, COL3A1, COL1A1, CD38, GNLY, IFI27, MX1, IRF9, IRF7, CXCL13, STAT1, GBP1, IRF1, TAP1, CXCL10, KDM1A, KNTC1, SPARC, IGFBP7, SLAMF7, RAD51C, P4HB, MYBL1, PLA2G4A, CCL19, CCL7, KRT7, MMP14, SFRP2, COL5A1 and COL1A2,

most preferably DDX58, LAG3, THBS4, COL3A1, COL1A1, CD38 and GNLY.

As another example, such a marker may refer to a marker selected from the group consisting of DDX58, IFI27, MX1, IRF9, IRF7, LAG3, THBS4, CXCL13, STAT1, GBP1, IRF1, TAP1, CXCL10, KDM1A, KNTC1, COL3A1, COL1A1, SPARC, IGFBP7, CD38, GNLY and SLAMF7, wherein the expression level of the at least one marker is indicative for predicting the response or resistance to and/or the benefit from the treatment with the cancer immunotherapy in said subject.

As still another example, such a marker may refer to a marker selected from the group consisting of RAD51C, P4HB, MYBL1, PLA2G4A, DDX58, CCL19, CCL7, LAG3, THBS4, KRT7, COL3A1, MMP14, SFRP2, COL5A1, COL1A2, COL1A1, CD38 and GNLY, wherein the expression level of the at least one marker is indicative for predicting the response or resistance to and/or the benefit from the treatment with the cancer immunotherapy in said subject.

In another aspect, the present invention relates to a method for predicting a response or resistance to and/or a benefit from a cancer immunotherapy in a subject suffering from or being at risk of developing a neoplastic disease, in particular breast cancer, comprising the step of:

determining in a sample obtained from said subject the expression level of at least one marker selected from the group consisting of GNLY, GZMB, CD8A, CCL5, CD38, IRF4, SLAMF7, CXCL1, CA9, PRF1, APOL3, CCR5, CXCR6, CDCl₃D, IL2RG, IL2RB, GZMA, FGL2, CD27, CXCR3, CXCL2, CXCL3, CXCL5, CXCL8, BNIP3, HK2, NDRG1, ADM, ANGPTL4 and SLC2A1, wherein the expression level of the at least one marker is indicative for predicting the response or resistance to and/or the benefit from the treatment with the cancer immunotherapy in said subject.

In one preferred aspect, the invention relates to a method for predicting a response or resistance to and/or a benefit from a cancer immunotherapy in a subject suffering from or being at risk of developing a neoplastic disease, in particular breast cancer, comprising the steps of:

determining in a sample obtained from said subject the expression level of at least one marker related to immune response and/or a marker related to antigen-presentation of a tumor cell, wherein the expression level of the at least one marker is indicative for predicting the response or resistance to and/or the benefit from the cancer immunotherapy in said subject.

In one preferred aspect, the invention relates to a method for predicting the outcome of a cancer immunotherapy treatment in a subject suffering from or being at risk of developing a neoplastic disease, in particular breast cancer, comprising the step of:

determining in a sample obtained from said subject the expression level of at least one marker related to immune response and/or a marker related to antigen-presentation of a tumor cell, wherein the expression level of the at least one marker is indicative for the outcome in said subject.

In one preferred aspect, the invention relates to a method for the prediction of the outcome in a subject suffering from or being at risk of developing a neoplastic disease, in particular breast cancer, wherein said subject is treated with a cancer immunotherapy, comprising the step of:

determining in a sample obtained from said subject the expression level of at least one marker related to immune response and/or a marker related to antigen-presentation of a tumor cell, wherein the expression level of the at least one marker is indicative for the outcome in said subject.

Said at least one marker related to immune response and/or a marker related to antigen-presentation of a tumor cell may herein in particular refer to a marker selected from the group consisting of CCL19, CCL7, LAG3, THBS4, PTPRC, ITGB7, PRDM1, TNFRSF9, CD86, CXCL13, CXCL16, STAT1, IDO1, GBP1, IRF1, TAP1, CXCL10, APOL3, CCR5, CXCR6, CD3D, IL2RG, IL2RB, GZMA, FGL2, PRF1, CD27, CXCR3, CD38, GNLY, GZMB, SLAMF7, CD8A, IRF4, CCL5, CXCL1, CXCL2, CXCL3, CXCL5 and CXCL8.

In one aspect, the marker is a marker related to related to immune response selected from the group consisting of CCL19, CCL7, LAG3, THBS4, PTPRC, ITGB7, PRDM1, TNFRSF9, CD86, CXCL13 and CXCL16, preferably CCL19, CCL7, LAG3, THBS4, TNFRSF9, CD86 and CXCL13, most preferably CCL19, CCL7, LAG3, THBS4 and CXCL13.

In one aspect, the marker is a marker related to antigen-presentation of a tumor cell selected from the group consisting of APOL3, CCR5, CXCR6, CD3D, IL2RG, IL2RB, GZMA, FGL2, PRF1, CD27, CXCR3, CD38, GNLY, GZMB, SLAMF7, CD8A, IRF4 and CCL5, preferably selected from the group consisting of CD38, GNLY, GZMB, SLAMF7, CD8A, IRF4 and CCL5, most preferably said maker is GNLY or GZMB.

In one aspect, the invention relates to a method for predicting a response or resistance to and/or a benefit from a cancer immunotherapy in a subject suffering from or being at risk of developing a neoplastic disease, in particular breast cancer, comprising the steps of:

determining in a sample obtained from said subject the expression level of at least one marker related to the VEGFA-mediated signaling pathway, wherein the expression level of the at least one marker is indicative for predicting the response or resistance to and/or the benefit from the cancer immunotherapy in said subject.

In one aspect, the invention relates to a method for predicting the outcome of a cancer immunotherapy treatment in a subject suffering from or being at risk of developing a neoplastic disease, in particular breast cancer, comprising the step of:

determining in a sample obtained from said subject the expression level of at least one marker related to the VEGFA-mediated signaling pathway, wherein the expression level of the at least one marker is indicative for the outcome in said subject.

In one aspect, the invention relates to a method for the prediction of the outcome in a subject suffering from or being at risk of developing a neoplastic disease, in particular breast cancer, wherein said subject is treated with a cancer immunotherapy, comprising the step of:

determining in a sample obtained from said subject the expression level of at least one marker related to the VEGFA-mediated signaling pathway, wherein the expression level of the at least one marker is indicative for the outcome in said subject.

Herein, the marker related to the VEGFA-mediated signaling pathway may in particular be selected from the group consisting of BNIP3, HK2, CA9, NDRG1, ADM, ANGPTL4, SLC2A1 and VEGFA.

The present invention relates to a method for predicting a response or resistance to and/or a benefit from a cancer immunotherapy in a subject suffering from or being at risk of developing a neoplastic disease, in particular breast cancer, comprising the step of:
determining in a sample obtained from said subject the expression level of at least one marker selected from the group consisting of the markers as identified in Table 1 and Table 10.1.

TABLE 1
ACKR2, ACSL3, ACSL4, ACSL5, ACTA2, ACTR3B, ADAMTS1, ADIPOR1, AGT, AHNAK,
AK3, AKT2, ALDH1A3, ALDOC, ALKBH3, ANGPT1, APAF1, AR, AREG, ARID1A, ARNT,
ATP5F1, ATP6V0C, ATP6V1G2, BATF, BCE10, BCE2A1, BID, BIRC7, BEM, BMP5, BOK,
C5orf55, CA9, CAD, CASP8AP2, CAV1, CAV2, CBX3, CCDC103, CCE14, CCE17, CCE18,
CCE19, CCE21, CCE22, CCE25, CCE28, CCE3, CCE4, CCE5, CCE7, CCND3, CCNE2, CCR4,
CCT4, CCT6B, CD274, CD38, CD47, CD55, CD79A, CD83, CD86, CD8A, CDC7, CDKN2A,
CDX2, CEACAM3, CEBPB, CELSR2, CHI3E1, CHMP4B, CECF1, CMKLR1, COE1A1, COE1A2,
COE2A1, COE3A1, COE5A1, COE5A2, COE9A3, COX7B, CRK, CREF2, CRY1, CSDE1,
CXCE1, CXCE10, CXCE13, CXCE16, CXCE8, CXXC4, CYP4V2, DAAM1, DDX58, DHX58,
DIABLO, DLC1, DLGAP5, DLL4, DMD, DNAJA1, DNAJB2, DNAJC10, DNAJC13, DNAJC14,
DNAJC8, DUSP6, E2F3, EAF2, EDIL3, EEF2K, EGER, EIF6, ENG, EPCAM, ER_154, ERBB2,
ETV7, EZH2, FABP4, FADD, FAF1, FANCG, FAS, FASN, FBXO5, FBXW11, FGF13, FGF4,
FGFR3, FLT3, FN1, FOSL1, GADD45G, GBP1, GBP7, GJA1, GLIS3, GMPS, GNG12, GNLY,
GPAM, GPAT2, GPR17, GRIN2A, GSN, GSR, GSTM1, GZMB, HDAC8, HERPUD1, HEY2,
HIC1, HIST1H3H, HLA_A, HLA_B, HLA_E, HMGB3, HMOX1, HRK, HSPA1A, HSPA1L, ID1,
ID2, IDH1, IDH2, IDO1, IFI27, IFNA2, IFNA5, IFNAR1, IFNW1, IGFBP7, IL12A, IL6R, INHBA,
IRF1, IRF2, IRF4, IRF7, IRF9, IRS1, ISG15, ITGA2, ITGB7, ITPKB, JAG1, JAK1, JAK2, JPH3,
KCNK5, KDM1A, KDM6A, KDR, KIF3B, KNTC1, KRT18, KRT7, LAG3, LCN2, LFNG, LIF,
LOX, LOXL1, LRIG1, LRP12, LYVE1, MAD2L1, MADD, MAP3K4, MAP3K5, MAPK10,
MAPK3, MAT2A, MAX, MCM5, MCM6, MED12, MESP1, MGEA5, MIXL1, MLLT3, MLPH,
MME, MMP14, MSH3, MSL2, MTHFD1, MX1, MYBL1, MYCN, MYOD1, NAIP, NAMPT,
NASP, NCOA2, NFKB1, NKD1, NLRP3, NMU, NOD2, NOTCH1, NOTCH4, NR6A1, NRG1,
NSD1, NTHL1, NTRK1, NUMBL, ORM2, P4HB, PAG1, PARP2, PAX6, PCOLCE, PDCD1LG2,
PDGFB, PFKFB3, PHB, PIK3CA, PIM3, PLA2G10, PLA2G4A, PLAT, PLCB1, PLCG1, PLCG2,
PLK4, PMEPA1, PML, PPARGC1A, PPID, PPP2CA, PPP2CB, PRAME, PRC1, PRDM1,
PRKAA2, PRKAG1, PRKCE, PRMT6, PROMI, PRR15L, PSIP1, PSMD2, PTCHD1, PTGR1,
PTP4A1, PTPN11, PTPRC, PTTG1, PYCR1, QSOX2, RAB6B, RAC3, RAD51C, RAD9A, RARB,
RASSF1, RBI, RBP1, RELN, RIPK3, RPL13, RPL6, RUNX1, S100A6, SCUBE2, SELE,
SERPINB2, SERPINF1, SETD2, SFRP2, SFRP4, SHC2, SLAMF7, SLC11A1, SLC16A1,
SLC16A2, SLC25A13, SLC45A3, SLIT2, SMAD2, SMC1A, SMC4, SNCA, SOCS4, SORT1,
SPARC, SPDEF, SPINK1, SPOP, SPRY2, SPRY4, SRF, SRM, STAT1, STEAP4, STK3, STK39,
STX1A, TADA3, TAP1, TAP2, TBL1X, TBL1Y, TERF1, TGFBR2, THBS2, THBS4, TIFA,
TIMP3, TK1, TLR3, TMEM45B, TMEM74B, TNFAIP3, TNFRSF11B, TNFRSF17, TNFRSF8,
TNFRSF9, TNFSF14, TNXB, TOP1, TOP3A, TSPAN13, TSPAN7, TTK, UBB, UBXN2A,
UGT1A1, USF2, VCAN, VEGFB, VGLL4, VHL, WNT10A, WNT7B, WWC1, WWOX, XBP1,
XRCC5, ZAK

TABLES 10.1 AND 10.2

| | |
|---|---|
| 10.1 | CASP4, LRRK2, GGH, C3AR1, ARMC1, FANCC, MAF, RASA1, PIAS1, HERC3, |
| | SLA, CFLAR, RUNX2, FAF1, CTLA4, TNFSF14, MAPKAPK5, LAMA5, PTEN, |
| | BID, FYN, E2F3, ALDH1A1, PDPN, NOX4, MYBL2, RBP1, SYCP2 |
| 10.2 | CASP4, LRRK2, GGH, C3AR1, ARMC1, FANCC, MAF, RASA1, PIAS1, HERC3, |
| | SLA, CFLAR, RUNX2, CTLA4, MAPKAPK5, LAMA5, PTEN, FYN, ALDH1A1, |
| | PDPN, NOX4, MYBL2, SYCP2 |

wherein the expression level of the at least one marker is indicative for predicting the response or resistance to and/or the benefit from the cancer immunotherapy.

Equally, the invention relates to the use of the method of the present invention.

Equally, the invention relates to a cancer immunotherapy for use in the treatment of a neoplastic disease, wherein the cancer immunotherapy is administered to a subject that has been identified to respond to said treatment or that has been identified to benefit from said treatment or for whom said treatment has been determined to have a positive outcome according to the method of the present invention.

Equally, the invention relates to a method of treating a subject suffering from a neopalstic disease or being at risk of developing a neoplastic disease with a cancer immunotherapy, wherein the subject to be treated with the cancer immunotherapy is a subject that has been predicted to respond and/or to benefit from the treatment with the cancer immunotherapy and/or has been prognosticated with a positive outcome with treatment with the cancer immunotherapy according to the method of the present invention.

As used herein, the term “prediction” relates to an individual assessment of the malignancy of a tumor or to the expected survival rate (OS, overall survival or DFS, disease free survival) of a patient undergoing a given therapy, i.e. treatment with a cancer immunotherapy, and of the patient who is not treated, i.e. no treatment with the cancer immunotherapy. In other words, the term “prediction” refers to the comparison of the response or the resistance to and/or benefit to (i) a treatment with a cancer immunotherapy to (ii) a treatment without the cancer immunotherapy. The subject may be treated with further other components, such as chemotherapeutic agents and/or non-chemotherapeutic agents in both groups. A predictive marker relates to a marker which can be used to predict the response or resistance and/or benefit of the subject towards a given treatment, e.g. the treatment with a cancer immunotherapy. As used herein, the term “predicting the response to a treatment with a cancer immunotherapy” refers to the act of determining a likely response or resistance and/or benefit of the treatment with the cancer immunotherapy in a subject suffering from or being at risk of developing a neoplastic disease. The prediction of a response or resistance and/or benefit is preferably made with reference to a reference value described below in detail. The predictive methods of the present invention can be used clinically to make treatment decisions by choosing the most appropriate treatment modalities for the subject.

As used herein, the terms “predicting an outcome” and “prediction of an outcome” of a disease are used interchangeably and refer to a prediction of an outcome of a patient undergoing a given therapy, i.e. treatment with a cancer immunotherapy. The terms “predicting an outcome” and “prediction of an outcome” may, in particular, relate to an individual assessment of the malignancy of a tumor, or to the expected survival rate (OS, overall survival or DFS, disease free survival) of a patient, if the tumor is treated with a given therapy, i.e. the treatment with a cancer immunotherapy.

As used herein, the term “predicting a resistance to a cancer immunotherapy” relates to a prediction of a resistance of a patient undergoing a given therapy, i.e. treatment with a cancer immunotherapy. The term “predicting a resistance to a cancer immunotherapy” may, in particular, relate to a non-response and/or a non-benefit in said subject by individual assessment of the malignancy of a tumor, or to the expected survival rate (OS, overall survival or DFS, disease free survival) of a patient, if the tumor is treated with a given therapy, i.e. the treatment with a cancer immunotherapy.

As used herein, the term “treatment”, “treat”, “treating” and grammatical variations thereof refer to subjecting an individual subject to a protocol, regimen, process or remedy, in which it is desired to obtain a physiologic response or outcome in that subject, e.g., a patient. In particular, the methods and compositions of the present invention may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population may fail to respond or respond inadequately to treatment.

As used herein, the term “disease” is defined as a deviation from the normal structure or function of any part, organ or system of the body (or any combination thereof). A specific disease is manifested by characteristic symptoms and signs, including both chemical and physical changes. Certain characteristic signs, symptoms, and related factors of the disease can be quantitated through a variety of methods to yield important diagnostic information. For example, the neoplastic disease may be a tumor or cancer. As used herein, the term “tumor” refers to all neoplastic cell growth and proliferation, whether malignant or benign, and all pre-cancerous and cancerous cells and tissues. As used herein, the term “cancer” refers to uncontrolled cellular growth, and is not limited to any stage, grade, histomorphological feature, invasiveness, agressivity, or malignancy of an affected tissue or cell aggregation. For example, stage 0 breast cancer, stage I breast cancer, stage II breast cancer, stage III breast cancer, stage IV breast cancer, grade I breast cancer, grade II breast cancer, grade III breast cancer, malignant breast cancer, primary carcinomas of the breast, and all other types of cancers, malignancies and transformations associated with the breast are included. As used herein, the term “neoplastic lesion” or “neoplastic disease” or “neoplasia” refers to a cancerous tissue this includes carcinomas, (e.g., carcinoma in situ, invasive carcinoma, metastatic carcinoma) and pre-malignant conditions, neomorphic changes independent of their histological origin (e.g. ductal, lobular, medullary, mixed origin).

In one embodiment, the expression level of at least one marker selected from the group consisting of the markers as identified in Table 2.1, Table 3.1, Table 4.1 and Table 5.1

TABLES 2.1 TO 2.12
Table 2.1
ACSL4, AKT2, BCL2A1, BLM, BTK, CA9, CASP8AP2, CCL5, CCL7, CCNA2, CCR2, CD27,
CD274, CD38, CD79A, CD83, CDKN2A, CXCL10, CXCL13, CXCR3, CYBB, CYP3A4, DDX58,
DHX58, DLGAP5, DMD, DNAJB7, DNAJC14, ETV7, FGF14, FGL2, GBP1, GNLY, GSTA2,
GZMB, HERPUD1, HIST1H3H, HLA_A, HLA_B, HLA_E, IFIT2, IFNA2, IFNA5, IL10RA,
IL12A, IL17F, IL2, IL2RB, IL2RG, IL6R, IRF2, IRF4, IRF7, IRF9, ISG15, JAK2, KDM1A,
KNTC1, LAG3, MAD2L1, MAPK10, MCM6, MLLT3, MSL2, MTHFD1, MX1, OAS1,
PDCD1LG2, PIM2, PLK4, PML, PRF1, PSIP1, RAB6B, RSPO2, SCN3A, SLAMF7, SLC22A2,
SOCS4, SRM, STAT1, TAP1, TAP2, TBL1X, TIFA, TLR3, TNFRSF17
Table 2.2
ACSL4, AKT2, BCL2A1, BLM, CA9, CASP8AP2, CCL7, CD274, CD38, CD83, CDKN2A,
CXCL10, CXCL13, DDX58, DHX58, DLGAP5, DMD, DNAJC14, ETV7, GBP1, GNLY,
HERPUD1, HIST1H3H, HLA_A, HLA_B, IFNA2, IL12A, IL6R, IRF2, IRF4, IRF7, IRF9, JAK2,
KDM1A, KNTC1, LAG3, MAD2L1, MAPK10, MCM6, MLLT3, MSL2, MTHFD1, MX1,
PDCD1LG2, PLK4, PML, PSIP1, RAB6B, SLAMF7, SOCS4, SRM, STAT1, TAP1, TAP2, TBL1X,
TIFA, TLR3, TNFRSF17
Table 2.3
AKT2, BTK, CA9, CCL5, CCR2, CD27, CD274, CD38, CD79A, CDKN2A, CXCL10, CYBB,
CYP3A4, DMD, DNAJB7, ETV7, FGF14, GBP1, GNLY, GSTA2, GZMB, HERPUD1, HLA_A,
HLA_B, HLA_E, IFNA2, IFNA5, IL10RA, IL17F, IL2, IL2RB, IL2RG, IL6R, IRF2, IRF4, IRF7,
JAK2, KDM1A, KNTC1, LAG3, MAPK10, MCM6, MLLT3, MSL2, PDCD1LG2, PIM2, PRF1,
PSIP1, RSPO2, SCN3A, SLAMF7, SLC22A2, SOCS4, STAT1, TAP1, TAP2, TBL1X, TIFA,
TNFRSF17
Table 2.4
AKT2, CA9, CD274, CD38, CDKN2A, CXCL10, DMD, ETV7, GBP1, GNLY, HERPUD1,
HLA_A, HLA_B, IFNA2, IL6R, IRF2, IRF7, JAK2, KDM1A, KNTC1, LAG3, MAPK10, MCM6,
MLLT3, MSL2, PDCD1LG2, PSIP1, SOCS4, STAT1, TAP1, TAP2, TBL1X, TIFA, TNFRSF17
Table 2.5
AKT2, CCL5, CD27, CD274, CD38, CDKN2A, DMD, ETV7, GBP1, GNLY, GZMB, HERPUD1,
HLA_A, HLA_B, HLA_E, IL10RA, IL2RB, IL2RG, IL6R, IRF4, IRF7, LAG3, MLLT3, PIM2,
PRF1, PSIP1, SLAMF7, SOCS4, STAT1, TAP1, TBL1X, TIFA
Table 2.6
AKT2, CD274, CD38, CDKN2A, DMD, ETV7, GBP1, GNLY, HERPUD1, HLA_A, HLA_B, IL6R,
IRF7, LAG3, MLLT3, PSIP1, SOCS4, TAP1, TBL1X, TIFA
Table 2.7
AKT2, CCL5, CD27, CD38, ETV7, GBP1, GNLY, GZMB, HERPUD1, HLA_A, HLA_B, HLA_E,
IL10RA, IL2RB, IL2RG, IL6R, IRF4, PIM2, PRF1, PSIP1, SLAMF7, SOCS4, STAT1, TAP1, TIFA
Table 2.8
AKT2, CD38, ETV7, GNLY, HERPUD1, HLA_B, IL6R, PSIP1, SOCS4, TAP1, TIFA
Table 2.9
CCL5, CD27, CD38, ETV7, GBP1, GNLY, GZMB, HERPUD1, IL10RA, IL2RB, IL2RG, IRF4,
PIM2, PRF1, PSIP1, SLAMF7, SOCS4, STAT1, TAP1
Table 2.10
CD38, ETV7, GNLY, HERPUD1, PSIP1, SOCS4, TAP1
Table 2.11
CCL5, ETV7, GBP1, GNLY, GZMB, HERPUD1, IL2RB, PRF1, PSIP1, SOCS4, STAT1, TAP1
Table 2.12
ETV7, GNLY, HERPUD1, PSIP1, SOCS4, TAP1

TABLES 3.1 TO 3.12
Table 3.1
ACKR1, ACTA2, ACTB, AHNAK, BATF, BCL10, BMP5, BOK, CALML6, CAV1, CAV2,
CCL14, CCL17, CD55, CHMP4B, CLCF1, CMKLR1, COL11A1, COL1A1, COL1A2, COL3A1,
COL5A1, COL5A2, CRY1, DLL4, DNAJB14, DNAJB2, DNAJB8, EDIL3, EGFR, ENG, ER_013,
ER_028, ER_067, FBN1, FGF13, FN1, GSN, GSR, HEY2, HIC1, HSPA9, IGFBP7, IL13, INHBA,
IRS1, ITGA2, JAG1, KDR, LFNG, LOX, LRP12, MED12, MFNG, MMP2, MMS19, NOTCH1,
NOTCH4, PAG1, PDGFB, PIM3, PLAT, PMEPA1, PPP2CB, PRKCE, PRMT6, RAC3, RB1,
RIPK3, RUNX1, S100A6, SERPINF1, SFRP2, SHC2, SLC22A6, SLC25A13, SLIT2, SNCA,
SPARC, SPRY4, SRF, STK3, STK39, TBL1Y, THBS2, THBS4, TIE1, TIMP2, TIMP3, TMEM74B,
TNFRSF11B, TNFSF14, TNXB, TRIB1, VEGFB, YY1
Table 3.2
ACTA2, AHNAK, BATF, BCL10, BMP5, BOK, CAV1, CAV2, CCL14, CCL17, CD55, CHMP4B,
CLCF1, CMKLR1, COL1A1, COL1A2, COL3A1, COL5A1, COL5A2, CRY1, DLL4, DNAJB2,
EDIL3, EGFR, ENG, FGF13, FN1, GSN, GSR, HEY2, HIC1, IGFBP7, INHBA, IRS1, ITGA2,
JAG1, KDR, LFNG, LOX, LRP12, MED12, NOTCH1, NOTCH4, PAG1, PDGFB, PIM3, PLAT,
PMEPA1, PPP2CB, PRKCE, PRMT6, RAC3, RB1, RIPK3, RUNX1, S100A6, SERPINF1, SFRP2,
SHC2, SLC25A13, SLIT2, SNCA, SPARC, SPRY4, SRF, STK3, STK39, TBL1Y, THBS2, THBS4,
TIMP3, TMEM74B, TNFRSF11B, TNFSF14, TNXB, VEGFB
Table 3.3
ACKR1, ACTB, AHNAK, BATF, BOK, CCL14, CCL17, CD55, CMKLR1, COL1A1, COL1A2,
COL3A1, COL5A1, COL5A2, CRY1, DLL4, DNAJB14, EDIL3, ENG, FBN1, FGF13, FN1, HEY2,
HSPA9, IGFBP7, IRS1, ITGA2, JAG1, LFNG, LOX, LRP12, MED12, MMP2, MMS19, NOTCH4,
PAG1, PLAT, PMEPA1, PPP2CB, RAC3, RB1, RIPK3, RUNX1, S100A6, SFRP2, SHC2, SLIT2,
SPARC, SRF, THBS2, THBS4, TIMP2, TIMP3, TMEM74B, TNFRSF11B, TRIB1
Table 3.4
AHNAK, BATF, BOK, CCL14, CCL17, CD55, CMKLR1, COL1A1, COL1A2, COL3A1, COL5A1,
COL5A2, CRY1, DLL4, ENG, FGF13, HEY2, IGFBP7, IRS1, ITGA2, JAG1, LFNG, LRP12,
MED12, NOTCH4, PAG1, PLAT, PMEPA1, PPP2CB, RAC3, RB1, RIPK3, RUNX1, S100A6,
SHC2, SLIT2, SPARC, SRF, THBS2, THBS4, TIMP3, TMEM74B, TNFRSF11B
Table 3.5
ACTB, BATF, BOK, COL1A1, COL1A2, COL3A1, COL5A1, COL5A2, DNAJB14, FBN1, FGF13,
FN1, HEY2, HSPA9, IRS1, ITGA2, LOX, MED12, MMP2, MMS19, NOTCH4, PAG1, PLAT,
RAC3, RB1, RIPK3, RUNX1, SFRP2, SPARC, SRF, THBS4, TIMP3, TRIB1
Table 3.6
BATF, BOK, COL1A1, COL1A2, FGF13, HEY2, IRS1, ITGA2, MED12, NOTCH4, PAG1, PLAT,
RAC3, RB1, RIPK3, RUNX1, SPARC, SRF, THBS4, TIMP3
Table 3.7
ACTB, BATF, COL1A1, COL1A2, COL3A1, COL5A1, COL5A2, DNAJB14, FBN1, FGF13, FN1,
HSPA9, ITGA2, LOX, MMP2, MMS19, PAG1, PLAT, RAC3, RB1, RIPK3, RUNX1, SFRP2,
SPARC, SRF, THBS4, TIMP3, TRIB1
Table 3.8
BATF, COL1A1, FGF13, ITGA2, PAG1, PLAT, RAC3, RB1, RIPK3, RUNX1, SPARC, SRF,
THBS4, TIMP3
Table 3.9
ACTB, BATF, COL1A1, COL1A2, COL3A1, COL5A1, COL5A2, DNAJB14, FBN1, FN1, HSPA9,
ITGA2, LOX, MMP2, MMS19, PAG1, PLAT, RB1, RUNX1, SFRP2, SPARC, SRF, THBS4,
TIMP3, TRIB1
Table 3.10
BATF, COL1A1, ITGA2, PAG1, PLAT, RB1, RUNX1, SPARC, SRF, THBS4, TIMP3
Table 3.11
ACTB, BATF, DNAJB14, HSPA9, MMS19, PAG1, PLAT, RUNX1, SRF, THBS4, TRIB1
Table 3.12
BATF, PAG1, PLAT, RUNX1, SRF, THBS4

TABLES 4.1 TO 4.12
Table 4.1
ACSL4, ACTR3B, ADRA1D, AGT, AK3, AKT2, ALDOC, BCL2A1, CA9, CCDC103, CCL25,
CCL3, CCL5, CD47, CEBPB, CHGA, CHI3L1, DDX58, DHX58, EAF2, ER_013, ER_028,
ER_109, ER_154, ERBB2, FGF8, GATA1, GBP1, GJA1, GNLY, GRIN2A, GZMB, HAND1,
HDAC8, HLA_A, HLA_B, HLA_E, HNF1B, HSPA1L, ID2, IDH1, IFT52, IL2RB, IL6R, IRF2,
ISG15, ITPKB, JAK2, LAG3, LRIG1, MADD, MAX, MLLT3, MX1, MYBL1, NFE2L2, NFKB1,
NTRK1, ORM2, PFKFB3, PLA2G4A, PPID, PRF1, PSIP1, PTP4A1, PTPN5, QSOX2, RARB,
SLC11A1, SLC16A1, SLC3A1, SOCS4, SPOP, STAT1, TAP1, TAP2, TERF1, TLR3, TNFAIP3,
TNFRSF10C, TOP3A, UBB, VCAN, WNT7A, WWOX
Table 4.2
ACSL4, ACTR3B, AGT, AK3, AKT2, ALDOC, BCL2A1, CA9, CCDC103, CCL25, CD47,
CEBPB, CHI3L1, DDX58, DHX58, EAF2, ER_154, ERBB2, GJA1, GNLY, GRIN2A, HDAC8,
HLA_A, HLA_B, HSPA1L, ID2, IDH1, IL6R, IRF2, ITPKB, JAK2, LAG3, LRIG1, MADD, MAX,
MLLT3, MYBL1, NFKB1, NTRK1, ORM2, PFKFB3, PLA2G4A, PPID, PSIP1, PTP4A1, QSOX2,
RARB, SLC11A1, SLC16A1, SOCS4, SPOP, TAP1, TAP2, TERF1, TLR3, TNFAIP3, TOP3A,
UBB, VCAN, WWOX
Table 4.3
ACSL4, AGT, AK3, ALDOC, CA9, CCL5, CHI3L1, DHX58, ER_013, ER_028, ER_109, ER_154,
GNLY, GRIN2A, GZMB, HLA_A, HLA_B, HLA_E, HSPA1L, IDH1, IL2RB, IL6R, IRF2, ITPKB,
LRIG1, MADD, MAX, MYBL1, NFKB1, ORM2, PPID, PRF1, PSIP1, PTP4A1, QSOX2, RARB,
SPOP, TERF1, TLR3, TNFRSF10C, TOP3A, VCAN
Table 4.4
ACSL4, AGT, AK3, ALDOC, CA9, CHI3L1, DHX58, ER_154, GNLY, GRIN2A, HLA_A, HLA_B,
HSPA1L, IDH1, IL6R, IRF2, ITPKB, LRIG1, MADD, MAX, MYBL1, NFKB1, ORM2, PPID,
PSIP1, PTP4A1, QSOX2, RARB, SPOP, TERF1, TLR3, TOP3A, VCAN
Table 4.5
AGT, AK3, ALDOC, CCL5, CHI3L1, DHX58, ER_013, ER_028, ER_109, ER_154, GNLY,
GZMB, HLA_A, HLA_B, HLA_E, IDH1, IL2RB, IL6R, IRF2, LRIG1, MADD, NFKB1, ORM2,
PRF1, PSIP1, QSOX2, SPOP, TLR3, VCAN
Table 4.6
AGT, AK3, ALDOC, CHI3L1, DHX58, ER_154, GNLY, HLA_A, HLA_B, IDH1, IL6R, IRF2,
LRIG1, MADD, NFKB1, ORM2, PSIP1, QSOX2, SPOP, TLR3, VCAN
Table 4.7
AK3, CHI3L1, DHX58, ER_013, ER_028, ER_109, ER_154, HLA_A, HLA_B, HLA_E, IL6R,
IRF2, LRIG1, ORM2, PSIP1, QSOX2, SPOP
Table 4.8
AK3, CHI3L1, DHX58, ER_154, HLA_A, HLA_B, IL6R, IRF2, LRIG1, ORM2, PSIP1, QSOX2,
SPOP
Table 4.9
AK3, DHX58, HLA_A, HLA_B, HLA_E, IL6R, IRF2, LRIG1, PSIP1, QSOX2, SPOP
Table 4.10
AK3, DHX58, HLA_A, HLA_B, IL6R, IRF2, LRIG1, PSIP1, QSOX2, SPOP
Table 4.11
HLA_A, HLA_B, IL6R, IRF2, LRIG1, QSOX2, SPOP
Table 4.12
HLA_A, IL6R, IRF2, LRIG1, QSOX2, SPOP

TABLES 5.1 TO 5.12
Table 5.1
ACTB, ADAMTS1, ADIPOR1, ALKBH3, ATP5F1, BID, CAD, CCL17, CCL28, CCT4, COL1A1,
COL1A2, COL3A1, COL5A1, COL5A2, CRLF2, CXCL8, DIABLO, DNAJB14, EIF6, EOMES,
FASN, FBN1, FGFR3, FN1, GPAT2, GSN, HEY2, HRK, HSPA9, KDR, KRT7, LCN2, LOX,
MED12, MMP14, MMP2, MMS19, NKD1, NLRP3, NOD2, NSD1, NUMBL, P4HB, PIK3CA,
PMS1, PRKAA2, PTPN11, RAD51C, RUNX1, SELE, SERPINF1, SFRP2, SLC16A2, SLC45A3,
SPARC, SPRY2, STK3, TADA3, THBS4, TIE1, TIMP3, TK1, TMEM74B, TNFRSF8, TNXB,
TOP1, TRIB1, TSPAN13, XRCC5, YY1
Table 5.2
ADAMTS1, ADIPOR1, ALKBH3, ATP5F1, BID, CAD, CCL17, CCL28, CCT4, COL1A1,
COL1A2, COL3A1, COL5A1, CXCL8, DIABLO, EIF6, FASN, FGFR3, GPAT2, GSN, HEY2,
HRK, KDR, KRT7, LCN2, MED12, MMP14, NKD1, NLRP3, NOD2, NSD1, NUMBL, P4HB,
PIK3CA, PRKAA2, PTPN11, RAD51C, RUNX1, SELE, SERPINF1, SFRP2, SLC16A2, SLC45A3,
SPRY2, STK3, TADA3, THBS4, TIMP3, TK1, TMEM74B, TNFRSF8, TNXB, TOP1, TSPAN13,
XRCC5
Table 5.3
ACTB, ADAMTS1, ATP5F1, BID, CCL17, CCL28, COL1A1, COL1A2, COL3A1, COL5A1,
COL5A2, DNAJB14, EIF6, FBN1, FN1, GSN, HEY2, HRK, HSPA9, KDR, LCN2, LOX, MED12,
MMP14, MMP2, MMS19, NKD1, NOD2, PIK3CA, PRKAA2, PTPN11, RAD51C, RUNX1, SELE,
SERPINF1, SFRP2, SLC16A2, SPARC, THBS4, TIE1, TMEM74B, TNXB, TRIB1, YY1
Table 5.4
ADAMTS1, ATP5F1, BID, CCL17, CCL28, COL1A1, COL1A2, COL5A1, EIF6, GSN, HEY2,
HRK, KDR, LCN2, MED12, MMP14, NKD1, NOD2, PIK3CA, PRKAA2, PTPN11, RAD51C,
RUNX1, SELE, SERPINF1, SFRP2, SLC16A2, THBS4, TMEM74B, TNXB
Table 5.5
ACTB, ADAMTS1, ATP5F1, BID, CCL17, COL1A1, COL1A2, COL3A1, COL5A1, COL5A2,
DNAJB14, EIF6, FBN1, FN1, GSN, HEY2, HSPA9, LOX, MED12, MMP2, MMS19, PIK3CA,
PRKAA2, PTPN11, RAD51C, RUNX1, SERPINF1, SFRP2, SPARC, TMEM74B, TRIB1, YY1
Table 5.6
ADAMTS1, ATP5F1, BID, CCL17, COL1A1, COL1A2, EIF6, GSN, HEY2, MED12, PIK3CA,
PRKAA2, PTPN11, RAD51C, RUNX1, SERPINF1, TMEM74B
Table 5.7
ACTB, ADAMTS1, CCL17, COL1A1, COL1A2, COL3A1, COL5A1, COL5A2, DNAJB14, FBN1,
FN1, GSN, HSPA9, LOX, MED12, MMP2, MMS19, PIK3CA, PTPN11, RUNX1, SFRP2, SPARC,
TRIB1, YY1
Table 5.8
ADAMTS1, CCL17, COL1A1, GSN, MED12, PIK3CA, PTPN11, RUNX1
Table 5.9
ACTB, ADAMTS1, COL1A1, COL1A2, COL3A1, COL5A1, COL5A2, DNAJB14, FBN1, FN1,
HSPA9, LOX, MED12, MMP2, MMS19, PIK3CA, RUNX1, SFRP2, SPARC, TRIB1
Table 5.10
ADAMTS1, COL1A1, MED12, PIK3CA, RUNX1
Table 5.11
ACTB, ADAMTS1, DNAJB14, HSPA9, MED12, MMS19, PIK3CA, RUNX1, TRIB1
Table 5.12
ADAMTS1, MED12, PIK3CA, RUNX1

TABLES 6.1 TO 6.12
Table 6.1
ACSL4, AK3, AKT2, BCL2A1, CA9, CCL5, CD47, DDX58, DHX58, EAF2, GBP1, GNLY,
GZMB, HLA_A, HLA_B, HLA_E, IFT52, IL2RB, IL6R, IRF2, ISG15, JAK2, LAG3, MADD,
MLLT3, MX1, NFKB1, PRF1, PSIP1, SOCS4, STAT1, TAP1, TAP2, TERF1, TLR3
Table 6.2
ACSL4, AK3, AKT2, BCL2A1, CA9, CD47, DDX58, DHX58, EAF2, GNLY, HLA_A, HLA_B,
IL6R, IRF2, JAK2, LAG3, MADD, MLLT3, NFKB1, PSIP1, SOCS4, TAP1, TAP2, TERF1, TLR3
Table 6.3
ACSL4, AK3, CA9, CCL5, DHX58, GNLY, GZMB, HLA_A, HLA_B, HLA_E, IL2RB, IL6R,
IRF2, MADD, NFKB1, PRF1, PSIP1, TERF1, TLR3
Table 6.4
ACSL4, AK3, CA9, DHX58, GNLY, HLA_A, HLA_B, IL6R, IRF2, MADD, NFKB1, PSIP1,
TERF1, TLR3
Table 6.5
AK3, CCL5, DHX58, GNLY, GZMB, HLA_A, HLA_B, HLA_E, IL2RB, IL6R, IRF2, MADD,
NFKB1, PRF1, PSIP1, TLR3
Table 6.6
AK3, DHX58, GNLY, HLA_A, HLA_B, IL6R, IRF2, MADD, NFKB1, PSIP1, TLR3
Table 6.7
AK3, DHX58, HLA_A, HLA_B, HLA_E, IL6R, IRF2, PSIP1
Table 6.8
AK3, DHX58, HLA_A, HLA_B, IL6R, IRF2, PSIP1
Table 6.9
AK3, DHX58, HLA_A, HLA_B, HLA_E, IL6R, IRF2, PSIP1
Table 6.10
AK3, DHX58, HLA_A, HLA_B, IL6R, IRF2, PSIP1
Table 6.11
HLA_A, HLA_B, IL6R, IRF2
Table 6.12
HLA_A, IL6R, IRF2

TABLES 7
ER_013, ER_028

TABLES 8.1 TO 8.12
Table 8.1
ACTB, ATP5F1, BID, CCL17, COL1A1, COL1A2, COL3A1, COL5A1, COL5A2, CXCL8,
DNAJB14, FASN, FBN1, FN1, GSN, HEY2, HSPA9, KDR, LOX, MED12, MMP2, MMS19,
NUMBL, P4HB, RUNX1, SERPINF1, SFRP2, SPARC, STK3, THBS4, TIE1, TIMP3, TMEM74B,
TNXB, TOP1, TRIB1, YY1
Table 8.2
ATP5F1, BID, CCL17, COL1A1, COL1A2, COL3A1, COL5A1, CXCL8, FASN, GSN, HEY2,
KDR, MED12, NUMBL, P4HB, RUNX1, SERPINF1, SFRP2, STK3, THBS4, TIMP3, TMEM74B,
TNXB, TOP1
Table 8.3
ACTB, ATP5F1, BID, CCL17, COL1A1, COL1A2, COL3A1, COL5A1, COL5A2, DNAJB14,
FBN1, FN1, GSN, HEY2, HSPA9, KDR, LOX, MED12, MMP2, MMS19, RUNX1, SERPINF1,
SFRP2, SPARC, THBS4, TIE1, TMEM74B, TNXB, TRIB1, YY1
Table 8.4
ATP5F1, BID, CCL17, COL1A1, COL1A2, COL5A1, GSN, HEY2, KDR, MED12, RUNX1,
SERPINF1, SFRP2, THBS4, TMEM74B, TNXB
Table 8.5
ACTB, ATP5F1, BID, CCL17, COL1A1, COL1A2, COL3A1, COL5A1, COL5A2, DNAJB14,
FBN1, FN1, GSN, HEY2, HSPA9, LOX, MED12, MMP2, MMS19, RUNX1, SERPINF1, SFRP2,
SPARC, TMEM74B, TRIB1, YY1
Table 8.6
ATP5F1, BID, CCL17, COL1A1, COL1A2, GSN, HEY2, MED12, RUNX1, SERPINF1, TMEM74B
Table 8.7
ACTB, CCL17, COL1A1, COL1A2, COL3A1, COL5A1, COL5A2, DNAJB14, FBN1, FN1, GSN,
HSPA9, LOX, MED12, MMP2, MMS19, RUNX1, SFRP2, SPARC, TRIB1, YY1
Table 8.8
CCL17, COL1A1, GSN, MED12, RUNX1
Table 8.9
ACTB, COL1A1, COL1A2, COL3A1, COL5A1, COL5A2, DNAJB14, FBN1, FN1, HSPA9, LOX,
MED12, MMP2, MMS19, RUNX1, SFRP2, SPARC, TRIB1
Table 8.10
COL1A1, MED12, RUNX1
Table 8.11
ACTB, DNAJB14, HSPA9, MED12, MMS19, RUNX1, TRIB1
Table 8.12
MED12, RUNX1

The markers in Tables 2.1 to 2.12 are markers that are particularly indicative markers for a good prognosis in terms of pCR. The markers in Tables 3.1 to 3.12 are markers that are particularly indicative markers for a bad prognosis in terms of pCR. The markers in Tables 4.1 to 4.12 are markers that are particularly indicative markers for subjects benefiting from the cancer immunotherapy. The markers in Tables 5.1 to 5.12 are markers that are particularly indicative markers for subjects not benefiting from the cancer immunotherapy. The markers in Tables 6.1 to 6.12 are markers that are particularly indicative markers for a good prognosis in terms of pCR and for subjects benefiting from the cancer immunotherapy. The markers in Tables 7 are markers that are particularly indicative markers for a bad prognosis in terms of pCR and for subjects benefiting from the cancer immunotherapy. The markers in Tables 8.1 to 8.12 are markers that are particularly indicative markers for a bad prognosis in terms of pCR and for subjects not benefiting from the cancer immunotherapy. Hence, depending on desired prediction and/or prognosis, particular markers or marker combinations can in some embodiments be selected.

The neoplastic disease can be an early, non-metastatic neoplastic disease or a recurrent and/or metastatic neoplastic disease. As used herein, the term “recurrent” refers in particular to the occurrence of metastasis. Such metastasis may be distal metastasis that can appear after the initial diagnosis, even after many years, and therapy of a tumor, to local events such as infiltration of tumor cells into regional lymph nodes, or occurrence of tumor cells at the same site and organ of origin. The term “early” as used herein refers to non-metastatic diseases, in particular cancer. In one embodiment, the neoplastic disease is a non-metastatic disease.

In some embodiments, the neoplastic disease is cancer. For example, the cancer may include but is not limited to bladder cancer, breast cancer, cervical cancer, colon cancer, esophageal cancer, endometrial cancer, gastric cancer, glioblastoma, head and neck cancer, hepatocellular carcinoma, leukemia, lung cancer, lymphoma, melanoma, multiple myeloma, neuroblastoma, neuroendocrine cancer, ovarian cancer, pancreatic cancer, prostate cancer, rectal cancer, renal cell carcinoma, rhabdoid cancer, sarcomas, and urinary track cancer. In one embodiment, the neoplastic disease is a disease selected from the group consisting of breast cancer, lung cancer, renal cell carcinoma, melanoma, bladder cancer, urothelial carcinoma and Merkel-cell carcinoma. The method is in particular used in the context of breast cancer.

Hence, in a preferred embodiment, the neoplastic disease is breast cancer. Along with classification of histological type and grade, breast cancers are routinely evaluated for expression of hormone receptors (estrogen receptor (ER) and progesterone receptor (PR)) and for expression of HER2 (ErbB2). ER and PR are both nuclear receptors (they are predominantly located at cell nuclei, although they can also be found at the cell membrane). HER2, or human epidermal growth factor receptor type 2, is a receptor normally located on the cell surface.

In a more particular embodiment, the neoplastic disease is primary triple negative breast cancer (TNBC). As used herein, the term “triple negative” or “TN” refers to tumors (e.g., carcinomas), typically breast tumors, in which the tumor cells score negative (i.e., using conventional histopathology methods) for estrogen receptor (ER) and progesterone receptor (PR), both of which are nuclear receptors (i.e., they are predominantly located at cell nuclei), and the tumor cells are not amplified for epidermal growth factor receptor type 2 (HER2 or ErbB2), a receptor normally located on the cell surface. Furthermore, the term “triple negative breast cancer(s)” or “TN breast cancer(s)” encompasses carcinomas of differing histopathological phenotypes. For example, certain TN breast cancers are classified as “basal-like” (“BL”), in which the neoplastic cells express genes usually found in normal basal/myoepithelial cells of the breast, such as high molecular weight basal cytokeratins (CK, CK5/6, CK14, CK17), vimentin, p-cadherin, ccB crystallin, fascin and caveolins 1 and 2. Certain other TN breast cancers, however, have a different histopathological phenotype, examples of which include high grade invasive ductal carcinoma of no special type, metaplastic carcinomas, medullary carcinomas and salivary gland-like tumors of the breast.

As used herein, the terms “cancer immunotherapy” and “cancer immunotherapy treatment” are used interchangeably and refer to a treatment that uses the body [symbol] immune system, either directly or indirectly, to shrink or eradicate cancer. For example, the cancer immunotherapy may stimulate the immune system to treat cancer by improving on the system [symbol] natural ability to fight cancer by stimulating the body [symbol] own immune system by general means in order to boost the immune system to attack cancer cells. As another example, the cancer immunotherapy may exploit tumor antigens, i.e. the surface molecules of cancer cells such as proteins or other macromolecules and train the immune system to attack cancer cells by targeting the tumor antigens. The cancer immunotherapy as used herein may be selected from the group consisting of immune checkpoint inhibitors, chimeric antigen receptor (CAR)-T cell therapies and cancer vaccines. Monoclonal antibodies which are conventionally used in the treatment of cancer are particularly excluded from the cancer immunotherapy as provided herein. Thus, the cancer therapy as used in the context of the present invention does not include monoclonal antibodies that are traditionally and/or conventionally used in the treatment of cancer. The person skilled in the art knows traditional and/or conventional monoclonal antibodies that are used in cancer treatment. Such traditional and/or conventional monoclonal antibodies that are not encompassed by the cancer immunotherapy as provided herein include but are not limited to Bevacizumab (Avastin®), Cetuximab (Erbitux®), several naked antibodies such as Alemtuzumab (Campath®) and Trastuzumab (Herceptin®), several conjugated antibodies such as radiolabeled antibodies including ibritumomab tiutexan (Zevalin®), several chemolabeled antibodies including Brentuximab vedotin (Adcetris®), Ado-trastuzumab emtansine (Kadcyla®, also called TDM-1) and Denileukin diftitox (Ontak®) and several bispecific antibodies such as Blinatumomab (Blincyto).

In one embodiment, the cancer immunotherapy is, thus, selected from the group consisting of immune checkpoint inhibitor therapy, chimeric antigen receptor (CAR) T-Cell therapy and cancer vaccine therapy.

As used herein, the term “CAR T-cell therapy” or “chimeric antigen receptor T-cell therapy” refers to a type of treatment in which T-cells in a subject are changed ex vivo in such a manner so that they will attack cancer cells in vivo and/or trigger other parts of the immune system to destroy cancer cells. Such T-cells may be, for example, taken from blood of the subject and a gene for a special receptor that binds to a certain protein on the subject's cancer cell is added ex vivo. The special receptor may be a man-made receptor and is called a chimeric antigen receptor (CAR). The subject's own T-cells are used to make the CAR T-cells. The CAR T-cells may be grown ex vivo and returned to the subject, for example by infusion. The CAR T-cells may be able to identify specific cancer cell antigens. Since different cancer cells may have different antigens, each CAR may be made for a specific cancer antigen. For example, certain kinds of leukemia or lymphoma will have an antigen on the outside of the cancer cells called CD19. The CAR T-cell therapies to treat those cancers are made to connect to the CD-19 antigen and will not work for a cancer that does not have the CD19 antigen. Methods of producing CAR T-cells are well known in the art. For example, CAR T-cell therapies approved in the US include CAR T-cell therapies for advanced or recurrent acute lymphoblastic leukemia in children and young adults and for certain types of advanced or recurrent large B-cell lymphoma. In general, types of cancer in which CAR T-cell therapies are now being studied includes, for example, brain tumors (especially glioblastoma), breast cancer, acute myeloid leukemia, multiple myeloma, Hodgkin's lymphoma, neuroblastoma, CLL and pancreas cancer.

As used herein, the term “cancer vaccine” refers to a type of treatment in which the immune system's ability to recognize and destroy cancer antigens is boosted. Such cancer vaccines may comprise traditional vaccines that target the viruses that can cause certain cancers and may protect against these cancers, however they may not target the cancer cells directly. As such, strains of the human papilloma virus (HPV) have been linked to cervical, anal, throat, and some other cancers. Further, people who have chronic or long-term infections with the hepatitis B virus (HBV) may be at higher risk for liver cancer. Therefore, administration of a vaccine preventing HBV infection may also lower the risk of developing liver cancer. Moreover, cancer vaccines of the present invention may comprise vaccines for treating an existing cancer. For example, cancer vaccines may be produced by immunizing subjects against specific cancer antigens and thereby stimulate the immune system to attack and destroy the cancer cells. In a preferred embodiment of the present invention, the cancer vaccine is a cancer vaccine for treating an existing cancer. Examples of such cancer vaccines include but are not limited to Sipuleucel-T (Provenge) which is approved in the US and used to treat advanced prostate cancer. Several different types of cancer vaccines are investigated in clinical trials and studies including but not limited to tumor cell vaccines, antigen vaccines, dendritic cell vaccines, vector-based vaccines. Tumor cell vaccines may be made from actual cancer cells that have been removed from the subject during surgery. The cells may be modified (and killed) in the laboratory to increase the probability for them to become attacked by the immune system after they have been injected back into the subject. The subject's immune system may then attack these cells and any similar cells still in the body. Antigen vaccines may boost the immune system by using only one or a few antigen(s), rather than whole tumor cells. The antigens are for example proteins or peptides. Dendritic cell vaccines may be made from the person in whom they will be used and break down cancer cells into antigens that are presented by T cells which may start an immune reaction against any cells in the body that contain these antigens. Vector based vaccines may use special delivery systems (called vectors) to make them more effective. Such vectors may include but are not limited to viruses, bacteria, yeast cells, or other structures that can be used to effectively deliver antigens into the body. In general, types of cancer in which cancer vaccines are now being studied includes, for example, brain tumors (especially glioblastoma), breast cancer, cervical cancer, colorectal cancer, kidney cancer, lung cancer, lymphoma, melanoma, pancreas cancer and prostate cancer.

In one embodiment, the cancer immune therapy comprises treatment with an immune checkpoint inhibitor. As used herein, the term “immune checkpoint inhibitor” refers to a substance that blocks the activity of molecules involved in attenuating the immune response, i.e. so called immune checkpoint proteins. The term “immune checkpoint protein” is known in the art. Within the known meaning of this term it will be clear to the skilled person that on the level of “immune checkpoint proteins” the immune system provides inhibitory signals to its components in order to balance immune reactions. Known immune checkpoint proteins comprise CTLA-4, PD1 and its ligands PD-L1 and PD-L2 and in addition LAG-3, BTLA, B7H3, B7H4, TIM3, KIR. The pathways involving LAG3, BTLA, B7H3, B7H4, TIM3, and KIR are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Within the present invention, inhibition by an immune checkpoint inhibitor includes reduction of function and full blockade. Immune checkpoint proteins are described in the art (see for instance Pardoll, 2012. Nature Rev. cancer 12: 252-264). The designation immune checkpoint includes the experimental demonstration of stimulation of an antigen-receptor triggered T lymphocyte response by inhibition of the immune checkpoint protein in vitro or in vivo, e.g. mice deficient in expression of the immune checkpoint protein demonstrate enhanced antigen-specific T lymphocyte responses or signs of autoimmunity (such as disclosed in Waterhouse et al., 1995. Science 270:985-988; Nishimura et al., 1999. Immunity 11:141-151). It may also include demonstration of inhibition of antigen-receptor triggered CD4+ or CD8+ T cell responses due to deliberate stimulation of the immune checkpoint protein in vitro or in vivo (e.g. Zhu et al., 2005. Nature Immunol. 6:1245-1252). Preferred immune checkpoint protein inhibitors are antibodies that specifically recognize immune checkpoint proteins. Examples of immune checkpoint inhibitors include, but are not limited to inhibitors of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GALS, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA and VTCN1. As the skilled person will know, alternative and/or equivalent names may be in use for certain antibodies mentioned above. Such alternative and/or equivalent names are interchangeable in the context of the present invention.

In one embodiment, the immune checkpoint inhibitor is selected from the group consisting of a drug targeting CTLA4, a drug targeting PD-1 and a drug targeting PD-L1. For example ipilimumab is a fully human CTLA-4 blocking antibody presently marketed under the name Yervoy (Bristol-Myers Squibb). A second CTLA-4 inhibitor is tremelimumab (referenced in Ribas et al., 2013, J. Clin. Oncol. 31:616-22). Examples of PD-1 inhibitors include without limitation humanized antibodies blocking human PD-1 such as lambrolizumab (e.g. disclosed as hPD109A and its humanized derivatives h409A11, h409A16 and h409A17 in WO2008/156712; Hamid et al., N. Engl. J. Med. 369: 134-144 2013,), or pidilizumab (disclosed in Rosenblatt et al., 2011. J Immunother. 34:409-18), as well as fully human antibodies such as nivolumab (previously known as Opdivo or MDX-1106 or BMS-936558, Topalian et al., 2012. N. Eng. J. Med. 366:2443-2454, disclosed in U.S. Pat. No. 8,008,449 B2). Other PD-1 inhibitors may include presentations of soluble PD-1 ligand including without limitation PD-L2 Fc fusion protein also known as B7-DC-Ig or AMP-244 (disclosed in Mkrtichyan M, et al. J Immunol. 189:2338-47 2012), Pembrolizumab (also known as Keytruda), Cemiplimab (also known as Libtayo) and other PD-1 inhibitors presently under investigation and/or development for use in therapy. In addition, immune checkpoint inhibitors may include without limitation humanized or fully human antibodies blocking PD-L1 such as MEDI-4736 (disclosed in WO2011066389 A1), MPDL328 OA (disclosed in U.S. Pat. No. 8,217,149 B2) and MIH1 (Affymetrix obtainable via eBioscience (16.5983.82)), Atezolizumab (Tecentriq), Avelumab (Bavencio), Durvalumab (Imfinzi) and other PD-L1 inhibitors presently under investigation. As the skilled person will know, alternative and/or equivalent names may be in use for certain immune checkpoint inhibitors mentioned above. Such alternative and/or equivalent names are interchangeable in the context of the present invention.

In another embodiment, the immune checkpoint inhibitor is a therapeutic antibody. In the present invention the term “antibody” is used in the broadest sense and specifically covers, but is not limited to, monoclonal antibodies (including full length monoclonal antibodies), polyclonal antibodies, and multispecific antibodies (e.g., bispecific antibodies) and binding fragments thereof. In particular, monoclonal antibodies that are traditionally and/or conventionally used for the treatment of cancer but not in a cancer immunotherapy are particularly excluded in the context of the present invention. “Antibody fragment” and “antibody binding fragment” mean antigen-binding fragments of an antibody, typically including at least a portion of the antigen binding or variable regions (e.g. one or more CDRs) of the parental antibody. An antibody fragment retains at least some of the binding specificity of the parental antibody. Therefore, as is clear for the skilled person, “antibody fragments” in many applications may substitute antibodies and the term “antibody” should be understood as including “antibody fragments” when such a substitution is suitable. Examples of antibody fragments include, but are not limited to, Fab, Fab′, F(ab′)2, and Fv fragments; diabodies; linear antibodies; single-chain antibody molecules, e.g., sc-Fv, unibodies or duobodies (technology from Genmab); nanobodies (technology from Ablynx); domain antibodies (technology from Domantis); and multispecific antibodies formed from antibody fragments. Engineered antibody variants are reviewed in Holliger and Hudson, 2005, Nat. Biotechnol. 23:1126-1136. In a preferred embodiment, the immune checkpoint inhibitor is an anti-CTLA4 antibody, an anti-PD-1 antibody or an anti-PD-L1 antibody. In a more preferred embodiment, the immune checkpoint inhibitor is selected from the group consisting of ipilimumab, nivolumab, pembrolizumab, atezolizumab, avelumab, durvalumab, cemiplimab, lambrolizumab, pidilizumab or a combination thereof.

For the purposes of the present invention the “subject” (or “patient”) may be a mammal. In the context of the present invention, the term “subject” includes both humans and other mammals. Thus, the herein provided methods are applicable to both human and animal subjects, i.e. the method can be used for medical and veterinary purposes. Accordingly, said subject may be an animal such as a mouse, rat, hamster, rabbit, guinea pig, ferret, cat, dog, sheep, bovine species, horse, camel, or primate. Most preferably the subject is human. In one embodiment, the subject is a subject suffering from or being at risk of developing a neoplastic disease. In a preferred embodiment, the subject is suffering from or being at risk of developing a recurrent neoplastic disease. In another embodiment, the subject is suffering from or being at risk of developing a non-metastatic neoplastic disease, such as non-metastatic cancer. For example, the subject may be suffering from or being at risk of developing a neoplastic disease selected from the group consisting of breast cancer, lung cancer, renal cell carcinoma, melanoma, bladder cancer, urothelial carcinoma, Merkel-cell carcinoma and breast cancer. Preferably, the subject may be suffering from or being at risk of developing a neoplastic disease, wherein the neoplastic disease is breast cancer, for example triple negative breast cancer (TNBC).

As used herein, the terms “sample” or “biological sample” as are used interchangeably and refer to a sample obtained from the subject. The sample may be of any biological tissue or fluid suitable for carrying out the method of the present invention, i.e. for assessing whether a subject suffering from or being at risk of developing a neoplastic disease, in particular breast cancer, will respond or be resistant to and/or benefit from the cancer immunotherapy treatment and/or for assessing the outcome of said patient to the cancer immunotherapy treatment. However, typically, once the subject's is determined to have a response and/or benefit and/or good outcome with the cancer immunotherapy treatment according to the methods of the present invention, the subject will receive the cancer immunotherapy treatment as soon as possible.

In particular, the sample may be obtained from any tissue and/or fluid of a subject suffering from or being at risk of developing a neoplastic disease. Preferably, the tissue and/or fluid of the sample may be taken from any material of the neoplastic disease and/or from any material associated with the neoplastic disease. Such a sample may, for example, comprise cells obtained from the subject. In one embodiment, the sample may be a tumor sample. A “tumor sample” is a biological sample containing tumor cells, whether intact or degraded. In one embodiment, the sample is a tumor sample obtained from said subject. The sample may also be a bodily fluid. Such fluids may include the lymph. In one embodiment, the sample is a lymph node sample obtained from said subject. In another embodiment, the sample is a tumor sample or a lymph node sample obtained from said subject.

The sample may also include sections of tissues. Such sections of tissues also encompass frozen or fixed sections. These frozen or fixed sections may be used, e.g. for histological purposes. In one embodiment, the sample from said subject is a formalin-fixed paraffin embedded sample or a fresh-frozen sample.

A sample to be analyzed may be taken by aspiration or punctuation, excision or by any other surgical method leading to biopsy or resected cellular material. In one embodiment, in the sample obtained from said subject the expression levels of at least two, at least three, at least four, at least five, at least ten, at least twenty markers related to immune response and/or a marker related to antigen-presentation of a tumor cell, are determined.

For example, a combination of at least two, at least three, at least four, at least five, at least ten, at least twenty markers related to immune response and/or a marker related to antigen-presentation of a tumor cell may be determined, wherein said at least two, at least three, at least four, at least five, at least ten, at least twenty markers may comprise an at least one marker selected from List A of any of Tables 9.1 to 9.34 and an at least second marker selected from List B of the same Table of any of Tables 9.1 to 9.34 as the at least one marker.

TABLES 9.1 TO 9.34

| | List A | List B |
|---|---|---|
| 9.1 | MELK, PSIP1 | SOCS4 |
| 9.2 | APOL3, CCL5, CXCL10, ETV7, GBP1, | BATF, CASP10, CCR5, CD2, CD27, |
| | HLA_A, HLA_B, STAT1, TAP1, TAP2, | GZMB, IL2RB, IRF1, IRF4, PRF1 |
| | TYMP | |
| 9.3 | APOL3, CD74, CTSS, CXCL10, CYBB, | RB1 |
| | GBP1, HLA_A, HLA_B, HLA_E, STAT1, | |
| | TAP1 | |
| 9.4 | APOL3, CCL5, CD74, CXCL10, CXCL9, | COMP, F2R, IGF1, SFRP2, SFRP4, THBS4, |
| | GBP1, HLA_A, HLA_B, HLA_E, STAT1, | ZEB1 |
| | TAP1 | |
| 9.5 | CD74, CTSS, GBP1, HLA_A, HLA_B, | TBL1X |
| | HLA_E, STAT1, TAP1 | |
| 9.6 | APOL3, CCL5, CXCL10, ETV7, GBP1, | COL1A2, COL5A1, COL5A2, PDGFRB, |
| | HLA_A, HLA_B, STAT1, TAP1, TAP2, | PLAT, THY1, TIMP2 |
| | TYMP | |
| 9.7 | CCR5, CD27, CD38, CD79A, IL10RA, | CD27, CD3D, CMKLR1, FLT3LG, IRF4, |
| | IL2RB, IL2RG, IRF1, IRF4, PIM2, SLAMF7 | RIPK3, TNFRSF1B |
| 9.8 | COMP, F2R, IGF1, SFRP2, SFRP4, THBS4, | CCR2, CTLA4, IL6R, MAP4K1, TBX21, |
| | ZEB1 | TNFRSF17 |
| 9.9 | CCL5, CXCL10, ETV7, IRF1, LAG3, STAT1, | TBL1X |
| | TAP1 | |
| 9.10 | APOL3, IFIT2, IRF7, LAG3, MX1, OAS1, | TIFA |
| | OASL | |
| 9.11 | APOL3, CD74, CTSS, CXCL10, CYBB, | COMP, F2R, IGF1, SFRP2, SFRP4, THBS4, |
| | GBP1, HLA_A, HLA_B, HLA_E, STAT1, | ZEB1 |
| | TAP1 | |
| 9.12 | APOL3, CCL5, CD74, CTSS, CXCL10, | ADM, ANGPTL4, BNIP3, CA9 |
| | CXCL9, FGL2, GBP1, HLA_A, STAT1, | |
| | TAP1 | |
| 9.13 | ADAMTS1 | PIK3CA |
| 9.14 | ACTB, DNAJB14, DNAJC7, HSPA9, | BID |
| | LAMA5, MMS19, RUNX1, TICAM1, TRIB1, | |
| | WASL, YY1 | |
| 9.15 | HEY2 | CHI3L1 |
| 9.16 | CASP1, CD274, IRF1, IRF2, PIK3R5, | AQP9, IL1B, NLRP3, NOD2, SNAI3, |
| | TBX21, TLR3 | TLR2, TNFRSF9 |
| 9.17 | ATP7B, DHH, GATA4, JPH3, TIE1, | CASP1, GBP7, GNGT2, IFNG, IRF1, IRF2, |
| | TMEM74B, TNNI3 | TLR3 |
| 9.18 | ACTB, DNAJB14, DNAJC7, HSPA9, | SPOP |
| | LAMA5, MMS19, RUNX1, TICAM1, TRIB1, | |
| | WASL, YY1 | |
| 9.19 | CCR2, CTLA4, IL6R, MAP4K1, TBX21, | CCL17, ESR2, IL12B, LTA, MADCAM1, |
| | TNFRSF17 | MFNG, MS4A1, NR0B2, SERPINA9, |
| | | SNAI3, XCR1 |
| 9.20 | CASP1, CD86, DHX58, IFIT2, IRF7, OAS1, | COL1A1, COL1A2, FBN1, MMP2, |
| | OASL | SERPINF1, SFRP2, SFRP4 |
| 9.21 | COL1A1, COL1A2, COL3A1, COL5A1, | ATP5F1 |
| | COL5A2, FBN1, FN1, LOX, MMP2, SFRP2, | |
| | SPARC | |
| 9.22 | ADAMTS1 | ITPKB |
| 9.23 | ADAMTS1 | PIK3CA |
| 9.24 | MED12 | ACTB, ANAPC2, APPBP2, ARAF, |
| | | ATXN1, DNAJC7, GSN, MAP7D1, |
| | | MMS19, MT2A, YY1 |
| 9.25 | HEY2 | RAD51C |
| 9.26 | CASP1, CD274, IRF1, IRF2, PIK3R5, | CCL17, ESR2, IL12B, LTA, MADCAM1, |
| | TBX21, TLR3 | MFNG, MS4A1, NR0B2, SERPINA9, |
| | | SNAI3, XCR1 |
| 9.27 | ACTB, DNAJB14, DNAJC7, HSPA9, | BID |
| | LAMA5, MMS19, RUNX1, TICAM1, TRIB1, | |
| | WASL, YY1 | |
| 9.28 | ATF4, PTPN11, SOX2, TDG, TXNRD1 | ACTB, ANAPC2, APPBP2, ARAF, |
| | | ATXN1, DNAJC7, GSN, MAP7D1, |
| | | MMS19, MT2A, YY1 |
| 9.29 | HEY2 | EIF6 |
| 9.30 | CD74, CTSS, GBP1, HLA_A, HLA_B, | MED12 |
| | HLA_E, STAT1, TAP1 | |
| 9.31 | APOL3, CD74, CTSS, CXCL10, CYBB, | LRIG1 |
| | GBP1, HLA_A, HLA_B, HLA_E, STAT1, | |
| | TAP1 | |
| 9.32 | HEY2 | MED12 |
| 9.33 | CD74, CTSS, GBP1, HLA_A, HLA_B, | LRIG1 |
| | HLA_E, STAT1, TAP1 | |
| 9.34 | APOL3, CD74, CTSS, CXCL10, CYBB, | CHI3L1 |
| | GBP1, HLA_A, HLA_B, HLA_E, STAT1, | |
| | TAP1 | |

In one embodiment, the sample is an estrogen receptor (ER) negative and/or a HER2 negative sample. As outlined in detail above, ER is a nuclear receptor (predominantly located at cell nuclei, although it can also be found at the cell membrane). HER2, or human epidermal growth factor receptor type 2, is a receptor normally located on the cell surface. In particular breast cancers are associated with a reduced or lack of expression of hormone receptors (estrogen receptor (ER)) and/or for expression of HER2 (ErbB2). Thus, a sample that is an estrogen receptor negative and/or a HER2 negative sample may be a sample obtained from a subject suffering from or being at risk of developing breast cancer. For example, the subject may suffer from or being at risk at developing TNBC.

As used herein, the term “expression level of the at least one marker” refers to the quantity of the molecular entity of the marker in a sample that is obtained from the subject. In other words, the concentration of the marker is determined in the sample. It is also envisaged that a fragment of the marker can be detected and quantified. Thus, it is apparent to the person skilled in the art, in order to determine the expression of a marker, parts and fragments of said marker can be used instead. Suitable method to determine the expression level of the at least one marker are described herein below in detail. As used herein, the term “marker” relates to measurable and quantifiable biological markers which serve as indices for health- and physiology-related assessments, such as a disease/disorder/clinical condition risk. Furthermore, a marker is defined as a characteristic that is objectively measured and evaluated as an indicator of normal biological processes, pathogenic processes, or pharmacologic responses to a therapeutic intervention. As discussed herein above a biomarker may be measured on a biological sample (e.g., as a tissue test).

In one embodiment, the expression level of the at least one marker is the protein expression level or the RNA expression level, preferably mRNA expression level. For example, the expression level refers to a determined level of gene expression. A “gene” is a set of segments of nucleic acid that contains the information necessary to produce a functional RNA product. A “gene product” is a biological molecule produced through transcription or expression of a gene, e.g., an mRNA, cDNA or the translated protein. An “mRNA” is the transcribed product of a gene and shall have the ordinary meaning understood by a person skilled in the art. A “molecule derived from an mRNA” is a molecule which is chemically or enzymatically obtained from an mRNA template, such as cDNA. The expression level may be a determined level of protein, RNA, or mRNA expression as an absolute value or compared to a reference gene, to the average of two or more reference value, or to a computed average expression value or to another informative protein, RNA or mRNA without the use of a reference sample.

The gene names as used in the context of the present invention refer to gene names according to the official gene symbols provided by the HGNC (HUGO Gene Nomenclature Committee) and as used by the NCBI (National Center for Biotechnology Information) with the exception of the markers with the official gene names “HLA-A”, “HLA-B” and “HLA-E” which are herein designated “HLA_A”, “HLA_B” and “HLA_E”, respectively. The marker as identified in Table 1, Table 2.1 to Table 2.12, Table 3.1 to Table 3.12, Table 4.1 to Table 4.12, Table 5.1 to Table 5.12, Table 6.1 to Table 6.12, Table 7, Table 8.1 to Table 8.12, Table 9.1 to Table 9.34 and Table 10.1 and Table 10.2 refer to gene names. When referring to markers of the present invention as identified by the gene names in the above Tables, the person skilled in the art how to derive the respective RNA, in particular the mRNA, or the protein of the marker identified by its gene name. For example, the skilled person knows from the gene name RUNX2 how to identify the corresponding RNA, in particular the mRNA, or the protein transcribed or translated by the gene RUNX2.

In one embodiment, the expression level is the RNA expression level, preferably mRNA expression level, and is determined by at least one of a hybridization based method, a PCR based method, a microarray based method, a sequencing and/or next generation sequencing approach. The term “a PCR based method” as used herein refers to methods comprising a polymerase chain reaction (PCR). This is a method of exponentially amplifying nucleic acids, e.g. DNA by enzymatic replication in vitro. As PCR is an in vitro technique, it can be performed without restrictions on the form of DNA, and it can be extensively modified to perform a wide array of genetic manipulations. When it comes to the determination of expression levels, a PCR based method may for example be used to detect the presence of a given mRNA by (1) reverse transcription of the complete mRNA pool (the so called transcriptome) into cDNA with help of a reverse transcriptase enzyme, and (2) detecting the presence of a given cDNA with help of respective primers. This approach is commonly known as reverse transcriptase PCR (rtPCR). Moreover, PCR-based methods comprise e.g. real time PCR, and, particularly suited for the analysis of expression levels, kinetic or quantitative PCR (qPCR).

The term “Quantitative PCR” (qPCR)” refers to any type of a PCR method which allows the quantification of the template in a sample. Quantitative real-time PCR comprise different techniques of performance or product detection as for example the TaqMan technique or the LightCycler technique. The TaqMan technique, for examples, uses a dual-labelled fluorogenic probe. The TaqMan real-time PCR measures accumulation of a product via the fluorophore during the exponential stages of the PCR, rather than at the end point as in conventional PCR. The exponential increase of the product is used to determine the threshold cycle, CT, e.g., the number of PCR cycles at which a significant exponential increase in fluorescence is detected, and which is directly correlated with the number of copies of DNA template present in the reaction. The set up of the reaction is very similar to a conventional PCR, but is carried out in a real-time thermal cycler that allows measurement of fluorescent molecules in the PCR tubes. Different from regular PCR, in TaqMan real-time PCR a probe is added to the reaction, e.g., a single-stranded oligonucleotide complementary to a segment of 20-60 nucleotides within the DNA template and located between the two primers. A fluorescent reporter or fluorophore (e.g., 6-carboxyfluorescein, acronym: FAM, or tetrachlorofluorescin, acronym: TET) and quencher (e.g., tetramethylrhodamine, acronym: TAMRA, of dihydrocyclopyrroloindole tripeptide ‘black hole quencher’, acronym: BHQ) are covalently attached to the 5′ and 3′ ends of the probe, respectively. The close proximity between fluorophore and quencher attached to the probe inhibits fluorescence from the fluorophore. During PCR, as DNA synthesis commences, the 5′ to 3′ exonuclease activity of the Taq polymerase degrades that proportion of the probe that has annealed to the template. Degradation of the probe releases the fluorophore from it and breaks the close proximity to the quencher, thus relieving the quenching effect and allowing fluorescence of the fluorophore. Hence, fluorescence detected in the real time PCR thermal cycler is directly proportional to the fluorophore released and the amount of DNA template present in the PCR.

As used herein, the term “hybridization based method” refers to a method, wherein complementary, single-stranded nucleic acids or nucleotide analogues may be combined into a single double stranded molecule. Nucleotides or nucleotide analogues will bind to their complement under normal conditions, so two complementary strands will bind to each other. In bioanalytics, very often labeled, single stranded probes are in order to find complementary target sequences. If such sequences exist in the sample, the probes will hybridize to said sequences which can then be detected due to the label. Other hybridization based methods comprise microarray and/or biochip methods. For example, probes may be immobilized on a solid phase, which is then exposed to a sample. If complementary nucleic acids exist in the sample, these will hybridize to the probes and can thus be detected. These approaches are also known as “array based methods”. Yet another hybridization based method is PCR, which is described above. When it comes to the determination of expression levels, hybridization based methods may for example be used to determine the amount of mRNA for a given gene. An oligonucleotide capable of specifically binding sequences a gene or fragments thereof relates to an oligonucleotide which specifically hybridizes to a gene or gene product, such as the gene's mRNA or cDNA or to a fragment thereof. To specifically detect the gene or gene product, it is not necessary to detect the entire gene sequence. A fragment of about 20-150 bases will contain enough sequence specific information to allow specific hybridization.

By “array” or “matrix” an arrangement of addressable locations or “addresses” on a device is meant. The locations can be arranged in two dimensional arrays, three dimensional arrays, or other matrix formats. The number of locations can range from several to at least hundreds of thousands. Most importantly, each location represents a totally independent reaction site. Arrays include but are not limited to nucleic acid arrays, protein arrays and antibody arrays. A “nucleic acid array” refers to an array containing nucleic acid probes, such as oligonucleotides, nucleotide analogues, polynucleotides, polymers of nucleotide analogues, morpholino oligomers or larger portions of genes. The nucleic acid and/or analogue on the array is preferably single stranded. Arrays wherein the probes are oligonucleotides are referred to as “oligonucleotide arrays” or “oligonucleotide chips.” A “microarray,” herein also refers to a “biochip” or “biological chip”, an array of regions having a density of discrete regions of at least about 100/cm², and preferably at least about 1000/cm².

In one embodiment, the expression level of the at least one marker may be the protein level. It is clear to the person skilled in the art that a reference to a nucleotide sequence may comprise reference to the associated protein sequence which is coded by said nucleotide sequence. The expression level of a protein may be measured indirectly, e.g. by obtaining a signal wherein the signal strength is correlated to the amount of mRNA transcripts of that gene or it may be obtained directly at a protein level, e.g., by immunohistochemistry, CISH, ELISA (enzyme linked immunoassay), RIA (radioimmunoassay) or the use of protein microarrays, two-hybrid screening, blotting methods including western blot, one- and two dimensional gel electrophoresis, isoelectric focusing as well as methods being based on mass spectrometry like MALDI-TOF and the like. The term “immunohistochemistry” or IHC refers to the process of localizing proteins in cells of a tissue section exploiting the principle of antibodies binding specifically to antigens in biological tissues. Immunohistochemical staining is widely used in the diagnosis and treatment of cancer. Specific molecular markers are characteristic of particular cancer types. IHC is also widely used in basic research to understand the distribution and localization of biomarkers in different parts of a tissue.

Quantitative methods such as targeted RNA sequencing, modified nuclease protection assays, hybridization-based assays and quantitative PCR are particularly preferred herein.

In one embodiment, the prediction of the response, resistance, benefit and/or outcome is for a combination of the immune checkpoint inhibitor treatment with a non-chemotherapy and/or a chemotherapy, preferably a neoadjuvant chemotherapy. As used herein, the term “chemotherapy” refers to various treatment modalities affecting cell proliferation and/or survival. The treatment may include administration of alkylating agents, antimetabolites, anthracyclines, plant alkaloids, topoisomerase inhibitors, and other antitumor agents, including monoclonal antibodies and kinase inhibitors. As used herein, the term “neoadjuvant chemotherapy” relates to a systemic preoperative therapy regimen consisting of a panel of hormonal, chemotherapeutic and/or antibody agents, which is aimed to shrink the primary tumor, thereby rendering local therapy (surgery or radiotherapy) less destructive or more effective, enabling breast conserving surgery and evaluation of responsiveness of tumor sensitivity towards specific agents in vivo, and which is also aimed to eradicate micrometastasis (tumor cells spread throughout the body), thereby preventing from recurrence and improving survival. The present invention also includes a chemotherapy, wherein the chemotherapy is a monotherapy, i.e. comprising one or more chemotherapeutic agents but not a surgical intervention. In this case, the subject may be a subject, wherein the neoplastic disease is a metastatic cancer disease.

As used herein, the term “non-chemotherapy” refers to a type of therapy to treat cancer which does not comprise a chemotherapeutic agent. For example, non-chemotherapies may include but are not limited to surgery, hormone therapy, radiation, targeted therapy, poly ADP ribose polymerase (PARP) inhibitors, cyclin dependent kinase (CDK) inhibitors, such as CDK4/6 inhibitors and combinations thereof. The person skilled in the art knows which non-chemotherapeutic agents can be applied in a non-chemotherapy to treat subjects suffering from cancer.

In one embodiment, the method of the invention further comprises the prediction of the response or resistance to and/or benefit from a cancer immunotherapy treatment in a therapeutic regimen. As used herein, the term “regimen” and “therapy regimen” may be used interchangeably and refer to a timely sequential or simultaneous administration of compounds and/or surgical interventions. The composition of a therapy regimen may further comprise constant or varying dose of one or more compounds, a particular timeframe of application and frequency of administration within a defined therapy window. Such compounds may comprise compounds applied in non-chemotherapy and/or chemotherapy and include but are not limited to anti-tumor, and/or anti vascular, and/or immune stimulating, and/or blood cell proliferative agents, and/or radiation therapy, and/or hyperthermia, and/or hypothermia for cancer therapy. The administration of these can be performed in an adjuvant and/or neoadjuvant mode. Currently various combinations of various drugs and/or physical methods, and various schedules are under investigation. The term “adjuvant” relates to a postoperative systemic therapy regimen consisting of a panel of hormonal, chemotherapeutic and/or antibody agents, which is aimed to eradicate micrometastasis (tumor cells spread throughout the body), thereby preventing from recurrence and improving survival. In one embodiment, the therapy regimen is for cancer therapy. The administration of the therapy regimen may be performed in an adjuvant and/or neoadjuvant mode. In a preferred embodiment, the therapy regiment may be performed in a neoadjuvant mode. In one embodiment, the non-chemotherapy and/or chemotherapy is concomitant with and/or sequential to the checkpoint inhibitor treatment. For example, the therapeutic regimen comprises the administration of a non-chemotherapy and/or a chemotherapy and cancer immunotherapy, wherein the non-chemotherapy and/or the chemotherapy, including neoadjuvant therapy, is administered weekly or every two weeks for at least 12 weeks, preferably for at least 20 weeks and wherein the cancer immunotherapy treatment is given preferably every four weeks when starting the chemotherapy, wherein immune checkpoint therapy is started:
a) when starting the non-chemotherapy and/or the chemotherapy, including neoadjuvant therapy, or
b) prior to the start of the non-chemotherapy and/or the chemotherapy, including neoadjuvant therapy, preferably 3 to 28 days prior to the start of the non-chemotherapy and/or chemotherapy, including neoadjuvant therapy, more preferably 7 to 21 days prior to the start of the non-chemotherapy and/or the chemotherapy, most preferably 14 days prior to the start of the non-chemotherapy and/or the chemotherapy.

In one embodiment, the method is a method for therapy monitoring. As used herein, the term “therapy monitoring” in the context of the present invention refers to the monitoring and/or adjustment of a therapeutic treatment (here: particularly the treatment with a cancer immunotherapy) of said patient. “Monitoring” relates to keeping track of an already diagnosed disease, disorder, complication or risk, e.g. to analyze the progression of the disease or the influence of a particular treatment on the progression of disease or disorder. In the present invention, the terms “risk assessment” and “risk stratification” relate to the grouping of subjects into different risk groups according to their further prognosis. Risk assessment also relates to stratification for applying preventive and/or therapeutic measures.

In one embodiment, the response, benefit and/or outcome to be predicted or prognosticated is at least 12 weeks, at least 14 weeks, at least 20 weeks, at least 22 weeks after the start of the cancer immunotherapy treatment, more preferably after surgery. As used in the context of the present invention, the response, resistance benefit and/or outcome to be predicted or prognosticated refers to the response or resistance to, benefit from and/or outcome of the treatment with the cancer immunotherapy. In one embodiment, the the response, resistance, benefit and/or outcome to be predicted refers to the response or resistance to, benefit from and/or outcome of the treatment with the cancer immunotherapy with a non-chemotherapy and/or a chemotherapy, preferably a neoadjuvant therapy.

As used herein, the term “response” refers to any response to the treatment with the cancer immunotherapy. Non-limiting examples commonly used in oncology to evaluate the response of the subject to a therapy may be a change in tumor mass and/or volume and/or prolongation of time to distant metastasis or time to death following treatment. As used herein, “benefit” from a given therapy is an improvement in health or wellbeing that can be observed in patients under said therapy, but it is not observed in patients not receiving this therapy. Non-limiting examples commonly used in oncology to gauge a benefit from therapy are survival, disease free survival, metastasis free survival, disappearance of metastasis, tumor regression, and tumor remission. Vice versa, the term “resistance” as used herein refers to any non-response and or non-benefit to the treatment with the cancer immunotherapy. Non-limiting examples commonly used in oncology to evaluate the resistance of the subject to a therapy may be a change in tumor mass and/or volume and/or shorter time to distant metastasis or time to death following treatment.

The benefit and/or response or resistance may be assessed in a neoadjuvant situation where the size of a tumor after systemic intervention can be compared to the initial size and dimensions as measured by CT, PET, mammogram, ultrasound or palpation, usually recorded as “clinical response” of a patient. Response or resistance and/or benefit may also be assessed by caliper measurement or pathological examination of the tumor after biopsy or surgical resection. Response or resistance and/or benefit may be recorded in a quantitative fashion like percentage change in tumor volume or in a qualitative fashion like “no change” (NC), “partial remission” (PR), “complete remission” (CR) or other qualitative criteria. Assessment of tumor response or resistance and/or benefit may be done early after the onset of neoadjuvant therapy e.g. after a few hours, days, weeks or preferably after a few months. A typical endpoint for response or resistance and/or benefit assessment is upon termination of neoadjuvant chemotherapy or upon surgical removal of residual tumor cells and/or the tumor bed. Response or resistance and/or benefit may also be assessed by comparing time to distant metastasis or death of a patient following neoadjuvant or adjuvant non-chemotherapy and/or chemotherapy with time to distant metastasis or death of a patient not treated with non-chemotherapy and/or chemotherapy.

In one embodiment, the response or resistance and/or benefit of the subject is the disease free survival (DFS). In a preferred embodiment, the DFS may be selected from the list consisting of the pathological complete response (pCR); ypT (with levels ypT0, ypTis, ypT1, ypT2, ypT3, ypT4), ypT0 (with levels ypT0 vs. ypT+); ypT0 is (with levels ypT0/is vs. ypT+); ypN (with levels ypN0, ypN1, ypN2, ypN3); ypN0 (with levels ypN0 vs. ypN+); clinical response; loco-regional recurrence free interval (LRRFI); loco-regional invasive recurrence free interval (LRIRFI); distant-disease-free survival (DDFS); invasive disease-free survival (IDFS); event free survival (EFS) and/or overall survival (OS).

As used herein, the terms “pCR” and “pathological complete response” are used interchangeably and are well understood by the person skilled in the art. In particular, the terms “pCR” or “pathological complete response” may refer to ypT0 and ypN0, or ypT0 or ypTis and ypN0.

As used herein, ypT may be with levels ypT0, ypTis, ypT1, ypT2, ypT3, ypT4; ypT0 may be with levels ypT0 vs. ypT+; ypT0 is may be with levels ypT0/is vs. ypT+; ypN may be with levels ypN0, ypN1, ypN2, ypN3; ypN0 may be with levels ypN0 vs. ypN+.

As used herein, the term “clinical response” is well understood by the person skilled in the art and may include clinical response with levels of complete response, partial response, stable disease, progressive disease.

As used herein, the term “outcome” refers to a condition attained in the course of a disease. This disease outcome may e.g. be a clinical condition such as “recurrence of disease”, “development of metastasis”, “development of nodal metastasis”, “development of distant metastasis”, “survival”, “death”, “tumor remission rate”, a disease stage or grade or the like. In one embodiment, the outcome is the pathological complete response (pCR), loco-regional recurrence free interval (LRRFI), loco-regional invasive recurrence free interval (LRIRFI), distant-disease-free survival (DDFS), invasive disease-free survival (IDFS), event free survival (EFS) and/or overall survival (OS).

In one embodiment, the response and/or benefit and/or outcome may be the pCR. As used herein, the term “pathological complete response” (pCR) refers to a complete disappearance or absence of invasive tumor cells in the breast and/or lymph nodes as assessed by a histopathological examination.

Typically, said expression level of the at least one marker is compared to a reference level. Such “reference-value” can be a numerical cutoff value, it can be derived from a reference measurement of one or more other marker in the same sample, or one or more other marker and/or the same marker in one other sample or in a plurality of other samples. In one embodiment, the method comprises comparing the expression level of each of said at least one marker to a predetermined reference level.

The response or resistance to and/or the benefit from a treatment with a cancer immunotherapy in a subject suffering from or being at risk of development of a neoplastic disease, in particular breast cancer, may be predicted based on the comparison of the expression level of the at least one marker with the reference level. In another embodiment, the outcome of a treatment with a cancer immunotherapy in a subject suffering from or being at risk of development of a neoplastic disease, in particular breast cancer, may be prognosticated based on the comparison of the expression level of the at least one marker with the reference level. In another embodiment, the response or resistance to and/or the benefit from a treatment with a cancer immunotherapy in a subject suffering from or being at risk of development of a neoplastic disease, in particular breast cancer, may be predicted and the outcome of a treatment with a cancer immunotherapy in a subject suffering from or being at risk of development of a neoplastic disease, in particular breast cancer, may be prognosticated based on the comparison of the expression level of the at least one marker with the reference level. Such a reference level can e.g. be predetermined level that has been determined based on a population of healthy subjects. In one embodiment, the reference level comprises the expression level of the at least one marker in a sample obtained from at least one healthy subject, preferably the mean expression level of the at least one marker in samples obtained from a healthy population.

The reference value may be lower or higher than the expression level of the at least one marker. For example, the reference value may be 2-fold lower or 2-fold higher than the expression level of the at least one marker. The difference between the expression level of the at least one marker compared to the reference value may alternatively be determined by absolute values, e.g. by the difference of the expression level of the at least one marker and the reference value, or by relative values, e.g. by the percentage increase or decrease of the expression level of the at least one marker compared to the reference value. The expression level of the at least one marker which deviates from the reference value may be indicative for a particular response and/or benefit and/or outcome of a treatment with cancer immunotherapy in a subject suffering from or being at risk of development of a neoplastic disease, in particular breast cancer. In other words, an upregulation or a downregulation of the expression level of the at least one marker compared to the reference value may be indicative for a response and/or benefit and/or good outcome from a treatment with a cancer immunotherapy in said subject. In another embodiment, an upregulation or a downregulation of the expression level of the at least one marker compared to the reference value may be indicative for a non-response and/or no benefit and/or adverse outcome from a treatment with an immune checkpoint inhibitor in said subject. In particular, the extent of upregulation or a downregulation of the expression level of the at least one marker compared to the reference value may be indicative for a particular response and/or benefit and/or outcome of a treatment with cancer immunotherapy in a subject suffering from or being at risk of development of a neoplastic disease, in particular breast cancer. For example, the expression level of the at least one marker above by 3-fold rather than above 2-fold compared to the reference value may be indicative with a higher likelihood for a response and/or benefit from a treatment with a cancer immunotherapy in said subject.

In one embodiment, the comparison of the expression level of the at least one marker to the reference value indicates the likelihood of the subject for a response and/or benefit of a treatment with the cancer immuotherapy. In another embodiment, the comparison of the expression level of the at least one marker to the reference value indicates the likelihood of the subject for an outcome of a treatment with the cancer immunotherapy. In another embodiment, the comparison of the expression level of the at least one marker to the reference value indicates the likelihood of the subject for a response and/or benefit of a treatment with the cancer immuotherapy and/or the likelihood of the subject for an outcome of a treatment with the immunotherapy.

In one embodiment, an expression level of the at least one marker above said reference level in the sample is indicative for a response and/or benefit from a treatment with a cancer immunotherapy in said subject. In another embodiment, an expression level of the at least one marker above said reference level in the sample is indicative for a positive outcome of a treatment with a cancer immunotherapy in said subject. In another embodiment, an expression level of the at least one marker above said reference level in the sample is indicative for a response and/or benefit from a treatment with a cancer immunotherapy in said subject and for a positive outcome of a treatment with a cancer immunotherapy in said subject.

In one embodiment, an expression level of the at least one marker below said reference level in the sample is indicative for a response and/or benefit from a treatment with a cancer immunotherapy in said subject. In another embodiment, an expression level of the at least one marker below said reference level in the sample is indicative for a positive outcome of a treatment with a cancer immunotherapy in said subject. In another embodiment, an expression level of the at least one marker below said reference level in the sample is indicative for a response and/or benefit from a treatment with a cancer immunotherapy in said subject and for a positive outcome of a treatment with a cancer immunotherapy in said subject.

The skilled artisan will understand that associating a diagnostic or prognostic indicator, i.e. the expression level of the at least one marker, with the prediction of a response, benefit or with a prognostic risk of a future clinical outcome is a statistical analysis. For example, a marker level of lower than X may signal that a subject is more likely to suffer from an adverse outcome than a subject with a level more than or equal to X, as determined by a level of statistical significance. Additionally, a change in marker concentration from baseline levels may be reflective of subject prognosis, and the degree of change in marker level may be related to the severity of adverse events. Statistical significance is often determined by comparing two or more populations, and determining a confidence interval and/or a p value; see, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001. For example, the expression level of the at least one marker is indicative for the prediction and/or said prognosis and/or outcome compared to the expression level of a reference value at a p-value equal or below 0.005, preferably 0.001, more preferably 0.0001 and even more preferably below 0.0001.

The present invention also relates to the use of the method for predicting a response or resistance to and/or a benefit from a treatment with a cancer immunotherapy in a subject suffering from or being at risk of developing a neoplastic disease. Equally, the present invention relates to the use of the method for predicting the outcome of a treatment with a cancer immunotherapy in a subject suffering from or being at risk of developing a neoplastic disease.

In addition to the expression level of the at least one marker, further parameters of the subject may be determined. As used herein, a parameter is a characteristic, feature, or measurable factor that can help in defining a particular system. A parameter is an important element for health- and physiology-related assessments, such as a disease/disorder/clinical condition risk. Furthermore, a parameter is defined as a characteristic that is objectively measured and evaluated as an indicator of normal biological processes, pathogenic processes, or pharmacologic responses to a therapeutic intervention. For example, such further markers include but are not limited to age, sex, menopausal status, molecular subtype, estrogen-receptor (ER) status, progesterone-receptor (PR) status, human epidermal growth factor receptor 2 (HER2) status, Ki-67, tumor infiltrating lymphocytes, PD-1 activity, PD-L1 activity, histological tumor type, nodal status, metastases status, TNM staging, smoking history, ECOG performance status, Karnofsky status, tumor size at baseline and/or tumor grade at baseline. However, the method of the present invention does not need to rely on further parameters. In one embodiment, the method further comprises the determination of one more clinical parameters selected from the group consisting of pathological grading of the tumor, tumor size and nodal status. For example, the clinical parameter may be the pathological grading of the tumor at baseline and/or the tumor size at baseline and/or nodal status at baseline. The baseline refers to a value representing an initial level of a measurable quantity. The person skilled in the art knows that the baseline level may be determined before subject(s) are exposed to an environmental stimulus, receive an intervention such as a therapeutic treatment, or before a change of an environmental stimulus or a change in intervention such as a change in therapeutic treatment is induced. For example, the baseline may be determined before the start of the treatment of the subject(s) or before the start of a therapeutic intervention, such as an immunotherapy, or before the start of another therapeutic intervention, such as a non-chemotherapy or chemotherapy combined with an immunotherapy. The baseline level may be used for comparison with values representing response or resistance, benefit and/or outcome to an environmental stimulus and/or intervention, for example a particular treatment.

In another embodiment the sample obtained from the subject is taken after one or more applications of an immune checkpoint inhibitor.

In another embodiment samples are obtained from the subject at baseline and after one or more applications of an immune checkpoint inhibitor, and the dynamic change of one or more biomarkers is calculated as difference or ratio between the biomarkers after immune checkpoint inhibitor application and the biomarkers at baseline. As for example, the expression level of the at least one marker determined in a sample obtained from the subject taken after one or more applications of an immune checkpoint inhibitor or obtained from the subject at baseline and after one or more applications of an immune checkpoint inhibitor is selected from the group consisting of markers as identified in Table 10.1, preferably as identified in Table 10.2.

In another embodiment, in the sample obtained from said subject the expression levels of at least two, at least three, at least four, at least five, at least ten, at least twenty markers related to immune response and/or a marker selected from the group consisting of the markers as identified in Table 6.1, Table 7, Table 8.1, Table 2.1, Table 3.1, Table 4.1, Table 5.1 and Table 10.1 are determined.

In one embodiment, the method comprises determining a score based on
(i) the expression levels of the at least two, at least three, at least four, at least five, at least ten, at least twenty markers; or
(ii) the expression level of the at least one marker and the at least one clinical parameter.

In one embodiment, the method of the invention relates to determining the expression level of the at least one marker,
(a) wherein the at least one marker is selected from the group of the markers as identified in Table 2.1, preferably in Table 2.2, more preferably in Table 2.3, more preferably in Table 2.4, more preferably in Table 2.5, more preferably in Table 2.6, more preferably in Table 2.7, more preferably in Table 2.8, more preferably in Table 2.9, more preferably in Table 2.10, more preferably in Table 2.11 and even more preferably in Table 2.12; and/or
(b) wherein the at least one marker is selected from the group of the markers as identified in Table 3.1, preferably in Table 3.2, more preferably in Table 3.3, more preferably in Table 3.4, more preferably in Table 3.5, more preferably in Table 3.6, more preferably in Table 3.7, more preferably in Table 3.8, more preferably in Table 3.9, more preferably in Table 3.10, more preferably in Table 3.11 and even more preferably in Table 3.12; and/or
(c) wherein the at least one marker is selected from the group of the markers as identified in Table 4.1, preferably in Table 4.2, more preferably in Table 4.3, more preferably in Table 4.4, more preferably in Table 4.5, more preferably in Table 4.6, more preferably in Table 4.7, more preferably in Table 4.8, more preferably in Table 4.9, more preferably in Table 4.10, more preferably in Table 4.11 and even more preferably in Table 4.12; and/or
(d) wherein the at least one marker is selected from the group of the markers as identified in Table 5.1, preferably in Table 5.2, more preferably in Table 5.3, more preferably in Table 5.4, more preferably in Table 5.5, more preferably in Table 5.6, more preferably in Table 5.7, more preferably in Table 5.8, more preferably in Table 5.9, more preferably in Table 5.10, more preferably in Table 5.11 and even more preferably in Table 5.12; and/or
(e) wherein the at least one marker is selected from the group of the markers as identified in Table 6.1, preferably in Table 6.2, more preferably in Table 6.3, more preferably in Table 6.4, more preferably in Table 6.5, more preferably in Table 6.6, more preferably in Table 6.7, more preferably in Table 6.8, more preferably in Table 6.9, more preferably in Table 6.10, more preferably in Table 6.11 and even more preferably in Table 6.12; and/or
(f) wherein the at least one marker is selected from the group of the markers as identified in Table 7; and/or
(g) wherein the at least one marker is selected from the group of the markers as identified in Table 8.1, preferably in Table 8.2, more preferably in Table 8.3, more preferably in Table 8.4, more preferably in Table 8.5, more preferably in Table 8.6, more preferably in Table 8.7, more preferably in Table 8.8, more preferably in Table 8.9, more preferably in Table 8.10, more preferably in Table 8.11 and even more preferably in Table 8.12; is determined.

The at least one marker may be selected from the same group or from different groups according to a) to g). In one embodiment, the markers may be selected from the same group of groups a) to g). In another embodiment, the markers may be selected from different groups of groups a) to g). For example, the marker may be selected from one of groups e) to g). As another example, the marker may be selected from different groups of groups e) to g).

As used herein, the term “score” refers to a numeric value derived from the combination of the expression level of at least two markers and/or the combination of the expression level of the at least one marker and at least one further parameter. As used herein, the term “combination” or “combining” refers to deriving a numeric value from a determined expression level of at least two marker, or from a determined expression level of at least one marker and at least one further parameter. An algorithm may be applied to one or more expression level of at least two marker or the expression level of at least one marker and at least one further parameter to obtain the numerical value or the score. An “algorithm” is a process that performs some sequence of operations to produce information.

Combining these expression levels and/or parameters can be accomplished for example by multiplying each expression level and/or parameter with a defined and specified coefficient and summing up such products to yield a score. The score may be also derived from expression levels together with further parameter(s) like lymph node status or tumor grading as such variables can also be coded as numbers in an equation. The score may be used on a continuous scale to predict the response or resistance and/or benefit and/or the outcome of the subject to the treatment with an immune checkpoint inhibitor. Cut-off values may be applied to distinguish clinical relevant subgroups, i.e. “responder”, “non-responder”, “positive outcome” and “negative outcome”.

Cutoff values for such scores can be determined in the same way as cut-off values for conventional diagnostic markers and are well known to those skilled in the art. For example, one way of determining such cut-off value is to construct a receiver-operator curve (ROC curve) on the basis of all conceivable cut-off values, determining the single point on the ROC curve with the lowest proximity to the upper left corner (0/1) in the ROC plot. Typically, most of the time cut-off values will be determined by less formalized procedures by choosing the combination of sensitivity and specify determined by such cut-off value providing the most beneficial medical information to the problem investigated.

A “discriminant function” is a function of a set of variables used to classify an object or event. A discriminant function thus allows classification of a patient, samples or event into a category or a plurality of categories according to data or parameters available from said subject, sample or event. Such classification is a standard instrument of statistical analysis well known to the skilled person. For example, the subject may be classified to be indicative for the prediction and/or prognosis of group i) to iv):
i) an increased likelihood of the patient to respond and/or benefit from a cancer immunotherapy treatment;
ii) an increased likelihood of the patient not to respond and/or benefit to a cancer immunotherapy treatment;
iii) an increased likelihood of the patient to have a positive outcome to a cancer immunotherapy treatment;
iv) an increased likelihood of the patient have a negative outcome to a cancer immunotherapy treatment.

Classification is not limited to these categories, but may also be performed into a plurality of categories, such as “responder” and “good outcome” or grading or the like. Classification shall also be understood in a wider sense as a discriminating score, where e.g. a higher score represents a higher likelihood of distant metastasis, e.g. the (overall) risk of a distant metastasis. Examples for discriminant functions which allow a classification include, but are not limited to functions defined by support vector machines (SVM), k-nearest neighbors (INN), (naive) Bayes models, linear regression models or piecewise defined functions such as, for example, in subgroup discovery, in decision trees, in logical analysis of data (LAD) and the like. In a wider sense, continuous score values of mathematical methods or algorithms, such as correlation coefficients, projections, support vector machine scores, other similarity-based methods, combinations of these and the like are examples for illustrative purpose. For example, the expression level of each of said at least one marker comprises combining the expression level of each of the at least one marker with a coefficient, wherein the coefficient is indicative for the prognosis and/or prediction.

In one embodiment, the at least one marker is substituted by at least one substitute marker, wherein the expression level of the substitute marker correlates with the expression level of the at least one marker. The decision whether the at least one marker may be substitute with a substitute marker may be determined by the Pearson correlation coefficient. The application of Pearson's correlation coefficient is common to statistical sampling methods, and it may be used to determine the correlation of two variables. The Pearson coefficient may vary between −1 and +1. A coefficient of 0 indicates that neither of the two variables can be predicted from the other by a linear equation, while a correlation of +1 or −1 indicates that one variable may be perfectly predicted by a linear function of the other. A more detailed discussion of the Pearson coefficient may be found in McGraw-Hill Encyclopedia of Science and Technology, 6th Edition, Vol. 17. For example, the substitute marker correlates with the at least one marker by an absolute value of the Pearson correlation coefficient of at least 10.41, preferably at least 10.71, more preferably of at least 10.81. Some useful substitute marker substitutions are listed in Table 30, below.

The present invention also relates to kits and the use of kits for assessing the likelihood whether a patient suffering from or at risk of developing a neoplastic disease, in particular breast cancer, will benefit from and/or respond to or be resistant to a cancer immunotherapy treatment. The kit may comprise one or more detection reagents for determining the level of the expression level of the at least one marker and reference data including the reference level of the at least one marker, optionally wherein said detection reagents comprise at least a pair of oligonucleotides capable of specifically binding to the at least one marker. As used herein, the term “primer” refers to the ordinary meaning of this term which is well known to the person skilled in the art of molecular biology. Primers shall be understood as being polynucleotide molecules having a sequence identical, complementary, homologous, or homologous to the complement of the regions of a target molecule, which is to be detected or quantified, e.g. the at least one marker.

In a particularly preferred embodiment of the methods of the present invention, said cancer immunotherapy is an immune checkpoint inhibitor therapy (preferably durvalumab, more preferably durvalumab in combination with nab-paclitaxel followed by dose-dense epirubicin plus cyclophosphamid (EC)) and the neoplastic disease is breast cancer. In this context, the sample is preferably an FFPE sample of the tumor and mRNA expression of the genes is preferably determined using a microarray. Further in this context, the end-point is preferably pCR, more preferably no invasive and no-non invasive tumor residuals in breast and in axillary lymph nodes. Further, in this context a panel of at least two markers is preferably determined, more preferably the combinations listed in Tables 9.1 to 9.34 or Tables 17 to 28.

Particularly preferred markers in the context of all aspects and embodiments of the methods of the present invention are, for example, PSIP1, TAP1, THBS4, HLA_B, HLA_A, GNLY, ETV7, RUNX1, ADAMTS1, IRF2 and IL6R. In one embodiment, the expression level of at least one marker selected from the group consisting of PSIP1, TAP1, THBS4, HLA_B, HLA_A, GNLY, ETV7, RUNX1, ADAMTS1, IRF2 and IL6R is determined. In another embodiment the expression level of at least one marker selected from the group consisting of PSIP1, TAP1, THBS4, GNLY, ETV7, RUNX1, ADAMTS1 and IRF2 is determined. In yet another embodiment, the expression level of at least one marker selected from the group consisting of RUNX1, ADAMTS1, PSIP1, TAP1 and THBS4 is determined. In yet another embodiment, the expression level of at least one marker selected from the group consisting of THBS4, HLA_B, HLA_A, GNLY, ETV7, RUNX1, ADAMTS1, IRF2 and IL6R is determined. In yet another embodiment, the expression level of at least one marker selected from the group consisting of PSIP1, TAP1, HLA_B, HLA_A, GNLY, ETV7, RUNX1, ADAMTS1 and IRF2 is determined.

All patent and non-patent documents cited herein are hereby incorporated by reference in their entirety.

### EXAMPLES

### Example 1: Overview of Clinical Study

A randomized double blind placebo controlled phase II trial investigating the pCR rate of neoadjuvant chemotherapy including nab-paclitaxel followed by dose-dense epirubicin+cyclophosphamid (EC) with durvalumab vs. placebo in breast cancer was carried out.

Durvalumab or placebo was given every 4 weeks (in addition to nab-paclitaxel followed by standard EC). Some patients participated in the window phase, wherein durvalumab/placebo alone was given two weeks prior to start of nab-paclitaxel followed by a biopsy.

The primary objective was the comparison of proportions of patients who achieved a pathological complete response (ypT0/ypN0) after neoadjuvant treatment between arms. Secondary objectives were comparison of the following primary and secondary endpoints between treatment arms: The primary efficacy endpoint was pCR defined as no invasive and no-non invasive tumor residuals in breast and in axillary lymph nodes (ypT0/ypN0) after neoadjuvant therapy. Histopathological assessment was done at the local sites' pathology. All histopathological reports were centrally collected and evaluated by an independent pathologist (KE) blinded to treatment and not otherwise involved into the trial. Patients who had involved lymph nodes by sentinel node biopsy and did not undergo axillary surgery, were rated as non pCR irrespective of the response in the breast. Secondary pCR endpoints (ypT0is/ypN0) were assessed in the same way. Clinical response in the breast and nodes after durvalumab treatment and prior to surgery was assessed using preferably imaging response (priority sonography followed by MRI or mammography) or palpation, if missing. Toxicity reported as adverse events irrespective of relatedness to study treatment were based on NCI-CTC criteria v4.0.

Formalin-fixed paraffin-embedded (FFPE) samples of tumor tissue are used for extraction of nucleic acids. RNA expression of the investigated genes was quantitatively determined using Targeted RNA Sequencing. In particular, Targeted RNA Sequencing was used for pre-therapeutic, FFPE core biopsies, which were evaluable for profiling of 2559 genes using the HTG EdgeSeq® system (HTG Oncology biomarker panel) that combines a nuclease protection assay with next generation sequencing. Data were processed as recommended by HTG, median normalized within each sample and across the experiment, and log 2-transformed. For differential gene expression analyses, data was scale-normalized and linear models were fit after filtering for minimal expression (>4) and variability (IQR>1).

### Example 1

Genes discriminating patients with pCR from patients without pCR in the durvalumab arm are prognostic. The following table shows genes that discriminate well according to a t-test. The left half of the table shows genes found by using the pCR endpoint defined as ypT0/ypN0, while the right half of the table shows genes found by using the pCR endpoint ypT0 is/ypN0. Columns “prognosis” contains “good” if a higher gene expression is related to a higher likelihood of a pCR and “bad” if a higher gene expression is related to a lower likelihood of a pCR. Columns “p” denotes the p-value from the t-test.

TABLE 11

| ypT0/ypN0 | | | ypT0is/ypN0 | | |
|---|---|---|---|---|---|
| gene | prognosis | p | gene | prognosis | p |
| PSIP1 | good | <.0001 | TAP1 | good | <.0001 |
| TAP1 | good | <.0001 | CD38 | good | <.0001 |
| HLA_B | good | <.0001 | THBS4 | bad | <.0001 |
| GBP1 | good | 0.0001 | ETV7 | good | <.0001 |
| HLA_A | good | 0.0001 | LAG3 | good | 0.0001 |
| THBS4 | bad | 0.0002 | CD274 | good | 0.0001 |
| STAT1 | good | 0.0002 | TIMP3 | bad | 0.0001 |
| ITGA2 | bad | 0.0003 | IRF2 | good | 0.0001 |
| TIMP3 | bad | 0.0003 | COL1A1 | bad | 0.0002 |
| CXCL10 | good | 0.0004 | IL6R | good | 0.0002 |
| TAP2 | good | 0.0005 | GNLY | good | 0.0002 |
| JAK2 | good | 0.0005 | ITGA2 | bad | 0.0002 |
| CD38 | good | 0.0006 | IRF7 | good | 0.0002 |
| ETV7 | good | 0.0006 | PLAT | bad | 0.0003 |
| LAG3 | good | 0.0007 | PSIP1 | good | 0.0003 |
| IRF9 | good | 0.0008 | HLA_B | good | 0.0003 |
| IRF2 | good | 0.0009 | TAP2 | good | 0.0003 |
| GNLY | good | 0.0010 | STAT1 | good | 0.0004 |
| PDCD1LG2 | good | 0.0011 | DHX58 | good | 0.0004 |
| BOK | bad | 0.0012 | HLA_A | good | 0.0004 |
| IRS1 | bad | 0.0013 | COL1A2 | bad | 0.0004 |
| DDX58 | good | 0.0013 | GBP1 | good | 0.0004 |
| IGFBP7 | bad | 0.0015 | DDX58 | good | 0.0005 |
| COL1A1 | bad | 0.0015 | CXCL10 | good | 0.0005 |
| HEY2 | bad | 0.0016 | CCL7 | good | 0.0006 |
| DHX58 | good | 0.0018 | MX1 | good | 0.0006 |
| IRF7 | good | 0.0018 | PDCD1LG2 | good | 0.0006 |
| PLAT | bad | 0.0019 | JAK2 | good | 0.0006 |
| SPARC | bad | 0.0023 | TIFA | good | 0.0007 |
| MX1 | good | 0.0025 | AK3 | good | 0.0010 |
| CD274 | good | 0.0026 | PMEPA1 | bad | 0.0010 |
| HIST1H3H | good | 0.0027 | CD55 | bad | 0.0010 |
| IFI27 | good | 0.0028 | COL3A1 | bad | 0.0011 |
| NOTCH4 | bad | 0.0031 | THBS2 | bad | 0.0012 |
| KDR | bad | 0.0031 | COL5A1 | bad | 0.0013 |
| COL1A2 | bad | 0.0032 | SLAMF7 | good | 0.0013 |
| SPRY4 | bad | 0.0034 | CD83 | good | 0.0014 |
| IL6R | good | 0.0035 | BOK | bad | 0.0014 |
| SLAMF7 | good | 0.0036 | INHBA | bad | 0.0015 |
| EGFR | bad | 0.0037 | DNAJB2 | bad | 0.0015 |
| CXCL13 | good | 0.0042 | LOX | bad | 0.0016 |
| DLL4 | bad | 0.0042 | CD79A | good | 0.0018 |
| ISG15 | good | 0.0043 | PPP2CB | bad | 0.0018 |
| EDIL3 | bad | 0.0047 | EAF2 | good | 0.0019 |
| TIFA | good | 0.0048 | SFRP2 | bad | 0.0020 |
| CAV2 | bad | 0.0051 | TLR3 | good | 0.0020 |
| COL3A1 | bad | 0.0051 | IFI27 | good | 0.0021 |
| CDKN2A | good | 0.0051 | IGFBP7 | bad | 0.0022 |
| TLR3 | good | 0.0051 | RAC3 | bad | 0.0022 |
| CAV1 | bad | 0.0056 | IRF9 | good | 0.0025 |

According to the table above the most significant gene for ypT0/ypN0 is PSIP1, for ypT0is/ypN0 it is TAP1; both are “good” prognosis genes. The best “bad” prognosis gene is THBS4 for both endpoints. One can apply cutoffs to the gene expression (here the expression means from the whole cohort are used) to classify patients into high and low expressers and to determine the pCR rates in the respective subgroups. The following table shows the pCR rates in the durvalumab arm:

TABLE 12

| | | | pCR rate if | pCR rate if |
|---|---|---|---|---|
| gene | cutoff | pCR definition | expression high | expression low |
| PSIP1 | 9.47 | ypT0/ypN0 | 77% | 38% |
| TAP1 | 9.92 | ypT0is/ypN0 | 79% | 42% |
| THBS4 | 7.16 | ypT0/ypN0 | 39% | 71% |
| THBS4 | 7.16 | ypT0is/ypN0 | 43% | 76% |

### Example 2

Same as Example 1, but based on Wilcoxon tests instead of t-tests.

TABLE 13

| ypT0/ypN0 | | | ypT0is/ypN0 | | |
|---|---|---|---|---|---|
| gene | prognosis | p | gene | prognosis | p |
| PSIP1 | good | <.0001 | TAP1 | good | <.0001 |
| TAP1 | good | <.0001 | RUNX1 | bad | <.0001 |
| HLA_B | good | <.0001 | ETV7 | good | <.0001 |
| THBS4 | bad | 0.0001 | THBS4 | bad | <.0001 |
| ETV7 | good | 0.0002 | CD38 | good | <.0001 |
| HLA_A | good | 0.0002 | GNLY | good | 0.0001 |
| GBP1 | good | 0.0002 | CD274 | good | 0.0001 |
| RUNX1 | bad | 0.0003 | COL1A1 | bad | 0.0002 |
| ITGA2 | bad | 0.0004 | HLA_B | good | 0.0002 |
| TIMP3 | bad | 0.0004 | IRF7 | good | 0.0002 |
| CXCL10 | good | 0.0005 | TIMP3 | bad | 0.0002 |
| GNLY | good | 0.0005 | LAG3 | good | 0.0002 |
| PDCD1LG2 | good | 0.0005 | IRF2 | good | 0.0002 |
| STAT1 | good | 0.0007 | PSIP1 | good | 0.0003 |
| CD38 | good | 0.0007 | IL6R | good | 0.0003 |
| TAP2 | good | 0.0007 | PLAT | bad | 0.0003 |
| NOTCH4 | bad | 0.0008 | CD55 | bad | 0.0004 |
| IRF9 | good | 0.0008 | PDCD1LG2 | good | 0.0004 |
| LAG3 | good | 0.0008 | ITGA2 | bad | 0.0005 |
| HIST1H3H | good | 0.0009 | TIFA | good | 0.0005 |
| JAK2 | good | 0.0010 | COL1A2 | bad | 0.0005 |
| IRF2 | good | 0.0011 | HLA_A | good | 0.0006 |
| CXCL13 | good | 0.0012 | TAP2 | good | 0.0006 |
| KNTC1 | good | 0.0012 | DHX58 | good | 0.0006 |
| AHNAK | bad | 0.0014 | GBP1 | good | 0.0007 |
| HEY2 | bad | 0.0015 | SLAMF7 | good | 0.0007 |
| BOK | bad | 0.0015 | CXCL10 | good | 0.0007 |
| IRF7 | good | 0.0016 | DDX58 | good | 0.0008 |
| DLL4 | bad | 0.0016 | AK3 | good | 0.0008 |
| COL1A1 | bad | 0.0018 | IRF1 | good | 0.0008 |
| DDX58 | good | 0.0020 | STAT1 | good | 0.0009 |
| IGFBP7 | bad | 0.0020 | THBS2 | bad | 0.0009 |
| VEGFB | bad | 0.0022 | JAK2 | good | 0.0010 |
| CDKN2A | good | 0.0025 | CD86 | good | 0.0010 |
| SPARC | bad | 0.0025 | COL3A1 | bad | 0.0011 |
| PLAT | bad | 0.0026 | DNAJB2 | bad | 0.0011 |
| IRF1 | good | 0.0027 | CD83 | good | 0.0011 |
| KDR | bad | 0.0027 | BOK | bad | 0.0012 |
| CD55 | bad | 0.0030 | IRF4 | good | 0.0012 |
| SLAMF7 | good | 0.0030 | CXCL13 | good | 0.0013 |
| CD274 | good | 0.0030 | RAC3 | bad | 0.0013 |
| DHX58 | good | 0.0032 | PPP2CB | bad | 0.0014 |
| MX1 | good | 0.0035 | SFRP2 | bad | 0.0014 |
| KDM1A | good | 0.0037 | VEGFB | bad | 0.0014 |
| EGER | bad | 0.0038 | CD79A | good | 0.0015 |
| GSN | bad | 0.0040 | MX1 | good | 0.0015 |
| IFI27 | good | 0.0040 | IRF9 | good | 0.0016 |
| IL6R | good | 0.0045 | COL5A1 | bad | 0.0017 |
| COL3A1 | bad | 0.0047 | HERPUD1 | good | 0.0017 |
| DNAJB2 | bad | 0.0047 | CCL7 | good | 0.0018 |

### Example 3

Same as Example 1, but based on Kolmogorov-Smirnov tests instead of t-tests.

TABLE 14

| ypT0/ypN0 | | | ypT0is/ypN0 | | |
|---|---|---|---|---|---|
| gene | prognosis | p | gene | prognosis | p |
| ETV7 | good | <.0001 | GNLY | good | <.0001 |
| GNLY | good | <.0001 | ETV7 | good | <.0001 |
| PSIP1 | good | <.0001 | RUNX1 | bad | <.0001 |
| TAP1 | good | 0.0002 | TIFA | good | 0.0002 |
| CDKN2A | good | 0.0004 | IRF7 | good | 0.0002 |
| RUNX1 | bad | 0.0006 | TAP1 | good | 0.0002 |
| MCM6 | good | 0.0007 | LAG3 | good | 0.0002 |
| KNTC1 | good | 0.0008 | COL1A1 | bad | 0.0002 |
| SPARC | bad | 0.0010 | CD38 | good | 0.0003 |
| IRF7 | good | 0.0011 | TNFRSF17 | good | 0.0004 |
| FGF13 | bad | 0.0011 | PLAT | bad | 0.0005 |
| JAK2 | good | 0.0012 | COL1A2 | bad | 0.0005 |
| THBS4 | bad | 0.0012 | IFNA2 | good | 0.0006 |
| HEY2 | bad | 0.0013 | JAK2 | good | 0.0006 |
| SHC2 | bad | 0.0014 | THBS4 | bad | 0.0007 |
| DLL4 | bad | 0.0016 | IRF4 | good | 0.0007 |
| AHNAK | bad | 0.0022 | TAP2 | good | 0.0007 |
| LAG3 | good | 0.0022 | MTHFD1 | good | 0.0007 |
| DLGAP5 | good | 0.0023 | IL6R | good | 0.0008 |
| PLAT | bad | 0.0024 | S100A6 | bad | 0.0010 |
| MSL2 | good | 0.0025 | CD274 | good | 0.0010 |
| HIST1H3H | good | 0.0025 | FGF13 | bad | 0.0010 |
| HLA_B | good | 0.0025 | COL5A2 | bad | 0.0010 |
| TAP2 | good | 0.0025 | RAC3 | bad | 0.0010 |
| GBP1 | good | 0.0032 | DLGAP5 | good | 0.0010 |
| JAG1 | bad | 0.0034 | COL5A1 | bad | 0.0011 |
| ITGA2 | bad | 0.0035 | TIMP3 | bad | 0.0013 |
| IRF9 | good | 0.0036 | SRM | good | 0.0013 |
| TIMP3 | bad | 0.0036 | PDGFB | bad | 0.0014 |
| RAC3 | bad | 0.0039 | CD83 | good | 0.0015 |
| BCL2A1 | good | 0.0042 | DNAJB2 | bad | 0.0017 |
| MAD2L1 | good | 0.0042 | BCL2A1 | good | 0.0018 |
| TNFRSF17 | good | 0.0042 | SLAMF7 | good | 0.0020 |
| FBXO5 | good | 0.0042 | CD79A | good | 0.0021 |
| MTHFD1 | good | 0.0044 | MAD2L1 | good | 0.0021 |
| VEGFB | bad | 0.0044 | MSH3 | good | 0.0021 |
| IGFBP7 | bad | 0.0047 | DLL4 | bad | 0.0022 |
| ACTA2 | bad | 0.0050 | COL3A1 | bad | 0.0023 |
| CXCL10 | good | 0.0050 | PSIP1 | good | 0.0023 |
| HLA_A | good | 0.0053 | GZMB | good | 0.0023 |
| KDM1A | good | 0.0053 | IGFBP7 | bad | 0.0024 |
| CD86 | good | 0.0056 | CD55 | bad | 0.0025 |
| HMOX1 | good | 0.0057 | SPARC | bad | 0.0025 |
| COL1A1 | bad | 0.0060 | XBP1 | good | 0.0025 |
| IFNA2 | good | 0.0060 | CDC7 | good | 0.0026 |
| CD38 | good | 0.0061 | HEY2 | bad | 0.0026 |
| NASP | good | 0.0061 | FN1 | bad | 0.0026 |
| BOK | bad | 0.0062 | SFRP2 | bad | 0.0029 |
| TIFA | good | 0.0066 | VEGFB | bad | 0.0029 |
| SLC25A13 | bad | 0.0068 | CD86 | good | 0.0029 |

### Example 4

A gene showing a statistical interaction between the gene expression and the treatment arm (durvalumab versus placebo, both combined with chemo therapy) with respect to pCR is predictive and may be used to decide whether durvalumab is beneficial for the patient or not. The following table contains the results of logistic regression models:
The dependent variable is either pCR defined as ypT0/ypN0 in the left half of the table or pCR defined as ypT0is/ypN0 in the right half of the table.
The independent variables are the treatment arm, the gene expression, and their interaction.

For each model four columns are reported:
Column “gene” contains the gene analyzed.
Column “odds ratio (placebo)” contains the unit odds ratio from the model for the placebo arm: It denotes the ratio of odds for pCR corresponding to an increase of the gene expression by one unit if the patient treated according to the placebo arm schema.
Column “odds ratio (durvalumab)” contains the respective odds ratio for a patient treated according to the durvalumab arm schema.
Column “p-value interaction” denotes the probability for the said two odds ratios to be statistically different (test for interaction).

If a gene is highly expressed the patient will benefit from the arm with higher odds ratio; if the gene is low expressed the patient will benefit from the arm with the lower odds ratio.

TABLE 15

| ypT0/ypN0 | | | | ypT0is/ypN0 | | | |
|---|---|---|---|---|---|---|---|
| | odds ratio | odds ratio | p-value | | odds ratio | odds ratio | p-value |
| gene | (placebo) | (durvalumab) | interaction | gene | (placebo) | (durvalumab) | interaction |
| ADAMTS1 | 2.033 | 0.538 | 0.0031 | RUNX1 | 1.018 | 0.176 | 0.0013 |
| RUNX1 | 0.965 | 0.261 | 0.0075 | IE6R | 0.843 | 4.508 | 0.0030 |
| MED12 | 4.998 | 0.328 | 0.0077 | DHX58 | 0.799 | 3.194 | 0.0031 |
| HEY2 | 1.100 | 0.569 | 0.0078 | COE1A1 | 1.076 | 0.434 | 0.0034 |
| IRF2 | 0.905 | 5.707 | 0.0082 | ADAMTS1 | 2.039 | 0.563 | 0.0040 |
| TMEM74B | 1.397 | 0.504 | 0.0088 | IRF2 | 0.972 | 8.091 | 0.0040 |
| PIK3CA | 4.181 | 0.615 | 0.0092 | GNEY | 0.931 | 1.951 | 0.0047 |
| HLA_A | 1.040 | 2.841 | 0.0095 | HLA_A | 0.917 | 2.548 | 0.0066 |
| GSN | 1.135 | 0.384 | 0.0141 | COE1A2 | 1.056 | 0.444 | 0.0068 |
| CCL28 | 1.183 | 0.728 | 0.0147 | CHI3E1 | 0.771 | 1.457 | 0.0078 |
| DHX58 | 0.887 | 2.612 | 0.0154 | PRKAA2 | 1.858 | 0.673 | 0.0101 |
| HLA_B | 1.194 | 2.984 | 0.0164 | QSOX2 | 0.527 | 3.247 | 0.0111 |
| IDH1 | 0.378 | 1.323 | 0.0180 | COE5A1 | 1.137 | 0.471 | 0.0113 |
| HRK | 1.634 | 0.731 | 0.0184 | HLA_B | 1.053 | 2.649 | 0.0119 |
| NKD1 | 1.353 | 0.677 | 0.0195 | RARB | 0.506 | 1.146 | 0.0129 |
| MADD | 0.853 | 7.533 | 0.0208 | SFRP2 | 1.041 | 0.537 | 0.0130 |
| PSIP1 | 1.572 | 5.773 | 0.0210 | ITPKB | 0.412 | 1.583 | 0.0137 |
| MAX | 0.860 | 7.883 | 0.0214 | MED12 | 4.907 | 0.412 | 0.0137 |
| PPID | 0.390 | 1.565 | 0.0218 | THBS4 | 0.859 | 0.427 | 0.0143 |
| ALKBH3 | 3.047 | 0.763 | 0.0221 | AK3 | 1.045 | 3.370 | 0.0145 |
| RAD51C | 3.929 | 0.910 | 0.0226 | MMP14 | 1.161 | 0.405 | 0.0151 |
| TLR3 | 0.857 | 2.499 | 0.0240 | EAF2 | 0.904 | 3.576 | 0.0154 |
| GPAT2 | 1.430 | 0.895 | 0.0243 | BCL2A1 | 0.862 | 1.970 | 0.0154 |
| TNFRSF8 | 1.773 | 0.819 | 0.0259 | PPID | 0.387 | 1.728 | 0.0155 |
| NERP3 | 1.709 | 0.593 | 0.0266 | DDX58 | 1.016 | 2.577 | 0.0157 |
| CXCE8 | 1.486 | 0.727 | 0.0267 | ACSL4 | 0.556 | 2.788 | 0.0159 |
| ECN2 | 1.108 | 0.837 | 0.0298 | HDAC8 | 0.432 | 1.900 | 0.0161 |
| PTPN11 | 2.324 | 0.393 | 0.0300 | HEY2 | 1.120 | 0.626 | 0.0164 |
| CCE17 | 1.325 | 0.724 | 0.0308 | LAG3 | 1.053 | 2.253 | 0.0167 |
| SEC45A3 | 1.121 | 0.558 | 0.0310 | COL3A1 | 1.019 | 0.495 | 0.0175 |
| CECF1 | 1.204 | 0.538 | 0.0311 | TADA3 | 2.497 | 0.603 | 0.0179 |
| MEET3 | 0.741 | 1.548 | 0.0314 | SOCS4 | 0.780 | 5.083 | 0.0192 |
| TNFAIP3 | 0.810 | 2.262 | 0.0315 | CD47 | 1.002 | 2.526 | 0.0192 |
| BID | 2.680 | 0.603 | 0.0321 | TIMP3 | 0.866 | 0.362 | 0.0205 |
| KDR | 0.949 | 0.325 | 0.0334 | JAK2 | 1.072 | 3.643 | 0.0214 |
| XRCC5 | 1.075 | 0.468 | 0.0336 | PLA2G4A | 0.477 | 1.149 | 0.0217 |
| NFKB1 | 0.975 | 5.472 | 0.0341 | TMEM74B | 1.341 | 0.565 | 0.0229 |
| TOP3A | 0.762 | 2.670 | 0.0343 | P4HB | 1.085 | 0.381 | 0.0235 |
| CEACAM3 | 1.296 | 0.808 | 0.0348 | MYBL1 | 0.744 | 1.317 | 0.0235 |
| PTCHD1 | 1.319 | 0.712 | 0.0349 | TAP2 | 1.113 | 3.167 | 0.0236 |
| SELE | 2.073 | 0.934 | 0.0352 | MAT2A | 0.449 | 2.274 | 0.0238 |
| TMEM45B | 1.136 | 0.688 | 0.0358 | CCL7 | 1.112 | 2.103 | 0.0239 |
| CRLF2 | 1.380 | 0.791 | 0.0360 | NSD1 | 3.568 | 0.618 | 0.0240 |
| SLC16A1 | 0.716 | 1.633 | 0.0363 | GSN | 1.167 | 0.443 | 0.0245 |
| CEBPB | 0.787 | 1.673 | 0.0370 | RASSF1 | 0.440 | 1.758 | 0.0251 |
| DIABLO | 4.043 | 0.998 | 0.0375 | RAD51C | 3.164 | 0.780 | 0.0259 |
| QSOX2 | 0.558 | 2.317 | 0.0383 | CD38 | 1.122 | 1.975 | 0.0263 |
| MAPK3 | 1.161 | 0.265 | 0.0387 | PSIP1 | 1.275 | 4.039 | 0.0266 |
| UBB | 0.691 | 2.190 | 0.0388 | CCL19 | 0.794 | 1.167 | 0.0274 |
| TADA3 | 1.866 | 0.574 | 0.0392 | KRT7 | 1.313 | 0.712 | 0.0274 |

According to the table above the most significant gene is ADAMTS1 for ypT0/ypN0 and RUNX1 for ypT0is/ypN0; both favor placebo if highly expressed and favor durvalumab if low expressed. The most significant genes favoring the other treatment, respectively, are IRF2 for ypT0/ypN0 and IL6R for ypT0is/ypN0. Application of cutoffs to the gene expression (here the expression means from the whole cohort are used) to classify patients into high and low expressers yields the following pCR rates in the respective subgroups:

TABLE 16

| | | | pCR rate in | pCR rate in | pCR rate in | pCR rate in |
|---|---|---|---|---|---|---|
| | | | durvalumab arm | durvalumab arm | placebo arm | placebo arm |
| | | | if expression | if expression | if expression | if expression |
| gene | cutoff | pCR definition | high | low | high | low |
| ADAMTS1 | 8.96 | ypT0/ypN0 | 46% | 61% | 54% | 40% |
| RUNX1 | 10.05 | ypT0is/ypN0 | 47% | 91% | 49% | 55% |
| IRF2 | 8.20 | ypT0/ypN0 | 74% | 45% | 56% | 44% |
| IL6R | 8.75 | ypT0is/ypN0 | 70% | 40% | 55% | 38% |

### Example 5

Prognostication can be improved by combining the expression levels of several prognostic genes by mathematical algorithms into a score. One type of realization for such a combination (which has the advantage of high robustness and therefore high performance and reliability) is to create committees consisting of members, where each member is a linear combination of the levels of one or more genes. Members are prognostic algorithms by their own, are independent from each other and can be combined by addition of their scores to build a committee, where the committee has higher prognostic performance than each member alone.

The table below gives examples for members called m1, m2 . . . consisting of two genes each, shown in column “member”. The coefficients were determined from the durvalumab arm by bivariate logistic regression with respect to the dependent variable pCR defined as ypT0/ypN0. Each gene is contained in at most one member; therefore members are independent from each other and can be combined. A committee can be built by choosing one or more members and by adding the scores of the chosen members: As an example, a committee consisting of members m1 and m2 calculates its prognostic score as follows:

$$Committee score = m 1 + m 2 = 2.4 \; 2 {6}^{*} PSIP 1 + 2.70 {7}^{*} S O C S 4 + 1.77 {1}^{*} TAP 1 - 1.03 {0}^{*} BATF$$

It is important to note that after the committee has been built the order of summands is arbitrary, so from a committee score one cannot reconstruct its members. In the example above the committee score could also be calculated as

committee score=−1.030*BATF+2.426*PSIP1+2.707*SOCS4+1.771*TAP1

which is mathematically equivalent. Nevertheless, BATF and PSIP1 have not been combined into a member.

It is also important to note that members do not have to be combined in the order as listed in the table. For example, m1+m3+m7 is also a prognostic committee score.

Column “member” shows the mathematical definition of the members. Column “AUC(member)” shows the area under the receiver operator characteristic curve (AUC under the ROC curve) with respect to the single member score and pCR. Column “AUC(cum.)” shows the AUC under the ROC curve for the exemplary committee consisting of the respective member and all previous members (i.e. the respective “cum.” committee score in the table row for m3 is m1+m2+m3).

TABLE 17

| | AUC(mem- | |
|---|---|---|
| member | ber) | AUC(cum.) |
| m1 = 2.426*PSIP1 + 2.707*SOCS4 | 0.8480 | 0.8480 |
| m2 = 1.771*TAP1 − 1.030*BATF | 0.8218 | 0.8989 |
| m3 = 1.442*HLA_B − 1.490*RB1 | 0.8289 | 0.9133 |
| m4 = 0.744*GBP1 − 0.682*THBS4 | 0.8020 | 0.9115 |
| m5 = 1.401*HLA_A + 1.713*TBL1X | 0.7919 | 0.9067 |
| m6 = 1.175*STAT1 + 0.563*CA9 | 0.7871 | 0.9031 |
| m7 = −0.753*ITGA2 − 0.877*TIMP3 | 0.7984 | 0.9097 |
| m8 = 0.664*CXCL10 + 1.400*KDM1A | 0.7959 | 0.9043 |
| m9 = 0.856*CD38 + 2.080*CASP8AP2 | 0.8236 | 0.9013 |
| m10 = 1.221*TAP2 + 0.957*DLGAP5 | 0.7955 | 0.8923 |
| m11 = 2.000*JAK2 − 2.140*ENG | 0.7766 | 0.8953 |
| m12 = 1.581*LAG3 − 1.622*CMKLR1 | 0.8038 | 0.8983 |
| m13 = 1.494*IRF9 − 1.245*DLL4 | 0.7721 | 0.8911 |
| m14 = 1.100*ETV7 − 0.959*TMEM74B | 0.7727 | 0.8911 |
| m15 = 2.451*IRF2 − 1.325*SLIT2 | 0.7889 | 0.8900 |
| m16 = 0.889*GNLY − 1.120*LFNG | 0.7906 | 0.8923 |
| m17 = −1.281*BOK − 1.247*NOTCH4 | 0.8116 | 0.8911 |
| m18 = 0.862*PDCD1LG2 − 0.947*IRS1 | 0.7708 | 0.8906 |
| m19 = 0.930*DDX58 + 1.222*MTHFD1 | 0.7585 | 0.8858 |
| m20 = 0.985*IRF7 + 1.210*EZH2 | 0.7784 | 0.8888 |
| m21 = −1.079*PLAT − 1.542*STK3 | 0.7661 | 0.8911 |
| m22 = −0.796*HEY2 + 1.816*RAD9A | 0.7799 | 0.8900 |
| m23 = −0.730*COL1A1 + 0.587*IFI27 | 0.7649 | 0.8864 |
| m24 = −1.668*IGFBP7 − 1.527*PRKCE | 0.7632 | 0.8894 |
| m25 = 1.259*DHX58 + 1.090*TTK | 0.7715 | 0.8876 |
| m26 = 0.548*MX1 − 1.089*KDR | 0.7515 | 0.8858 |
| m27 = −1.461*RUNX1 + 1.240*PML | 0.7889 | 0.8876 |
| m28 = 0.764*HIST1H3H + 0.658*CCL7 | 0.7637 | 0.8858 |
| m29 = −2.002*SPRY4 − 1.772*CSDE1 | 0.7690 | 0.8864 |
| m30 = −0.971*SPARC − 0.385*SPDEF | 0.7632 | 0.8876 |
| m31 = 1.116*CD274 − 0.830*TNXB | 0.7859 | 0.8888 |
| m32 = 0.732*SLAMF7 − 1.522*TGFBR2 | 0.7608 | 0.8906 |
| m33 = −0.798*COL1A2 + 0.946*PRDM1 | 0.7380 | 0.8882 |
| m34 = 0.579*ISG15 − 1.470*PPP2CB | 0.7240 | 0.8858 |
| m35 = 0.696*CCL4 + 0.550*CDKN2A | 0.7518 | 0.8846 |
| m36 = −1.167*EGFR − 2.384*MED12 | 0.7566 | 0.8894 |
| m37 = 0.740*CXCL13 − 1.018*FLT3 | 0.7572 | 0.8923 |
| m38 = 1.607*IL6R − 0.662*CCL14 | 0.7542 | 0.8882 |
| m39 = −1.305*CAV1 − 0.966*RAC3 | 0.7719 | 0.8923 |
| m40 = 1.896*TLR3 − 1.282*STEAP4 | 0.8044 | 0.8929 |
| m41 = −0.618*EDIL3 − 1.747*TOP1 | 0.7491 | 0.8894 |
| m42 = −0.738*ALDH1A3 + 2.496*MADD | 0.7554 | 0.8911 |
| m43 = 2.061*NFKB1 − 0.883*PTGR1 | 0.7177 | 0.8923 |
| m44 = −2.111*CAV2 − 0.358*FGF4 | 0.8183 | 0.9291 |
| m45 = 1.355*TIFA + 1.147*HLA_E | 0.7644 | 0.9268 |
| m46 = −1.721*MAPK3 + 1.780*CRK | 0.7422 | 0.9299 |
| m47 = −0.733*COL3A1 − 0.582*CXXC4 | 0.7462 | 0.9268 |
| m48 = −1.499*DNAJB2 − 0.953*TSPAN7 | 0.7554 | 0.9276 |
| m49 = 0.728*IDO1 + 1.956*ARID1A | 0.7661 | 0.9306 |
| m50 = 1.455*CD83 − 0.693*RELN | 0.7422 | 0.9314 |

According to the table above the first members have excellent AUCs. The following table contains examples of single members and committees where scores are dichotomized to classify patients from the durvalumab arm into low and high expression:

TABLE 18

| | | | pCR rate if | pCR rate if |
|---|---|---|---|---|
| algorithm | cutoff | pCR definition | expression high | expression low |
| m1 | 46.06 | ypT0/ypN0 | 85% | 26% |
| m2 | 10.27 | ypT0/ypN0 | 81% | 31% |
| m1 + m2 | 56.33 | ypT0/ypN0 | 91% | 26% |
| m1 + m2 + m3 | 64.90 | ypT0/ypN0 | 89% | 26% |

### Example 6

Same as Example 5, but with pCR defined as ypT0is/ypN0 (instead of ypT0/ypN0), three (instead of two) genes per member, and covariables grading and tumor size (instead of no covariables) when determining the logistic regression coefficients for each member.

TABLE 19

| | AUC(mem- | |
|---|---|---|
| member | ber) | AUC(cum.) |
| m1 = 1.121*TAP1 − 1.691*PLAT − 2.498*SRF | 0.8788 | 0.8788 |
| m2 = 1.791*CD38 − 2.770*RIPK3 − 1.296*RAC3 | 0.8621 | 0.9185 |
| m3 = −1.221*THBS4 + 1.994*IL6R + 1.837*AKT2 | 0.8591 | 0.9301 |
| m4 = 1.355*ETV7 + 1.944*TBL1X − 2.368*PPP2CB | 0.8468 | 0.9412 |
| m5 = 1.241*IRF7 + 2.258*TIFA + 0.887*CA9 | 0.8664 | 0.9528 |
| m6 = 3.990*IRF2 − 1.151*CCL14 + 0.872*DMD | 0.8254 | 0.9547 |
| m7 = 1.758*HLA_B + 4.221*DNAJC14 − 2.810*CRY1 | 0.8125 | 0.9479 |
| m8 = 1.933*CD274 − 1.752*CCL17 + 1.740*BLM | 0.8505 | 0.9479 |
| m9 = 1.355*GNLY − 2.171*LFNG + 2.451*ACSL4 | 0.8397 | 0.9442 |
| m10 = −1.448*BOK − 1.496*SERPINF1 + 1.864*HERPUD1 | 0.8542 | 0.9534 |
| m11 = −2.248*RUNX1 + 2.132*PML + 1.041*RAB6B | 0.8640 | 0.9534 |
| m12 = 1.446*LAG3 − 1.814*CLCF1 + 0.919*SPINK1 | 0.8395 | 0.9565 |
| m13 = 1.000*MX1 − 2.077*GSR + 2.438*KDM6A | 0.7839 | 0.9553 |
| m14 = 1.098*STAT1 + 2.418*TERF1 + 1.782*PSIP1 | 0.8565 | 0.9553 |
| m15 = 2.049*DHX58 − 1.426*SNCA + 0.762*KCNK5 | 0.8395 | 0.9528 |
| m16 = 2.410*JAK2 + 1.809*PLK4 − 2.686*BCL10 | 0.8297 | 0.9534 |
| m17 = 2.197*CCL7 − 1.293*TNXB + 2.436*SMC1A | 0.8415 | 0.9522 |
| m18 = 1.591*HLA_A − 1.424*STK39 + 0.843*IL12A | 0.8385 | 0.9486 |
| m19 = 3.058*CD83 − 0.931*TBL1Y − 1.712*PIM3 | 0.8000 | 0.9537 |
| m20 = −0.769*ITGA2 + 1.698*TLR3 + 1.687*GMPS | 0.8156 | 0.9483 |
| m21 = 0.717*CXCL10 + 0.754*PRAME + 1.929*ARID1A | 0.8358 | 0.9510 |
| m22 = −1.249*TIMP3 − 2.431*ATP5F1 − 1.751*PLCG1 | 0.8187 | 0.9510 |
| m23 = 1.171*PDCD1LG2 + 1.834*SMC4 + 0.795*MAPK10 | 0.8186 | 0.9483 |
| m24 = −1.666*DNAJB2 + 2.735*MSL2 − 1.067*IRS1 | 0.8107 | 0.9442 |
| m25 = 1.465*TAP2 + 2.820*SOCS4 + 2.015*CBX3 | 0.8174 | 0.9469 |
| m26 = 0.549*GBP1 + 2.100*E2F3 − 0.346*COL9A3 | 0.8046 | 0.9456 |
| m27 = 1.224*DDX58 − 2.764*DNAJC10 + 1.582*UBXN2A | 0.8180 | 0.9483 |
| m28 = −1.436*COL1A1 + 3.344*PRDM1 − 1.495*BATF | 0.8560 | 0.9510 |
| m29 = 1.599*NFKB1 − 1.549*PTGR1 + 1.263*CD47 | 0.7880 | 0.9537 |
| m30 = −1.525*P4HB − 1.302*NTHL1 − 0.761*LIF | 0.7990 | 0.9524 |
| m31 = −1.946*VGLL4 − 1.274*PCOLCE + 2.150*DNAJC8 | 0.8199 | 0.9510 |
| m32 = 1.059*CD79A − 1.337*TMEM74B + 1.437*PRC1 | 0.8425 | 0.9510 |
| m33 = 0.604*SLAMF7 − 1.613*GSN − 1.661*NAMPT | 0.8309 | 0.9524 |
| m34 = 0.709*IFI27 − 0.976*COL1A2 − 1.000*FASN | 0.8121 | 0.9524 |

The AUC in the table above does not consider the covariables grading and tumor size. If they are added to a committee, its predictive performance is further improved. Examples:

TABLE 20

| Algorithm | AUC |
|---|---|
| m1 + m2 + m3 | 0.9301 |
| 0.486*(m1 + m2 + m3) + | 0.9412 |
| 2.44*G − 0.73*T | |
| m1 + m2 + m3 + m4 + m5 | 0.9528 |
| 0.446*(m1 + m2 + m3 + m4 + | 0.9608 |
| m5) + 3.16*G − 1.05*T | |

Here, G codes the pathological grading of the tumor at baseline where G=2 for grade 1 or grade 2 and G=3 for grade 3. T codes the tumor size at baseline with T=1 for cT1, T=2 for cT2, T=3 for cT3 and T=4 for cT4.

### Example 7

Same as Example 5, but with four (instead of two) genes per member, and covariables window, grading and tumor size (instead of no covariables) when determining the logistic regression coefficients for each member.

TABLE 21

| | AUC(mem- | |
|---|---|---|
| member | ber) | AUC(cum.) |
| m1 = 3.990*PSIP1 + 5.631*SOCS4 + 3.937*HERPUD1 − 2.888*PAG1 | 0.9348 | 0.9348 |
| m2 = 1.797*HLA_B − 1.881*THBS4 + 1.168*DMD + 1.273*MLLT3 | 0.8911 | 0.9593 |
| m3 = 2.438*TAP1 − 1.504*BATF + 5.611*MSL2 − 3.233*SRF | 0.8882 | 0.9743 |
| m4 = 2.134*HLA_A + 2.763*TBL1X + 1.568*MAPK10 − 3.343*MED12 | 0.8822 | 0.9886 |
| m5 = 1.312*STAT1 + 1.059*CA9 + 2.464*TIFA − 1.863*LRP12 | 0.8624 | 0.9839 |
| m6 = −1.629*IRS1 − 1.210*RAC3 − 2.458*RB1 − 1.464*TNFRSF11B | 0.8953 | 0.9934 |
| m7 = 1.463*GBP1 + 2.807*PLK4 − 2.407*NOTCH1 − 2.175*PRMT6 | 0.8337 | 0.9892 |
| m8 = −2.263*BOK − 1.775*SLIT2 + 2.891*TLR3 − 1.659*TNFSF14 | 0.8822 | 0.9898 |
| m9 = −0.739*HEY2 − 3.252*CHMP4B − 1.163*BMP5 + 1.037*ETV7 | 0.8594 | 0.9880 |
| m10 = 2.856*IRF9 − 1.445*HIC1 + 1.792*IL12A − 1.591*CLCF1 | 0.8973 | 0.9851 |
| m11 = 3.711*JAK2 − 0.873*RELN − 5.264*BCL10 + 3.051*GMPS | 0.8720 | 0.9845 |
| m12 = 0.560*CXCL10 − 2.107*GSN + 3.398*KDM6A − 1.757*GSR | 0.8298 | 0.9813 |
| m13 = −1.021*ITGA2 − 0.769*CCL14 + 3.154*IRF2 + 0.747*RBP1 | 0.8541 | 0.9826 |
| m14 = 1.242*TAP2 + 3.056*IDH2 − 1.754*FASN − 4.031*KIF3B | 0.8475 | 0.9826 |
| m15 = 6.053*NFKB1 − 1.113*TBL1Y − 2.657*CXCL8 + 1.373*UGT1A1 | 0.8550 | 0.9785 |
| m16 = −1.795*PYCR1 − 1.933*DUSP6 + 2.354*RAD9A − 1.347*NTHL1 | 0.8517 | 0.9785 |
| m17 = −1.822*ID1 − 1.915*GNG12 + 2.344*MME − 1.669*PLCB1 | 0.8035 | 0.9772 |
| m18 = −1.827*TIMP3 − 3.178*BID − 3.132*STK3 − 2.893*JAK1 | 0.8224 | 0.9758 |
| m19 = −1.102*NOTCH4 + 1.588*CD38 − 2.288*CMKLR1 + 0.482*GSTM1 | 0.8786 | 0.9812 |
| m20 = 1.086*MX1 + 2.711*PARP2 − 0.671*CCL21 + 1.772*APAF1 | 0.8218 | 0.9852 |
| m21 = 1.879*LAG3 − 2.453*TNXB + 3.004*RAB6B − 1.512*NRG1 | 0.8690 | 0.9812 |
| m22 = 1.275*DNAJA1 − 1.483*ACSL3 − 1.853*NUMBL − 0.871*CCL17 | 0.8200 | 0.9785 |
| m23 = 1.645*IRF7 + 2.093*SMC4 + 2.288*DNAJC13 − 1.077*NR6A1 | 0.8050 | 0.9772 |
| m24 = 1.205*IFI27 + 2.270*MCM5 − 1.946*CCND3 + 3.238*DNAJC14 | 0.8278 | 0.9745 |
| m25 = −1.018*SORT1 − 0.650*SPDEF − 1.510*FOSL1 − 2.266*ARNT | 0.8110 | 0.9758 |

### Example 8

Committees can also be used to predict the benefit of durvalumab compared to placebo. The method is similar to the one described in Example 5, but in this example members are created from logistic regression models with interaction terms representing the interaction of the genes levels with the treatment arm, and the member coefficients (see table below) are taken from these interaction terms. Column “member” describes the mathematical definition of the members combining four genes each. A high score, e.g. over a certain threshold or cut-off, favors the durvalumab treatment for the respective patient, while a low score, e.g. below a certain threshold or cut-off, favors the placebo arm. Column “dAUC(member)” demonstrates the predictive performance measured as the AUC of the ROC in the durvalumab arm minus the AUC of the ROC in the placebo arm. Column “dAUC(cum.)” uses the same measure, but for the cumulated score similar to the table in Example 5. The pCR definition used here is ypT0/ypN0.

TABLE 22

| | dAUC(mem- | |
|---|---|---|
| member | ber) | dAUC(cum.) |
| m1 = −1.388*ADAMTS1 − 3.084*PIK3CA + 2.758*QSOX2 − 3.398*MED12 | 0.5082 | 0.5082 |
| m2 = −1.396*RUNX1 − 2.453*BID − 2.034*RAD51C + 1.536*PSIP1 | 0.3937 | 0.5645 |
| m3 = −1.038*HEY2 + 1.187*CHI3L1 − 0.894*LCN2 + 1.095*ER_154 | 0.5280 | 0.6006 |
| m4 = 2.780*IRF2 − 1.745*NOD2 + 0.911*ALDOC − 1.441*KDR | 0.4272 | 0.6196 |
| m5 = −2.078*TMEM74B + 1.978*TLR3 − 1.895*SELE + 1.199*GRIN2A | 0.4647 | 0.6566 |
| m6 = 0.799*HLA_A − 2.790*ALKBH3 − 2.180*NUMBL + 1.104*HSPA1L | 0.4617 | 0.6501 |
| m7 = −1.974*GSN + 1.617*HLA_B + 1.749*ERBB2 + 1.368*WWOX | 0.4280 | 0.6778 |
| m8 = −1.247*CCL28 + 1.401*AGT + 2.266*ID2 + 1.326*DDX58 | 0.4871 | 0.6979 |
| m9 = 2.358*DHX58 − 2.315*TNFRSF8 + 1.897*NTRK1 − 2.138*NLRP3 | 0.4765 | 0.7345 |
| m10 = 3.441*IDH1 − 1.708*FASN − 1.765*SERPINF1 − 2.769*ADIPOR1 | 0.4838 | 0.7405 |
| m11 = −1.749*HRK + 3.209*TERF1 − 1.202*NKD1 − 2.178*FAF1 | 0.4342 | 0.7720 |
| m12 = 3.124*MADD + 2.659*PPID − 2.712*TOP1 − 1.276*GADD45G | 0.4317 | 0.7583 |
| m13 = 3.582*MAX + 0.497*CA9 − 0.994*GPAT2 + 0.810*CCL25 | 0.3804 | 0.7648 |
| m14 = −2.049*CXCL8 + 2.146*GLIS3 − 1.736*LOXL1 + 2.543*CRK | 0.4254 | 0.7954 |
| m15 = −4.349*PTPN11 + 1.929*RPL13 + 1.879*PTP4A1 − 0.508*AREG | 0.4641 | 0.8050 |
| m16 = −1.268*CCL17 + 1.950*NAIP + 3.093*SOCS4 + 1.644*FANCG | 0.4254 | 0.7798 |
| m17 = −1.452*SLC45A3 + 3.087*TOP3A + 0.377*COL2A1 − 0.541*CCL18 | 0.4085 | 0.8039 |
| m18 = −1.957*CLCF1 − 2.502*COX7B + 2.386*FADD + 1.194*CXCL16 | 0.4274 | 0.8171 |
| m19 = 1.222*MLLT3 − 1.470*THBS4 − 1.431*CCNE2 + 2.050*DAAM1 | 0.3379 | 0.7911 |
| m20 = 1.997*TNFAIP3 − 0.569*ACKR2 − 0.739*CXCL1 − 1.002*PTPRC | 0.4155 | 0.8033 |
| m21 = −1.306*XRCC5 + 1.920*CYP4V2 − 2.038*CCT6B − 2.069*CCT4 | 0.4105 | 0.7962 |
| m22 = 0.707*NFKB1 − 1.075*DIABLO − 1.738*SPRY2 − 1.380*ZAK | 0.2771 | 0.8150 |
| m23 = −1.146*CEACAM3 − 1.416*KRT7 + 1.249*MESP1 + 2.338*SMAD2 | 0.4448 | 0.8158 |
| m24 = −0.807*PTCHD1 − 2.235*MAPK3 + 1.578*PFKFB3 + 2.584*EEF2K | 0.4373 | 0.8098 |
| m25 = −1.502*TMEM45B + 1.533*SCUBE2 + 1.194*ACSL5 + 2.118*NCOA2 | 0.4492 | 0.8147 |

If some cutoffs are applied to single members or committees, the pCR rates can be estimated in the respective subgroups:

TABLE 23

| | | pCR rate in | pCR rate in | pCR rate in | pCR rate in |
|---|---|---|---|---|---|
| | | durvalumab | durvalumab | placebo | placebo |
| | | arm if | arm if | arm if | arm if |
| algorithm | cutoff | expression high | expression low | expression high | expression low |
| m1 | −49.25 | 70% | 33% | 26% | 64% |
| m2 | −34.80 | 82% | 33% | 42% | 54% |
| m1 + m2 + m3 | −84.03 | 87% | 18% | 40% | 70% |
| m1 + . . . + m10 | −31.32 | 74% | 28% | 33% | 60% |

### Example 9

Same as Example 8 but with three (instead of four) genes per member and pCR defined as ypT0is/ypN0 (instead of ypT0/ypN0).

TABLE 24

| | dAUC(mem- | |
|---|---|---|
| member | ber) | dAUC(cum.) |
| m1 = −2.344*RUNX1 + 3.036*SPOP − 3.006*MED12 | 0.3920 | 0.3920 |
| m2 = 2.108*IL6R − 1.770*CCL17 + 2.404*AK3 | 0.3737 | 0.4526 |
| m3 = 2.686*DHX58 − 3.092*SERPINF1 + 1.163*VCAN | 0.4297 | 0.5219 |
| m4 = −1.470*COL1A1 − 2.476*ATP5F1 + 2.168*ACSL4 | 0.3602 | 0.5108 |
| m5 = −1.346*ADAMTS1 + 1.855*ITPKB + 1.143*HLA_A | 0.4358 | 0.5415 |
| m6 = 3.041*IRF2 + 1.112*MYBL1 + 1.725*PTP4A1 | 0.4333 | 0.5661 |
| m7 = 0.790*GNLY + 0.788*CHI3L1 + 0.955*RARB | 0.4190 | 0.5690 |
| m8 = −1.200*COL1A2 − 2.389*RAD51C + 2.601*SOCS4 | 0.4116 | 0.5783 |
| m9 = −1.514*PRKAA2 + 3.727*TERF1 − 1.888*SLC16A2 | 0.4714 | 0.6101 |
| m10 = 3.133*QSOX2 − 3.354*PIK3CA + 2.180*AKT2 | 0.3908 | 0.6385 |
| m11 = −2.026*COL5A1 + 1.663*GJA1 − 1.211*XRCC5 | 0.4076 | 0.6349 |
| m12 = 1.442*HLA_B + 1.379*PLA2G4A + 1.155*ACTR3B | 0.3974 | 0.6327 |
| m13 = −1.449*SFRP2 − 1.914*TK1 − 1.943*STK3 | 0.3445 | 0.6339 |
| m14 = −1.017*THBS4 + 0.725*CCL19 − 2.042*ALKBH3 | 0.3649 | 0.6318 |
| m15 = −2.398*MMP14 + 0.919*CA9 − 2.075*CCT4 | 0.3810 | 0.6203 |
| m16 = 2.003*EAF2 − 1.524*TMEM74B − 2.713*DNAJC10 | 0.3467 | 0.6233 |
| m17 = 1.483*BCL2A1 − 1.798*CLCF1 + 1.212*MESP1 | 0.3782 | 0.6233 |
| m18 = 2.222*PPID − 2.879*TOP1 − 0.931*COL3A1 | 0.3856 | 0.6286 |
| m19 = 1.576*DDX58 − 2.936*PPP2CA + 1.741*TBL1X | 0.3495 | 0.6298 |
| m20 = 1.632*HDAC8 + 1.501*JAK2 − 1.227*STK39 | 0.3983 | 0.6333 |
| m21 = −0.963*HEY2 + 1.285*C5orf55 + 1.240*PLCG2 | 0.3947 | 0.6212 |
| m22 = 1.527*LAG3 − 1.433*WNT10A + 1.411*CELSR2 | 0.4304 | 0.6244 |
| m23 = −2.410*TADA3 + 2.193*TOP3A − 0.646*GPAT2 | 0.3967 | 0.6314 |
| m24 = 1.875*CD47 − 2.638*VEGFB + 1.243*HSPA1A | 0.3277 | 0.6248 |
| m25 = −1.220*TIMP3 − 2.392*PSMD2 − 1.767*MAP3K5 | 0.3540 | 0.6168 |
| m26 = −1.383*P4HB − 1.572*TMEM45B + 1.219*GPR17 | 0.3893 | 0.6299 |
| m27 = 2.139*TAP2 + 3.714*DNAJC8 − 2.549*NOD2 | 0.3695 | 0.6196 |
| m28 = 3.479*MAT2A + 1.079*CCL7 − 2.281*FBXW11 | 0.3501 | 0.6248 |
| m29 = −2.504*NSD1 − 0.431*LCN2 + 1.514*NCOA2 | 0.3726 | 0.6341 |
| m30 = −1.357*GSN + 1.262*ITGB7 − 0.928*AR | 0.3350 | 0.6168 |
| m31 = 1.846*RASSF1 − 1.151*FASN + 2.588*EEF2K | 0.3874 | 0.6269 |
| m32 = 1.733*CD38 − 2.887*RIPK3 − 2.360*DIABLO | 0.3297 | 0.6197 |
| m33 = 1.813*PSIP1 − 0.681*NMU + 1.953*SETD2 | 0.4093 | 0.6473 |
| m34 = −0.882*KRT7 − 0.500*NKD1 − 0.682*TBL1Y | 0.4010 | 0.6354 |

### Example 10

Same as Example 8 but with two (instead of four) genes per member and covariable window (instead of no covariables) in the logistic regression models.

TABLE 25

| | dAUC(mem- | |
|---|---|---|
| member | ber) | dAUC(cum.) |
| m1 = −1.480*ADAMTS1 − 2.294*PIK3CA | 0.3346 | 0.3346 |
| m2 = −3.510*MED12 − 1.495*GSN | 0.3174 | 0.4655 |
| m3 = −0.729*HEY2 − 1.796*RAD51C | 0.3005 | 0.4899 |
| m4 = 2.478*IRF2 − 0.980*CCL17 | 0.3274 | 0.5078 |
| m5 = −1.598*RUNX1 − 2.097*BID | 0.2297 | 0.4983 |
| m6 = 1.181*HLA_A − 1.348*NOD2 | 0.3636 | 0.5208 |
| m7 = −1.926*TMEM74B + 0.717*ORM2 | 0.3784 | 0.5411 |
| m8 = −0.894*CCL28 + 0.753*AGT | 0.2721 | 0.5939 |
| m9 = 1.870*IDH1 − 1.223*TSPAN13 | 0.2774 | 0.5938 |
| m10 = 2.298*PPID − 2.267*TOP1 | 0.3053 | 0.6178 |
| m11 = 1.697*DHX58 − 1.292*TNFRSF8 | 0.3497 | 0.6142 |
| m12 = 0.997*HLA_B + 0.736*CHI3L1 | 0.2695 | 0.5922 |
| m13 = −1.445*HRK + 2.083*TERF1 | 0.2522 | 0.5966 |
| m14 = 1.133*CEBPB − 1.934*ATP5F1 | 0.2459 | 0.6080 |
| m15 = 2.159*TLR3 − 2.189*NLRP3 | 0.3955 | 0.5996 |
| m16 = −0.782*NKD1 − 0.367*LCN2 | 0.2930 | 0.6101 |
| m17 = 2.831*MADD − 1.142*SELE | 0.2573 | 0.6030 |
| m18 = −0.935*GPAT2 + 0.730*CCL25 | 0.3235 | 0.6215 |
| m19 = −1.131*CLCF1 − 1.386*CCT4 | 0.3065 | 0.6216 |
| m20 = −1.015*CXCL8 + 1.466*PFKFB3 | 0.3163 | 0.6260 |
| m21 = −2.051*ALKBH3 − 1.548*NUMBL | 0.3701 | 0.6321 |
| m22 = 1.905*PSIP1 + 2.476*SOCS4 | 0.2684 | 0.6331 |
| m23 = 1.197*SLC16A1 − 1.163*FOSL1 | 0.3101 | 0.6331 |
| m24 = 2.810*MAX + 1.310*ERBB2 | 0.2499 | 0.6342 |
| m25 = 1.480*TNFAIP3 − 0.879*CCL22 | 0.2914 | 0.6373 |
| m26 = −1.304*SLC45A3 + 2.715*TOP3A | 0.3445 | 0.6243 |
| m27 = 1.925*NFKB1 + 0.815*ALDOC | 0.2388 | 0.6310 |
| m28 = −2.808*PTPN11 + 1.574*RPL13 | 0.3041 | 0.6351 |
| m29 = 1.078*MLLT3 − 0.832*THBS4 | 0.2407 | 0.6186 |
| m30 = −1.296*CEACAM3 + 1.325*CCL3 | 0.2907 | 0.6132 |
| m31 = −1.240*XRCC5 + 1.977*QSOX2 | 0.2997 | 0.6262 |
| m32 = −1.975*CRLF2 + 1.756*IFNA5 | 0.3422 | 0.6221 |
| m33 = −1.312*KDR + 0.808*ACSL5 | 0.2212 | 0.6176 |
| m34 = −0.995*PRKAA2 + 1.038*CYP4V2 | 0.2831 | 0.6206 |
| m35 = 1.907*UBB − 2.253*PRKAG1 | 0.3315 | 0.6188 |
| m36 = −0.747*DIABLO − 1.122*SPRY2 | 0.1665 | 0.6120 |
| m37 = −1.373*TMEM45B + 1.155*IFNW1 | 0.2839 | 0.6185 |
| m38 = −1.430*TADA3 − 0.642*SERPINB2 | 0.2442 | 0.6231 |
| m39 = 1.332*USF2 − 1.013*WWC1 | 0.2529 | 0.6124 |
| m40 = −1.872*MAPK3 + 1.785*CRK | 0.2648 | 0.6156 |
| m41 = −0.675*PTCHD1 + 1.117*FANCG | 0.2655 | 0.6065 |
| m42 = 0.819*CD47 + 1.769*MAP3K4 | 0.2348 | 0.6143 |
| m43 = 1.889*MAT2A − 1.768*PHB | 0.2822 | 0.6206 |
| m44 = 0.900*RARB − 0.573*PROM1 | 0.2611 | 0.6296 |
| m45 = −1.189*TNXB + 1.036*CCL7 | 0.2567 | 0.6229 |
| m46 = −0.942*PTTG1 + 0.608*CA9 | 0.2706 | 0.6235 |
| m47 = −1.333*HMGB3 − 1.132*SERPINF1 | 0.2510 | 0.6229 |
| m48 = 0.278*PAX6 − 0.555*CCL18 | 0.2611 | 0.6145 |
| m49 = −1.141*CDX2 + 1.070*MIXL1 | 0.2403 | 0.6111 |
| m50 = −1.296*STX1A − 1.410*PSMD2 | 0.2420 | 0.6176 |

Columns “dAUC(member)” and “dAUC(cum.)” in the table above do not consider the covariable window. If it is added to a committee, its predictive performance is further improved. Examples:

TABLE 26

| Algorithm | dAUC |
|---|---|
| m1 + m2 | 0.4655 |
| 0.838*(m1 + m2) + 0.915*W | 0.4838 |
| m1 + m4 + m6 | 0.4563 |
| 0.563*(m1 + m4 + m6) + 0.313*W | 0.4571 |

Here, W codes the window participation of the patient where W=0 (window=no) codes that the durvalumab/placebo treatment started at the same time as the chemo therapy, and W=1 (window=yes) codes that the durvalumab/placebo treatment started two weeks prior to the chemo therapy.

### Example 11

Same as Example 8 but with two (instead of four) genes per member and covariables grading and tumor size (instead of no covariables) in the logistic regression models.

TABLE 27

| | dAUC(mem- | |
|---|---|---|
| member | ber) | dAUC(cum.) |
| m1 = −1.389*ADAMTS1 − 2.238*PIK3CA | 0.3378 | 0.3378 |
| m2 = −2.822*PTPN11 − 1.573*GSN | 0.2767 | 0.4078 |
| m3 = −1.007*HEY2 − 1.993*EIF6 | 0.2664 | 0.4266 |
| m4 = 1.215*HLA_A − 3.036*MED12 | 0.3305 | 0.4498 |
| m5 = 1.247*HLA_B + 1.485*LRIG1 | 0.2429 | 0.4397 |
| m6 = 3.354*MADD − 0.976*TNXB | 0.2577 | 0.4431 |
| m7 = −1.428*TMEM74B + 1.686*TLR3 | 0.3472 | 0.4703 |
| m8 = 2.554*NFKB1 − 1.199*SELE | 0.2774 | 0.4818 |
| m9 = −1.437*RUNX1 − 2.103*BID | 0.2328 | 0.4870 |
| m10 = 2.511*IRF2 − 1.058*CCL17 | 0.3344 | 0.4906 |
| m11 = 3.507*MAX + 0.618*CA9 | 0.2831 | 0.5021 |
| m12 = −1.305*SLC45A3 + 2.383*TOP3A | 0.3359 | 0.5017 |
| m13 = −0.898*DIABLO − 1.205*SPRY2 | 0.1676 | 0.4973 |
| m14 = −2.438*CAD − 0.907*COL1A1 | 0.2026 | 0.4904 |
| m15 = −1.035*XRCC5 − 0.808*FGFR3 | 0.2731 | 0.4840 |
| m16 = −1.114*CXCL8 + 1.151*BCL2A1 | 0.3068 | 0.4928 |
| m17 = −1.849*TADA3 − 0.589*GPAT2 | 0.2879 | 0.4946 |
| m18 = −2.091*ATP6V0C + 1.817*IDH1 | 0.3185 | 0.5013 |
| m19 = 1.633*DHX58 − 1.288*TNFRSF8 | 0.3411 | 0.5013 |
| m20 = 1.843*TNFAIP3 − 1.305*TNFRSF9 | 0.2760 | 0.5065 |
| m21 = −1.599*WWC1 − 1.719*NUMBL | 0.2602 | 0.5038 |
| m22 = −1.050*HRK − 0.826*KRT7 | 0.2598 | 0.5024 |
| m23 = −0.585*CCL28 − 1.847*RAD51C | 0.3123 | 0.5151 |
| m24 = −0.483*NKD1 + 1.226*TAP1 | 0.2332 | 0.5222 |
| m25 = −0.964*ANGPT1 − 0.367*LCN2 | 0.2433 | 0.5196 |
| m26 = 1.688*PSIP1 − 1.541*CCT4 | 0.2696 | 0.5259 |
| m27 = −2.499*ATP6V1G2 + 1.895*CCDC103 | 0.3057 | 0.5412 |
| m28 = −1.423*MAPK3 − 1.429*HMGB3 | 0.2512 | 0.5381 |
| m29 = −1.152*CEACAM3 + 1.506*SLC11A1 | 0.2658 | 0.5428 |
| m30 = −0.802*MYCN − 1.178*P4HB | 0.2968 | 0.5440 |
| m31 = −1.664*ALKBH3 − 0.899*EPCAM | 0.2704 | 0.5438 |
| m32 = −1.002*PRKAA2 − 0.607*PROM1 | 0.2351 | 0.5466 |
| m33 = −0.462*FABP4 + 0.933*MLLT3 | 0.2392 | 0.5518 |
| m34 = 2.417*JAK2 − 1.341*CCR4 | 0.2589 | 0.5463 |
| m35 = −1.214*FOSL1 + 1.284*TAP2 | 0.2225 | 0.5374 |
| m36 = −1.141*TMEM45B + 1.042*SCUBE2 | 0.2482 | 0.5483 |
| m37 = −1.283*KRT18 − 0.749*THBS4 | 0.2245 | 0.5436 |
| m38 = −1.230*GPAM − 1.265*STX1A | 0.2760 | 0.5387 |
| m39 = 2.288*MAT2A − 2.305*TOP1 | 0.2902 | 0.5455 |
| m40 = −2.076*RPL6 + 2.402*MGEA5 | 0.2968 | 0.5354 |
| m41 = −0.800*LIF − 1.016*PYCR1 | 0.2340 | 0.5337 |
| m42 = −0.830*FGF13 + 2.179*MSL2 | 0.2160 | 0.5309 |
| m43 = −1.357*PLA2G10 + 1.105*BIRC7 | 0.2102 | 0.5240 |
| m44 = 1.040*GNLY − 1.054*FLT3 | 0.2546 | 0.5154 |
| m45 = −1.467*IFNAR1 + 0.589*ORM2 | 0.2514 | 0.5186 |
| m46 = 1.229*ACSL5 − 1.074*PTPRC | 0.3323 | 0.5236 |
| m47 = −1.444*CDX2 + 1.301*IFNA5 | 0.3355 | 0.5198 |
| m48 = −0.787*MYOD1 + 1.872*FAS | 0.2758 | 0.5289 |
| m49 = −1.052*CLCF1 + 0.849*LAG3 | 0.2546 | 0.5217 |
| m50 = 0.797*CHI3L1 + 2.022*MAP3K4 | 0.2415 | 0.5362 |

### Example 12

Same as Example 8 but with two (instead of four) genes per member and covariables grading, tumor size and window (instead of no covariables) in the logistic regression models.

TABLE 28

| | dAUC(mem- | |
|---|---|---|
| member | ber) | dAUC(cum.) |
| m1 = −1.395*ADAMTS1 − 2.401*PIK3CA | 0.3359 | 0.3359 |
| m2 = −2.845*PTPN11 − 1.609*GSN | 0.2773 | 0.4085 |
| m3 = −0.715*HEY2 − 2.890*MED12 | 0.3088 | 0.4606 |
| m4 = 1.399*HLA_A + 1.454*LRIG1 | 0.2706 | 0.4621 |
| m5 = 1.036*HLA_B + 0.712*CHI3L1 | 0.2774 | 0.4709 |
| m6 = 2.724*NFKB1 − 1.273*SELE | 0.2774 | 0.4894 |
| m7 = 3.320*MADD − 0.984*TNXB | 0.2601 | 0.4731 |
| m8 = −1.429*RUNX1 − 2.089*BID | 0.2322 | 0.4592 |
| m9 = −1.420*TMEM74B + 1.681*TLR3 | 0.3479 | 0.4794 |
| m10 = 2.531*IRF2 − 1.071*CCL17 | 0.3344 | 0.4949 |
| m11 = −2.451*CAD − 0.902*COL1A1 | 0.2009 | 0.4829 |
| m12 = −1.327*SLC45A3 + 2.428*TOP3A | 0.3359 | 0.4852 |
| m13 = −0.895*DIABLO − 1.238*SPRY2 | 0.1680 | 0.4761 |
| m14 = −0.877*FGFR3 − 2.086*TOP1 | 0.2863 | 0.4809 |
| m15 = 3.469*MAX + 0.621*CA9 | 0.2825 | 0.4978 |
| m16 = −1.101*CXCL8 + 1.139*BCL2A1 | 0.3067 | 0.4923 |
| m17 = −1.088*XRCC5 − 0.818*KRT7 | 0.2139 | 0.4941 |
| m18 = −0.613*CCL28 − 1.966*RAD51C | 0.3123 | 0.5220 |
| m19 = −1.824*TADA3 − 0.576*GPAT2 | 0.2867 | 0.5224 |
| m20 = 1.266*TNFAIP3 − 1.052*TNFRSF8 | 0.2744 | 0.5201 |
| m21 = 1.890*IDH1 − 2.081*ATP6V0C | 0.3172 | 0.5331 |
| m22 = −0.983*PRKAA2 − 0.337*LCN2 | 0.2856 | 0.5310 |
| m23 = −1.593*WWC1 − 1.798*NUMBL | 0.2534 | 0.5282 |
| m24 = 2.136*DHX58 − 1.514*CCR4 | 0.3300 | 0.5332 |
| m25 = −0.941*HRK − 0.607*PROM1 | 0.2580 | 0.5209 |
| m26 = −2.499*ATP6V1G2 + 1.878*CCDC103 | 0.3089 | 0.5491 |
| m27 = −1.192*CEACAM3 + 1.561*SLC11A1 | 0.2657 | 0.5562 |
| m28 = −1.374*HMGB3 − 0.450*FABP4 | 0.2139 | 0.5520 |
| m29 = −1.000*ANGPT1 − 1.571*RPL6 | 0.2559 | 0.5403 |
| m30 = −0.810*MYCN − 1.193*P4HB | 0.2962 | 0.5473 |
| m31 = −1.874*MAPK3 − 1.566*ALKBH3 | 0.2925 | 0.5547 |
| m32 = −1.238*GPAM − 1.276*STX1A | 0.2760 | 0.5440 |
| m33 = 1.282*TAP2 − 1.211*FOSL1 | 0.2219 | 0.5363 |
| m34 = 1.143*MLLT3 − 0.908*THBS4 | 0.2410 | 0.5402 |
| m35 = −0.476*NKD1 + 1.220*TAP1 | 0.2346 | 0.5353 |
| m36 = −0.828*PPARGC1A − 1.217*CCT4 | 0.2011 | 0.5338 |
| m37 = −0.804*TMEM45B + 1.623*FAS | 0.2558 | 0.5416 |
| m38 = −1.682*KRT18 − 2.032*ARNT | 0.1990 | 0.5418 |
| m39 = 2.300*MAT2A − 2.155*PHB | 0.2822 | 0.5431 |
| m40 = −1.063*CLCF1 + 0.836*LAG3 | 0.2526 | 0.5440 |
| m41 = −0.883*PLA2G10 + 0.602*ORM2 | 0.2941 | 0.5372 |
| m42 = −1.490*CDX2 + 1.210*BIRC7 | 0.2724 | 0.5301 |
| m43 = 1.220*ACSL5 − 1.058*PTPRC | 0.3330 | 0.5349 |
| m44 = −0.788*LIF − 1.003*PYCR1 | 0.2334 | 0.5329 |
| m45 = −2.338*ATP5F1 − 1.208*DLC1 | 0.2552 | 0.5303 |
| m46 = −1.102*EPCAM − 1.100*LYVE1 | 0.2973 | 0.5253 |
| m47 = 1.365*PSIP1 − 1.978*VHL | 0.2498 | 0.5296 |
| m48 = −1.272*CRLF2 + 1.453*GBP7 | 0.2973 | 0.5267 |
| m49 = −1.461*IFNAR1 − 1.220*ZAK | 0.2346 | 0.5287 |
| m50 = 2.099*JAK2 − 0.928*FLT3 | 0.2435 | 0.5297 |

### Example 13

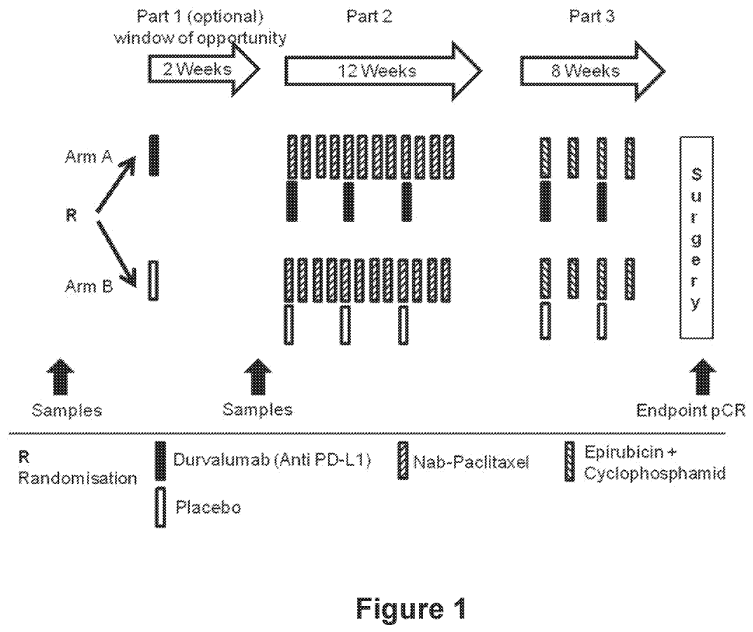

Some patients of the study participated in the window phase (see FIG. 1: part 1), and for some of them biopsy samples after this phase were analyzed. Three surprising observations were made for the dynamics of gene expressions (i.e. the difference between the log-normalized gene expression after window and the log-normalized gene expression before any treatment):

(i) For some genes the dynamic behavior differed significantly between the treatment arms.
(ii) For some genes the dynamic behavior predicted the pCR (ypT0/ypN0).
(iii) The sets (i) and (ii) of genes had a surprisingly high overlap (more than one would expect by the increase of pCR rates by durvalumab alone).

These observations allow the conclusion that genes showing a dynamic change under durvalumab treatment or different dynamic change when comparing durvalumab and placebo treated patients can be utilized to predict pCR and patient outcome.

The following table lists genes for which the dynamic expression (i.e. the gene expression after window minus the gene expression before window) is significantly different between arms and also significantly predicts pCR. Column “gene” shows the name of the gene. Column “pCR” contains “incr” if a dynamic increase of gene expression during the window phase is associated to a higher likelihood for a pCR (i.e. a dynamic decrease corresponds to a smaller likelihood of pCR); it contains “decr” if a dynamic decrease of gene expression during the window is associated to a higher likelihood of pCR (i.e. a dynamic increase corresponds to a smaller likelihood of pCR); column “p(pCR)” is the corresponding p-value from a t-test. Column “arm” contains “incr” if the dynamic increase of gene expression during the window phase is higher in the durvalumab arm compared to the placebo arm (i.e. the gene expression dynamically increases under durvalumab), it contains “decr” if the dynamic increase of gene expression is higher in the placebo arm compared to durvalumab (i.e. the gene expression dynamically decreases under durvalumab); column “p(arm)” is the corresponding p-value from a t-test.

TABLE 29

| gene | pCR | p(pCR) | arm | p(arm) |
|---|---|---|---|---|
| CASP4 | incr | 0.001003514 | incr | 0.040666506 |
| LRRK2 | incr | 0.001304999 | incr | 0.021727913 |
| GGH | decr | 0.002996595 | decr | 0.045801856 |
| C3AR1 | incr | 0.003453477 | incr | 0.018584697 |
| ARMC1 | decr | 0.003581366 | decr | 0.017324131 |
| FANCC | decr | 0.003756538 | decr | 0.049108662 |
| MAF | incr | 0.003835562 | incr | 0.011253993 |
| RASA1 | incr | 0.004562892 | incr | 0.000909671 |
| PIAS1 | incr | 0.005197408 | incr | 0.039203446 |
| HERC3 | incr | 0.006597379 | incr | 0.031873 |
| SLA | incr | 0.007288663 | incr | 0.048909772 |
| CFLAR | incr | 0.011559448 | incr | 0.027735362 |
| RUNX2 | incr | 0.012357206 | incr | 0.049546057 |
| FAF1 | decr | 0.016349683 | decr | 0.010270197 |
| CTLA4 | incr | 0.018093624 | incr | 0.037678338 |
| TNFSF14 | incr | 0.019373702 | incr | 0.026687842 |
| MAPKAPK5 | decr | 0.021763468 | decr | 0.040767992 |
| LAMA5 | decr | 0.022829245 | decr | 0.011753614 |
| PTEN | incr | 0.025222353 | incr | 0.015883766 |
| BID | incr | 0.028927858 | incr | 0.022722687 |
| FYN | incr | 0.030173569 | incr | 0.025563854 |
| E2F3 | decr | 0.033109865 | decr | 0.015185797 |
| ALDH1A1 | incr | 0.034432004 | incr | 0.006875953 |
| PDPN | incr | 0.03795828 | incr | 0.011005899 |
| NOX4 | incr | 0.042469606 | incr | 0.022995033 |
| MYBL2 | decr | 0.044578693 | decr | 0.037586345 |
| RBP1 | decr | 0.044663961 | decr | 0.030000495 |
| SYCP2 | decr | 0.048536113 | decr | 0.028816485 |

Surprisingly columns “pCR” and “arm” are identical. Looking at all genes analyzed, there is also a strong correlation between these two columns.

### Example 14 Gene Substitutions

The expression levels of some genes correlate highly; therefore a gene may be substituted by another one correlating to the first one. This may be useful in particular for multivariable score algorithms if some of the genes cannot be used to due legal or technical reasons. Substituting a gene will probably lead to an equivalent score in terms of prognosis or prediction for the endpoint or patient outcome. Gene substitution in the context of breast cancer biomarkers was previously described in patent application WO2013014296; the present invention uses the same mathematical methodology (unsupervised, based on z-transformations).

The following table lists genes from the examples above and points out potential substitutions. For most genes several alternative substitutions are available. Column “gene substitution” contains equations where the left side contains the gene to be substituted and the right side the mathematical expression for the substitution; the right side of the equation contains exactly one gene. Column “correlation” contains the Pearson correlation coefficient, which is a measure of the precision of the substitution.

TABLE 30

| gene substitution | correlation |
|---|---|
| ACKR2 = 1.48 * TTC9 − 1.67 | 0.474 |
| ACKR2 = 1.34 * CCL22 − 1.46 | 0.460 |
| ACKR2 = 1.28 * GPR160 − 1.46 | 0.453 |
| ACSL3 = 0.72 * FASN + 1.54 | 0.537 |
| ACSL3 = 1.20 * SLC19A2 − 0.43 | 0.500 |
| ACSL3 = −0.61 * GBP1 + 15.90 | −0.441 |
| ACSL4 = −0.70 * ZNF552 + 15.46 | −0.378 |
| ACSL4 = 0.83 * PAG1 + 2.95 | 0.376 |
| ACSL4 = −0.53 * FASN + 15.39 | −0.351 |
| ACSL5 = 1.11 * APOL3 − 2.70 | 0.684 |
| ACSL5 = 1.11 * CTSS − 4.00 | 0.661 |
| ACSL5 = 1.35 * TNFRSF1B − 4.97 | 0.652 |
| ACSL5 = 0.96 * BATF + 0.70 | 0.648 |
| ACSL5 = 0.88 * OAS1 + 0.19 | 0.625 |
| ACSL5 = 0.68 * CXCR3 + 2.86 | 0.617 |
| ACTA2 = 1.05 * TAGLN − 2.88 | 0.763 |
| ACTA2 = 1.63 * CALD1 − 7.27 | 0.670 |
| ACTA2 = 1.63 * PDLIM7 − 5.84 | 0.652 |
| ACTA2 = 1.27 * THBS2 − 4.22 | 0.646 |
| ACTA2 = 0.75 * EDIL3 + 4.02 | 0.605 |
| ACTA2 = 1.57 * TIMP2 − 8.60 | 0.594 |
| ACTR3B = −1.60 * DAB2 + 22.47 | −0.460 |
| ACTR3B = −1.21 * SLCO2B1 + 17.18 | −0.454 |
| ACTR3B = 1.99 * KMT2C − 14.34 | 0.445 |
| ADAMTS1 = 0.80 * PAK3 + 2.54 | 0.338 |
| ADAMTS1 = 1.13 * CDON + 0.63 | 0.331 |
| ADAMTS1 = 1.43 * TP53I3 − 3.06 | 0.322 |
| ADIPOR1 = −0.40 * PDCD1LG2 + 13.04 | −0.446 |
| ADIPOR1 = 1.09 * SP1 − 0.33 | 0.440 |
| ADIPOR1 = −0.28 * CD70 + 11.58 | −0.437 |
| AGT = 0.88 * CCL28 + 0.52 | 0.523 |
| AGT = 1.84 * PLCE1 − 6.62 | 0.510 |
| AGT = 0.95 * GATA5 + 2.30 | 0.508 |
| AHNAK = 0.85 * TIMP2 + 1.44 | 0.590 |
| AHNAK = 0.75 * LOXL1 + 4.68 | 0.569 |
| AHNAK = 0.92 * PDGFRB + 2.88 | 0.563 |
| AHNAK = 0.63 * COL5A2 + 4.53 | 0.558 |
| AHNAK = −1.15 * DNMT1 + 23.28 | −0.552 |
| AHNAK = −1.04 * CDC6 + 20.22 | −0.548 |
| AK3 = 0.64 * IFNA5 + 3.06 | 0.730 |
| AK3 = 0.58 * IFNW1 + 3.68 | 0.721 |
| AK3 = 0.67 * SLC22A9 + 2.80 | 0.718 |
| AK3 = 0.63 * IFNA2 + 3.38 | 0.717 |
| AK3 = 0.71 * IFNB1 + 2.37 | 0.710 |
| AK3 = 0.59 * MBL2 + 3.99 | 0.709 |
| AK3 = 0.54 * CCL1 + 4.85 | 0.702 |
| AKT2 = 0.61 * MAPKAPK2 + 5.17 | 0.642 |
| AKT2 = 1.05 * CAMKK2 + 1.08 | 0.553 |
| AKT2 = 1.02 * HMGXB3 + 1.81 | 0.536 |
| AKT2 = 1.16 * ACTR1B − 1.27 | 0.517 |
| AKT2 = 0.77 * ZNF589 + 3.68 | 0.504 |
| AKT2 = 0.95 * BTRC + 2.55 | 0.503 |
| ALDH1A3 = 1.10 * MACC1 − 1.59 | 0.462 |
| ALDH1A3 = 0.83 * PRR15L + 1.94 | 0.441 |
| ALDH1A3 = 0.98 * EMP1 − 2.21 | 0.437 |
| ALDOC = 0.81 * NDRG1 − 1.57 | 0.479 |
| ALDOC = 0.73 * ANGPTL4 + 1.67 | 0.449 |
| ALDOC = 1.03 * ADM − 2.12 | 0.415 |
| ALKBH3 = 0.46 * GFRA1 + 5.80 | 0.511 |
| ALKBH3 = 0.65 * DNAJC12 + 3.80 | 0.498 |
| ALKBH3 = 0.63 * ASB9 + 3.73 | 0.448 |
| ANGPT1 = 0.86 * RSPO2 + 2.55 | 0.615 |
| ANGPT1 = 0.90 * DNAJB7 + 2.08 | 0.562 |
| ANGPT1 = −1.51 * VAMP8 + 23.55 | −0.541 |
| ANGPT1 = 0.99 * ATP6V1G2 + 1.41 | 0.536 |
| ANGPT1 = 0.85 * DNAJC5B + 2.48 | 0.532 |
| ANGPT1 = 0.98 * IBSP + 1.04 | 0.530 |
| APAF1 = −1.10 * TOMM40 + 17.78 | −0.519 |
| APAF1 = 0.76 * BBS4 + 2.50 | 0.454 |
| APAF1 = 0.79 * RAMP2 + 0.62 | 0.439 |
| AR = 0.81 * TMEM45B + 2.08 | 0.810 |
| AR = 0.87 * HMGCS2 + 1.07 | 0.788 |
| AR = 0.85 * UGT1A6 + 1.83 | 0.762 |
| AR = 0.82 * ABCC12 + 1.97 | 0.751 |
| AR = 0.84 * UGT1A4 + 2.02 | 0.737 |
| AR = 0.80 * TAT + 2.22 | 0.725 |
| AR = 1.11 * ACVR1C − 0.64 | 0.725 |
| AR = 0.79 * UGT1A1 + 2.52 | 0.716 |
| AR = 0.83 * SERPINA9 + 2.17 | 0.712 |
| AR = 0.92 * S100A8 + 0.85 | 0.710 |
| AREG = 2.35 * ZAK − 12.13 | 0.372 |
| AREG = 1.62 * RAB27B − 5.86 | 0.371 |
| AREG = 1.74 * S100A6 − 18.98 | 0.367 |
| ARID1A = 0.49 * STMN1 + 4.54 | 0.438 |
| ARID1A = 0.80 * KDM1A + 2.37 | 0.423 |
| ARID1A = −0.38 * WNT7B + 12.82 | −0.417 |
| ARNT = −0.73 * KRT18 + 17.53 | −0.457 |
| ARNT = 1.11 * KDM5C − 2.29 | 0.441 |
| ARNT = −0.30 * IL3 + 10.39 | −0.424 |
| ATP5F1 = 0.83 * BCCIP + 2.38 | 0.444 |
| ATP5F1 = 1.02 * HMGB1 − 1.29 | 0.441 |
| ATP5F1 = −0.25 * ER_171 + 10.09 | −0.413 |
| ATP6V0C = 0.84 * VEGFB + 2.23 | 0.567 |
| ATP6V0C = 0.41 * SLC7A5 + 7.16 | 0.548 |
| ATP6V0C = 0.93 * STUB1 + 2.54 | 0.533 |
| ATP6V0C = 0.99 * SLC3A2 + 0.63 | 0.521 |
| ATP6V0C = 0.91 * TADA3 + 2.16 | 0.512 |
| ATP6V0C = 0.46 * STAB1 + 7.81 | 0.506 |
| ATP6V1G2 = 0.84 * APCS + 0.91 | 0.875 |
| ATP6V1G2 = 0.81 * ITLN2 + 1.36 | 0.875 |
| ATP6V1G2 = 0.76 * RXRG + 1.88 | 0.856 |
| ATP6V1G2 = 0.81 * IL17A + 2.11 | 0.853 |
| ATP6V1G2 = 0.80 * OR10J3 + 1.22 | 0.851 |
| ATP6V1G2 = 0.72 * SOX3 + 2.38 | 0.850 |
| ATP6V1G2 = 0.87 * EPOR + 1.37 | 0.849 |
| ATP6V1G2 = 0.77 * THPO + 1.89 | 0.847 |
| ATP6V1G2 = 0.78 * S100A8 + 1.39 | 0.847 |
| ATP6V1G2 = 1.05 * DPPA4 − 1.68 | 0.845 |
| BATF = 1.04 * IL2RB − 1.59 | 0.727 |
| BATF = 1.14 * CCR5 − 1.92 | 0.726 |
| BATF = 0.95 * CD2 − 1.08 | 0.726 |
| BATF = 0.76 * CD27 + 1.32 | 0.725 |
| BATF = 0.99 * PRF1 − 0.54 | 0.724 |
| BATF = 1.34 * CASP10 − 3.62 | 0.711 |
| BATF = 0.80 * GZMB + 0.57 | 0.708 |
| BATF = 0.62 * IRF4 + 2.54 | 0.707 |
| BATF = 1.33 * IRF1 − 2.91 | 0.702 |
| BCL10 = 0.79 * FAF1 + 2.07 | 0.457 |
| BCL10 = 0.91 * FUBP1 − 0.66 | 0.422 |
| BCL10 = 0.85 * GNAI3 + 0.35 | 0.391 |
| BCL2A1 = 0.76 * CCL5 + 1.17 | 0.608 |
| BCL2A1 = 0.91 * LAG3 + 1.53 | 0.589 |
| BCL2A1 = 0.76 * GNLY + 2.23 | 0.577 |
| BCL2A1 = 1.48 * CD86 − 3.89 | 0.572 |
| BCL2A1 = 0.94 * PRF1 + 1.23 | 0.569 |
| BCL2A1 = 1.08 * TNFAIP2 − 1.10 | 0.569 |
| BID = 0.69 * TLR6 + 2.75 | 0.390 |
| BID = 0.55 * NANOG + 3.47 | 0.381 |
| BID = 0.64 * MAP3K13 + 4.06 | 0.354 |
| BIRC7 = 0.88 * PTCHD2 + 0.63 | 0.794 |
| BIRC7 = 0.85 * GDF6 + 1.48 | 0.793 |
| BIRC7 = 0.98 * CSF2 + 0.30 | 0.784 |
| BIRC7 = 0.94 * GATA1 + 0.43 | 0.780 |
| BIRC7 = 1.01 * SOX3 − 0.15 | 0.779 |
| BIRC7 = 0.91 * ADRA1D + 1.19 | 0.778 |
| BIRC7 = 0.94 * HAND1 + 0.67 | 0.777 |
| BIRC7 = 0.86 * T + 0.87 | 0.772 |
| BIRC7 = 1.36 * CHEK1 − 2.73 | 0.771 |
| BIRC7 = 1.03 * SLC3A1 − 0.75 | 0.768 |
| BLM = 0.98 * FAM64A − 1.07 | 0.707 |
| BLM = 0.89 * CDK1 + 1.31 | 0.690 |
| BLM = 0.62 * SLC7A9 + 3.38 | 0.682 |
| BLM = 0.46 * DLL3 + 4.89 | 0.661 |
| BLM = 0.60 * DNAJC5G + 3.96 | 0.647 |
| BLM = 0.61 * APCS + 3.23 | 0.640 |
| BMP5 = 1.06 * SLC22A2 − 0.23 | 0.781 |
| BMP5 = 0.99 * IL17F − 0.02 | 0.780 |
| BMP5 = 1.05 * SLC22A9 − 0.18 | 0.759 |
| BMP5 = 0.99 * IL17A + 1.16 | 0.754 |
| BMP5 = 1.12 * DPPA2 − 3.09 | 0.751 |
| BMP5 = 1.08 * GSTA2 − 0.77 | 0.747 |
| BMP5 = 0.97 * NRG4 + 0.74 | 0.746 |
| BMP5 = 1.06 * CYP3A4 − 0.21 | 0.746 |
| BMP5 = 1.01 * CYP3A5 − 0.50 | 0.742 |
| BMP5 = 1.02 * CACNA1E + 0.13 | 0.741 |
| BOK = −0.66 * GZMA + 14.01 | −0.544 |
| BOK = −0.72 * IL2RG + 15.42 | −0.525 |
| BOK = −1.12 * CD86 + 18.47 | −0.506 |
| BOK = −0.52 * CXCL10 + 14.29 | −0.504 |
| BOK = −0.68 * CD3D + 14.76 | −0.501 |
| C5orf55 = 0.67 * AHRR + 2.76 | 0.611 |
| C5orf55 = −1.26 * HSPA4 + 20.48 | −0.535 |
| C5orf55 = −1.36 * DNAJA1 + 22.15 | −0.504 |
| CA9 = 1.10 * ANGPTL4 − 0.99 | 0.563 |
| CA9 = 1.55 * ADM − 6.69 | 0.555 |
| CA9 = 2.03 * BNIP3 − 13.88 | 0.512 |
| CAD = 0.92 * DNMT3A + 0.33 | 0.440 |
| CAD = 0.41 * MCM2 + 5.30 | 0.439 |
| CAD = 1.10 * MED24 − 0.86 | 0.422 |
| CASP8AP2 = 0.92 * NASP − 1.05 | 0.560 |
| CASP8AP2 = 0.82 * MCM5 + 0.22 | 0.529 |
| CASP8AP2 = 0.75 * FANCL + 2.01 | 0.517 |
| CAV1 = 1.08 * CAV2 − 0.53 | 0.727 |
| CAV1 = 1.09 * PDGFRB − 1.04 | 0.557 |
| CAV1 = 0.81 * FLRT2 + 2.78 | 0.517 |
| CAV2 = 0.92 * CAV1 + 0.49 | 0.727 |
| CAV2 = 1.00 * PDGFRB − 0.43 | 0.556 |
| CAV2 = 0.99 * CALD1 − 1.54 | 0.545 |
| CAV2 = 0.95 * PDGFA + 1.12 | 0.528 |
| CAV2 = 0.77 * MET + 2.55 | 0.515 |
| CAV2 = −0.63 * LAG3 + 13.64 | −0.510 |
| CBX3 = 0.86 * H3F3A − 0.04 | 0.510 |
| CBX3 = −0.56 * ACACB + 15.66 | −0.492 |
| CBX3 = 0.67 * RRM1 + 5.09 | 0.462 |
| CCDC103 = 0.96 * CCL3 − 0.13 | 0.805 |
| CCDC103 = 0.85 * THPO + 0.31 | 0.793 |
| CCDC103 = 0.94 * AURKC − 0.14 | 0.792 |
| CCDC103 = 0.88 * RPA3 + 0.51 | 0.788 |
| CCDC103 = 0.88 * ITLN2 − 0.26 | 0.782 |
| CCDC103 = 0.80 * DKK4 + 0.73 | 0.780 |
| CCDC103 = 0.83 * GLI1 + 0.49 | 0.779 |
| CCDC103 = 1.17 * ANG − 2.08 | 0.776 |
| CCDC103 = 0.68 * CACNG6 + 1.86 | 0.774 |
| CCDC103 = 0.71 * HNF1B + 1.57 | 0.774 |
| CCL14 = 1.12 * ACKR1 − 0.56 | 0.833 |
| CCL14 = 1.06 * TNXB − 0.52 | 0.763 |
| CCL14 = 1.35 * IGF1 − 3.79 | 0.754 |
| CCL14 = 1.44 * ABCA9 − 2.99 | 0.752 |
| CCL14 = 1.51 * TSPAN7 − 3.68 | 0.736 |
| CCL14 = 1.24 * IL33 − 2.56 | 0.729 |
| CCL14 = 1.58 * S1PR1 − 3.35 | 0.719 |
| CCL17 = 0.93 * IL12B + 0.06 | 0.728 |
| CCL17 = 1.06 * XCR1 − 1.11 | 0.724 |
| CCL17 = 1.27 * SNAI3 − 2.87 | 0.722 |
| CCL17 = 0.85 * SERPINA9 + 0.54 | 0.713 |
| CCL17 = 0.94 * LTA + 0.39 | 0.710 |
| CCL17 = 0.80 * MADCAM1 + 1.11 | 0.708 |
| CCL17 = 0.88 * NR0B2 + 1.47 | 0.707 |
| CCL17 = 0.93 * ESR2 + 0.42 | 0.704 |
| CCL17 = 1.57 * MFNG − 6.27 | 0.703 |
| CCL17 = 1.12 * MS4A1 − 2.00 | 0.702 |
| CCL18 = 1.29 * CCL13 − 0.99 | 0.629 |
| CCL18 = 1.36 * FBP1 − 1.98 | 0.559 |
| CCL18 = 2.07 * NR1H3 − 6.75 | 0.555 |
| CCL18 = 1.91 * IL2RA − 5.51 | 0.503 |
| CCL19 = 2.07 * TCF7 − 9.36 | 0.682 |
| CCL19 = 2.10 * PRKCB − 8.30 | 0.679 |
| CCL19 = 1.83 * CD52 − 7.80 | 0.675 |
| CCL19 = 1.66 * CCR7 − 1.89 | 0.651 |
| CCL19 = 2.09 * RASGRP2 − 7.99 | 0.650 |
| CCL19 = 1.49 * LTB − 3.53 | 0.649 |
| CCL21 = 1.70 * RASGRP2 − 5.39 | 0.662 |
| CCL21 = 1.33 * ACKR1 − 0.99 | 0.644 |
| CCL21 = 1.07 * FCER2 + 2.92 | 0.633 |
| CCL21 = 1.35 * CCR7 − 0.41 | 0.625 |
| CCL21 = 1.18 * CCL14 − 0.33 | 0.615 |
| CCL21 = 1.40 * CXCR5 − 1.47 | 0.613 |
| CCL22 = 1.49 * ENTPD1 − 3.92 | 0.687 |
| CCL22 = 1.07 * SNAI3 + 0.30 | 0.685 |
| CCL22 = 1.03 * CCR6 + 0.45 | 0.683 |
| CCL22 = 0.85 * CCL17 + 2.68 | 0.680 |
| CCL22 = 1.14 * CCR4 − 1.87 | 0.674 |
| CCL22 = 0.91 * CXCR5 + 0.82 | 0.664 |
| CCL25 = 0.92 * ER_099 + 0.89 | 0.771 |
| CCL25 = 0.76 * CCL27 + 1.05 | 0.762 |
| CCL25 = 1.02 * ER_120 + 1.23 | 0.752 |
| CCL25 = 0.86 * SLC22A6 + 0.87 | 0.748 |
| CCL25 = 0.85 * ER_067 + 1.01 | 0.736 |
| CCL25 = 0.76 * DNTT + 1.48 | 0.731 |
| CCL25 = 0.85 * ER_013 + 1.37 | 0.727 |
| CCL25 = 0.83 * ABCB11 + 0.93 | 0.726 |
| CCL25 = 0.88 * GML + 0.70 | 0.713 |
| CCL25 = 0.93 * UTY + 1.63 | 0.701 |
| CCL28 = 1.14 * AGT − 0.59 | 0.523 |
| CCL28 = 1.35 * PRR15L − 3.09 | 0.492 |
| CCL28 = 0.66 * LCN2 + 2.67 | 0.470 |
| CCL3 = 0.79 * SLC28A2 + 1.09 | 0.869 |
| CCL3 = 0.93 * DPPA5 − 0.85 | 0.866 |
| CCL3 = 0.89 * THPO + 0.46 | 0.860 |
| CCL3 = 0.80 * SSX1 − 0.42 | 0.858 |
| CCL3 = 0.85 * LMO2 + 0.88 | 0.857 |
| CCL3 = 0.81 * SERPINA9 + 1.18 | 0.857 |
| CCL3 = 0.99 * AURKC − 0.01 | 0.855 |
| CCL3 = 0.88 * AQP7 − 1.40 | 0.851 |
| CCL3 = 0.86 * IL12B + 0.87 | 0.849 |
| CCL3 = 0.88 * NPPB + 0.71 | 0.848 |
| CCL4 = 1.01 * C1QA − 3.19 | 0.749 |
| CCL4 = 0.68 * SLAMF7 + 2.21 | 0.742 |
| CCL4 = 0.81 * CCL5 + 0.18 | 0.729 |
| CCL4 = 1.22 * IL10RA − 3.37 | 0.721 |
| CCL4 = 1.20 * FGL2 − 4.33 | 0.718 |
| CCL4 = 1.04 * CYBB − 3.00 | 0.713 |
| CCL4 = 1.17 * CTSS − 4.16 | 0.703 |
| CCL5 = 1.29 * IL2RB − 1.22 | 0.862 |
| CCL5 = 1.23 * IL2RG − 1.35 | 0.858 |
| CCL5 = 1.20 * CD8A − 0.41 | 0.825 |
| CCL5 = 1.17 * CD3D − 0.22 | 0.825 |
| CCL5 = 1.47 * FGL2 − 5.55 | 0.822 |
| CCL5 = 1.43 * CTSS − 5.30 | 0.811 |
| CCL5 = 0.99 * GNLY + 1.39 | 0.809 |
| CCL5 = 1.18 * CD2 − 0.60 | 0.799 |
| CCL5 = 1.43 * APOL3 − 3.62 | 0.799 |
| CCL5 = 1.36 * STAT1 − 6.60 | 0.793 |
| CCL7 = 1.48 * AQP9 − 4.99 | 0.656 |
| CCL7 = 1.14 * CCR3 − 0.24 | 0.616 |
| CCL7 = 1.37 * SLC11A1 − 2.86 | 0.603 |
| CCL7 = 1.44 * GBP7 − 3.52 | 0.598 |
| CCL7 = 1.25 * CD274 − 2.63 | 0.598 |
| CCL7 = 1.06 * IFNA5 − 0.20 | 0.591 |
| CCND3 = 0.83 * CNPY3 + 2.99 | 0.463 |
| CCND3 = 1.04 * CREBBP − 0.44 | 0.428 |
| CCND3 = 1.10 * SRF − 0.85 | 0.420 |
| CCNE2 = 1.24 * PTTG2 − 3.03 | 0.527 |
| CCNE2 = 1.86 * HMGB1 − 11.35 | 0.519 |
| CCNE2 = 1.26 * ECT2 − 3.45 | 0.514 |
| CCNE2 = −1.43 * TGFBR2 + 23.13 | −0.508 |
| CCNE2 = 1.10 * HMGB2 − 2.79 | 0.506 |
| CCNE2 = 1.12 * GPSM2 − 1.77 | 0.506 |
| CCR4 = 0.85 * CD5 + 2.13 | 0.860 |
| CCR4 = 0.97 * PRKCB − 0.03 | 0.824 |
| CCR4 = 0.84 * CCR2 + 1.71 | 0.814 |
| CCR4 = 0.90 * CTLA4 + 1.05 | 0.799 |
| CCR4 = 1.10 * IL16 − 0.76 | 0.779 |
| CCR4 = 0.78 * CD2 + 1.09 | 0.779 |
| CCR4 = 0.77 * CCR7 + 2.95 | 0.778 |
| CCR4 = 0.98 * MAP4K1 − 0.06 | 0.776 |
| CCR4 = 0.90 * IRF8 − 0.72 | 0.773 |
| CCR4 = 1.03 * KLRG1 − 0.13 | 0.766 |
| CCT4 = 0.68 * ARAF + 4.22 | 0.780 |
| CCT4 = 0.72 * YY1 + 4.11 | 0.761 |
| CCT4 = 0.86 * ANAPC2 + 2.88 | 0.731 |
| CCT4 = 0.86 * CMC2 + 3.48 | 0.727 |
| CCT4 = 1.34 * MEN1 − 1.67 | 0.723 |
| CCT4 = 0.64 * MMS19 + 4.87 | 0.714 |
| CCT4 = 0.95 * FAM162A + 2.23 | 0.711 |
| CCT4 = 0.98 * H2AFX + 0.83 | 0.707 |
| CCT4 = 0.77 * ORC6 + 4.18 | 0.705 |
| CCT4 = 0.63 * DNAJC7 + 5.11 | 0.701 |
| CCT6B = 0.73 * F8 + 2.30 | 0.649 |
| CCT6B = 0.59 * TDGF1 + 3.17 | 0.648 |
| CCT6B = 0.54 * CYP2C9 + 4.41 | 0.646 |
| CCT6B = 0.55 * CYP3A5 + 3.46 | 0.645 |
| CCT6B = 0.54 * KLB + 4.10 | 0.643 |
| CCT6B = 0.59 * IL5 + 3.93 | 0.642 |
| CD274 = 1.27 * IRF1 − 2.36 | 0.781 |
| CD274 = 1.08 * CCR5 − 1.43 | 0.778 |
| CD274 = 1.05 * TBX21 + 0.22 | 0.757 |
| CD274 = 0.90 * LAG3 + 0.32 | 0.748 |
| CD274 = 1.16 * CD80 − 0.19 | 0.746 |
| CD274 = 1.25 * TNFRSF9 − 1.11 | 0.739 |
| CD274 = 0.90 * CD8A − 0.32 | 0.720 |
| CD274 = 0.97 * IL2RB − 0.94 | 0.715 |
| CD274 = 0.86 * GZMA + 0.76 | 0.714 |
| CD274 = 1.15 * FASLG − 0.56 | 0.713 |
| CD38 = 0.87 * SLAMF7 + 0.16 | 0.862 |
| CD38 = 1.20 * PIM2 − 3.33 | 0.843 |
| CD38 = 0.80 * IRF4 + 1.63 | 0.833 |
| CD38 = 1.56 * IL10RA − 6.95 | 0.826 |
| CD38 = 1.28 * IL2RG − 3.83 | 0.811 |
| CD38 = 0.98 * CD27 + 0.05 | 0.805 |
| CD38 = 0.98 * CD79A + 0.17 | 0.792 |
| CD38 = 1.34 * IL2RB − 3.69 | 0.791 |
| CD38 = 1.72 * IRF1 − 5.40 | 0.790 |
| CD38 = 1.46 * CCR5 − 4.12 | 0.789 |
| CD47 = 0.91 * IFT52 + 1.43 | 0.804 |
| CD47 = 0.84 * GADD45A + 1.70 | 0.755 |
| CD47 = 1.21 * CEBPB − 5.14 | 0.715 |
| CD47 = 2.21 * RIPK1 − 9.77 | 0.706 |
| CD47 = 1.75 * RHOA − 12.16 | 0.697 |
| CD47 = 1.84 * POLR2D − 7.95 | 0.681 |
| CD55 = 0.66 * THBS2 + 3.21 | 0.572 |
| CD55 = −0.56 * LAG3 + 14.79 | −0.561 |
| CD55 = −0.70 * SOCS1 + 17.07 | −0.557 |
| CD55 = −0.57 * PRF1 + 14.98 | −0.545 |
| CD55 = −0.60 * IL2RB + 15.58 | −0.543 |
| CD55 = 1.19 * ITGB1 − 4.24 | 0.542 |
| CD79A = 1.22 * PIM2 − 3.57 | 0.885 |
| CD79A = 1.17 * TNFRSF17 − 0.90 | 0.866 |
| CD79A = 0.82 * IRF4 + 1.49 | 0.851 |
| CD79A = 1.02 * CD38 − 0.17 | 0.792 |
| CD79A = 1.76 * CASP10 − 6.61 | 0.769 |
| CD79A = 1.00 * CD27 − 0.12 | 0.751 |
| CD79A = 1.61 * XBP1 − 11.93 | 0.746 |
| CD79A = 1.35 * CCR2 − 2.27 | 0.744 |
| CD79A = 2.26 * EAF2 − 9.78 | 0.744 |
| CD79A = 0.88 * SLAMF7 − 0.01 | 0.743 |
| CD83 = 0.72 * SELE + 3.55 | 0.427 |
| CD83 = −0.86 * BOK + 16.48 | −0.402 |
| CD83 = −0.92 * RASSF7 + 17.08 | −0.395 |
| CD86 = 1.05 * HAVCR2 − 0.47 | 0.882 |
| CD86 = 0.92 * SLC7A7 + 0.21 | 0.837 |
| CD86 = 0.74 * CTSS + 0.66 | 0.819 |
| CD86 = 0.76 * FGL2 + 0.55 | 0.797 |
| CD86 = 0.66 * CYBB + 1.40 | 0.794 |
| CD86 = 1.09 * CASP1 − 1.90 | 0.787 |
| CD86 = 0.64 * C1QA + 1.28 | 0.785 |
| CD86 = 0.64 * IL2RG + 2.72 | 0.785 |
| CD86 = 0.73 * CXCR6 + 2.55 | 0.785 |
| CD86 = 0.73 * CCR5 + 2.58 | 0.780 |
| CD8A = 0.98 * CD3D + 0.15 | 0.890 |
| CD8A = 0.99 * CD2 − 0.16 | 0.881 |
| CD8A = 1.08 * IL2RB − 0.68 | 0.876 |
| CD8A = 1.03 * IL2RG − 0.79 | 0.870 |
| CD8A = 1.08 * CD52 − 1.28 | 0.857 |
| CD8A = 1.23 * FGL2 − 4.29 | 0.839 |
| CD8A = 1.18 * CXCR6 − 1.06 | 0.832 |
| CD8A = 0.73 * CXCR3 + 3.29 | 0.831 |
| CD8A = 0.83 * CCL5 + 0.34 | 0.825 |
| CD8A = 1.14 * IRF8 − 2.41 | 0.825 |
| CDC7 = 0.89 * TTK + 0.76 | 0.586 |
| CDC7 = 0.88 * BRIP1 + 2.11 | 0.522 |
| CDC7 = 1.31 * MSH6 − 4.17 | 0.519 |
| CDKN2A = 1.49 * CDKN2B − 3.14 | 0.505 |
| CDKN2A = 2.86 * DNAJA1 − 24.38 | 0.462 |
| CDKN2A = 2.09 * TFDP1 − 13.78 | 0.449 |
| CDX2 = 0.94 * MADCAM1 + 0.57 | 0.863 |
| CDX2 = 1.04 * KLK3 − 0.69 | 0.857 |
| CDX2 = 1.02 * OLIG2 + 0.13 | 0.854 |
| CDX2 = 1.04 * SLC3A1 − 1.04 | 0.852 |
| CDX2 = 1.12 * LCN1 − 2.30 | 0.852 |
| CDX2 = 0.99 * CRYAA − 0.21 | 0.852 |
| CDX2 = 1.01 * WNT7A + 0.03 | 0.848 |
| CDX2 = 0.96 * GATA1 + 0.10 | 0.847 |
| CDX2 = 1.10 * THPO − 1.09 | 0.835 |
| CDX2 = 1.06 * LMO2 − 0.62 | 0.834 |
| CEACAM3 = 0.91 * MYOD1 + 1.04 | 0.853 |
| CEACAM3 = 0.98 * PLA2G3 + 0.28 | 0.852 |
| CEACAM3 = 0.96 * LEP + 0.47 | 0.850 |
| CEACAM3 = 1.09 * PLA2G10 − 2.31 | 0.845 |
| CEACAM3 = 0.86 * CAMK2B + 1.26 | 0.826 |
| CEACAM3 = 1.27 * TIE1 − 2.27 | 0.821 |
| CEACAM3 = 0.80 * UTF1 + 1.92 | 0.819 |
| CEACAM3 = 0.90 * WNT1 + 0.58 | 0.818 |
| CEACAM3 = 0.99 * CMTM2 + 0.62 | 0.815 |
| CEACAM3 = 1.53 * TNFRSF10C − 5.16 | 0.805 |
| CEBPB = 0.76 * IFT52 + 5.41 | 0.771 |
| CEBPB = 1.52 * POLR2D − 2.33 | 0.757 |
| CEBPB = 0.83 * CD47 + 4.25 | 0.715 |
| CEBPB = 1.37 * RHOA − 4.85 | 0.678 |
| CEBPB = 0.74 * GADD45A + 5.23 | 0.661 |
| CEBPB = 1.49 * FKBP8 − 3.70 | 0.660 |
| CELSR2 = 1.03 * PSRC1 − 1.20 | 0.595 |
| CELSR2 = 1.11 * PRKAR1B − 0.76 | 0.523 |
| CELSR2 = 1.08 * GPSM2 − 2.55 | 0.499 |
| CHI3L1 = 1.02 * CHI3L2 + 1.69 | 0.478 |
| CHI3L1 = −1.07 * MLPH + 19.02 | −0.401 |
| CHI3L1 = 2.57 * CKS1B − 17.74 | 0.399 |
| CHMP4B = 0.74 * VAMP8 + 2.85 | 0.571 |
| CHMP4B = −0.41 * LAMC3 + 13.02 | −0.550 |
| CHMP4B = −0.58 * TGFB1 + 14.48 | −0.547 |
| CHMP4B = −0.39 * CDH3 + 12.68 | −0.540 |
| CHMP4B = −0.37 * GLI1 + 12.64 | −0.538 |
| CHMP4B = −0.39 * CYP2C19 + 12.51 | −0.537 |
| CLCF1 = 0.59 * RPRM + 4.08 | 0.602 |
| CLCF1 = 1.38 * POLD4 − 2.38 | 0.571 |
| CLCF1 = 0.73 * NTN3 + 2.46 | 0.568 |
| CLCF1 = 0.64 * TNNI3 + 3.08 | 0.560 |
| CLCF1 = 0.69 * NPPB + 2.86 | 0.560 |
| CLCF1 = 0.64 * PGR + 3.89 | 0.559 |
| CMKLR1 = 0.82 * CXCR6 + 0.44 | 0.749 |
| CMKLR1 = 1.08 * PIK3R5 − 0.29 | 0.735 |
| CMKLR1 = 0.74 * CCR2 + 1.63 | 0.733 |
| CMKLR1 = 0.71 * PRF1 + 1.47 | 0.733 |
| CMKLR1 = 1.13 * SLA − 3.18 | 0.723 |
| CMKLR1 = 0.88 * IL10RA − 1.13 | 0.719 |
| COL1A1 = 1.05 * COL1A2 + 2.00 | 0.953 |
| COL1A1 = 1.02 * COL3A1 + 0.30 | 0.942 |
| COL1A1 = 1.16 * COL5A2 + 2.59 | 0.901 |
| COL1A1 = 1.30 * SPARC − 1.58 | 0.900 |
| COL1A1 = 1.20 * COL5A1 + 1.63 | 0.891 |
| COL1A1 = 1.16 * MMP2 + 0.99 | 0.833 |
| COL1A1 = 1.18 * LOX + 4.23 | 0.819 |
| COL1A1 = 0.90 * SFRP2 + 4.60 | 0.814 |
| COL1A1 = 1.06 * FN1 − 0.17 | 0.807 |
| COL1A1 = 1.23 * FBN1 + 2.45 | 0.800 |
| COL1A2 = 0.96 * COL1A1 − 1.91 | 0.953 |
| COL1A2 = 1.11 * COL5A2 + 0.56 | 0.912 |
| COL1A2 = 0.98 * COL3A1 − 1.62 | 0.904 |
| COL1A2 = 1.24 * SPARC − 3.42 | 0.893 |
| COL1A2 = 1.14 * COL5A1 − 0.35 | 0.873 |
| COL1A2 = 1.11 * MMP2 − 0.96 | 0.830 |
| COL1A2 = 1.17 * FBN1 + 0.43 | 0.826 |
| COL1A2 = 1.13 * LOX + 2.13 | 0.824 |
| COL1A2 = 0.86 * SFRP2 + 2.49 | 0.822 |
| COL1A2 = 1.02 * FN1 − 2.07 | 0.810 |
| COL2A1 = 1.57 * COL11A2 − 1.63 | 0.628 |
| COL2A1 = 1.49 * WIF1 − 2.14 | 0.609 |
| COL2A1 = 1.03 * MIA − 2.26 | 0.506 |
| COL3A1 = 0.98 * COL1A1 − 0.29 | 0.942 |
| COL3A1 = 1.14 * COL5A2 + 2.24 | 0.932 |
| COL3A1 = 1.02 * COL1A2 + 1.66 | 0.904 |
| COL3A1 = 1.27 * SPARC − 1.84 | 0.884 |
| COL3A1 = 1.14 * MMP2 + 0.68 | 0.884 |
| COL3A1 = 1.17 * COL5A1 + 1.31 | 0.866 |
| COL3A1 = 1.15 * LOX + 3.85 | 0.845 |
| COL3A1 = 0.88 * SFRP2 + 4.22 | 0.807 |
| COL3A1 = 1.20 * FBN1 + 2.11 | 0.798 |
| COL3A1 = 0.73 * EDIL3 + 9.00 | 0.772 |
| COL5A1 = 0.84 * COL1A1 − 1.37 | 0.891 |
| COL5A1 = 0.87 * COL1A2 + 0.30 | 0.873 |
| COL5A1 = 0.97 * COL5A2 + 0.80 | 0.870 |
| COL5A1 = 0.85 * COL3A1 − 1.12 | 0.866 |
| COL5A1 = 1.09 * SPARC − 2.69 | 0.802 |
| COL5A1 = 0.98 * LOX + 2.17 | 0.801 |
| COL5A1 = 0.89 * FN1 − 1.51 | 0.798 |
| COL5A1 = 1.26 * MMP14 − 3.72 | 0.788 |
| COL5A1 = 0.69 * COL11A1 + 4.46 | 0.774 |
| COL5A1 = 1.06 * THBS2 − 0.29 | 0.766 |
| COL5A2 = 0.88 * COL3A1 − 1.98 | 0.932 |
| COL5A2 = 0.90 * COL1A2 − 0.51 | 0.912 |
| COL5A2 = 0.86 * COL1A1 − 2.23 | 0.901 |
| COL5A2 = 1.03 * COL5A1 − 0.82 | 0.870 |
| COL5A2 = 1.12 * SPARC − 3.60 | 0.860 |
| COL5A2 = 1.00 * MMP2 − 1.38 | 0.847 |
| COL5A2 = 1.02 * LOX + 1.42 | 0.842 |
| COL5A2 = 1.35 * TIMP2 − 4.88 | 0.817 |
| COL5A2 = 0.65 * EDIL3 + 5.95 | 0.807 |
| COL5A2 = 0.72 * COL11A1 + 3.78 | 0.792 |
| COL9A3 = 1.18 * SOX10 − 3.85 | 0.554 |
| COL9A3 = 2.26 * KCNK5 − 10.10 | 0.528 |
| COL9A3 = 1.07 * MIA − 1.86 | 0.495 |
| COX7B = 1.04 * USMG5 − 0.21 | 0.721 |
| COX7B = 1.34 * HSPA8 − 6.80 | 0.694 |
| COX7B = 1.01 * HSPA4 + 0.34 | 0.646 |
| COX7B = 1.35 * PRKAG1 − 1.22 | 0.629 |
| COX7B = 1.17 * EIF4G1 − 2.41 | 0.629 |
| COX7B = 0.98 * TXNL1 + 1.94 | 0.624 |
| CRK = 0.84 * ATF4 + 0.20 | 0.511 |
| CRK = 0.78 * SH3PXD2A + 2.05 | 0.508 |
| CRK = 0.68 * STX1A + 3.47 | 0.459 |
| CRLF2 = 0.92 * MAGEA11 − 0.18 | 0.870 |
| CRLF2 = 0.98 * NODAL + 0.15 | 0.866 |
| CRLF2 = 0.88 * SLC22A7 + 0.81 | 0.863 |
| CRLF2 = 1.19 * STAT4 − 1.16 | 0.862 |
| CRLF2 = 0.93 * KLK3 + 0.53 | 0.861 |
| CRLF2 = 0.92 * SLC3A1 + 0.23 | 0.855 |
| CRLF2 = 0.85 * ESRRB + 1.27 | 0.854 |
| CRLF2 = 1.02 * PTPN5 − 0.45 | 0.853 |
| CRLF2 = 0.91 * OTX2 + 0.96 | 0.851 |
| CRLF2 = 0.99 * LCN1 − 0.83 | 0.851 |
| CRY1 = 0.56 * CTSA + 4.23 | 0.538 |
| CRY1 = 0.40 * HOXA11 + 5.59 | 0.528 |
| CRY1 = 0.38 * HSPB7 + 5.32 | 0.525 |
| CRY1 = 0.33 * PAX3 + 5.86 | 0.521 |
| CRY1 = 0.65 * SOX7 + 2.77 | 0.515 |
| CRY1 = 0.50 * DDX39B + 4.28 | 0.513 |
| CSDE1 = 1.22 * GNAI3 − 1.03 | 0.657 |
| CSDE1 = −0.49 * EPOR + 14.83 | −0.548 |
| CSDE1 = −0.66 * TGFB1 + 16.47 | −0.542 |
| CSDE1 = −1.02 * TEP1 + 20.22 | −0.538 |
| CSDE1 = −0.45 * BCL6 + 14.66 | −0.538 |
| CSDE1 = −0.59 * ANG + 15.69 | −0.535 |
| CXCL1 = 1.31 * CXCL3 − 3.11 | 0.702 |
| CXCL1 = 1.18 * CXCL8 − 2.16 | 0.610 |
| CXCL1 = 1.27 * CXCL2 − 4.65 | 0.549 |
| CXCL1 = 1.20 * CCL20 − 2.12 | 0.548 |
| CXCL1 = 1.01 * EREG − 1.60 | 0.542 |
| CXCL1 = 1.73 * IL1RAP − 7.83 | 0.525 |
| CXCL10 = 1.34 * GBP1 − 4.53 | 0.781 |
| CXCL10 = 1.42 * TAP1 − 3.96 | 0.779 |
| CXCL10 = 1.56 * STAT1 − 8.46 | 0.775 |
| CXCL10 = 1.13 * CCL5 − 0.76 | 0.772 |
| CXCL10 = 1.30 * OASL + 0.28 | 0.738 |
| CXCL10 = 1.52 * HLA_B − 12.31 | 0.733 |
| CXCL10 = 1.28 * OAS1 − 0.54 | 0.730 |
| CXCL10 = 1.61 * APOL3 − 4.71 | 0.729 |
| CXCL10 = 1.17 * ISG15 − 2.94 | 0.718 |
| CXCL10 = 1.15 * MX1 − 2.65 | 0.711 |
| CXCL13 = 1.84 * IL2RG − 9.22 | 0.814 |
| CXCL13 = 1.31 * CXCR3 − 1.93 | 0.798 |
| CXCL13 = 1.74 * CD3D − 7.53 | 0.771 |
| CXCL13 = 1.42 * CD27 − 3.64 | 0.767 |
| CXCL13 = 1.93 * IL2RB − 9.02 | 0.767 |
| CXCL13 = 1.49 * CCL5 − 7.21 | 0.767 |
| CXCL13 = 1.76 * CD2 − 8.09 | 0.759 |
| CXCL13 = 1.49 * GZMB − 5.02 | 0.750 |
| CXCL13 = 1.93 * CD52 − 10.09 | 0.733 |
| CXCL13 = 2.13 * APOL3 − 12.62 | 0.727 |
| CXCL16 = 0.84 * ICAM1 + 2.45 | 0.570 |
| CXCL16 = 0.99 * SOD2 − 2.25 | 0.428 |
| CXCL16 = 1.11 * CD14 − 0.57 | 0.417 |
| CXCL8 = 1.08 * IL1A − 0.38 | 0.715 |
| CXCL8 = 1.23 * ACKR4 − 2.29 | 0.686 |
| CXCL8 = 0.82 * CXCL6 + 1.35 | 0.675 |
| CXCL8 = 0.93 * AURKC + 1.12 | 0.675 |
| CXCL8 = 0.88 * ABCB5 + 0.93 | 0.667 |
| CXCL8 = 0.95 * DPPA2 − 1.72 | 0.665 |
| CXXC4 = 0.87 * ABCG8 + 1.47 | 0.596 |
| CXXC4 = 1.04 * WNT8B + 0.45 | 0.592 |
| CXXC4 = 0.95 * DKK4 + 0.92 | 0.592 |
| CXXC4 = 0.94 * ADRA1A + 0.75 | 0.590 |
| CXXC4 = 0.77 * FGF19 + 2.56 | 0.585 |
| CXXC4 = 1.45 * ATP7B − 3.06 | 0.576 |
| CYP4V2 = 0.44 * ER_171 + 5.19 | 0.509 |
| CYP4V2 = 1.13 * TCL1B − 1.34 | 0.499 |
| CYP4V2 = 1.81 * REST − 11.84 | 0.495 |
| DAAM1 = 0.29 * FOXA1 + 6.43 | 0.443 |
| DAAM1 = 0.33 * SLCO1B1 + 8.13 | 0.435 |
| DAAM1 = 1.03 * MNAT1 + 0.05 | 0.422 |
| DDX58 = 0.72 * ISG15 + 1.40 | 0.824 |
| DDX58 = 0.71 * MX1 + 1.50 | 0.811 |
| DDX58 = 0.79 * OAS1 + 2.81 | 0.769 |
| DDX58 = 0.82 * IFIT2 + 2.03 | 0.757 |
| DDX58 = 0.80 * OASL + 3.34 | 0.732 |
| DDX58 = 0.74 * IFI27 + 1.38 | 0.728 |
| DHX58 = 0.70 * OASL + 2.56 | 0.684 |
| DHX58 = 0.86 * IRF7 + 0.59 | 0.659 |
| DHX58 = 0.72 * IFIT2 + 1.41 | 0.659 |
| DHX58 = 0.69 * OAS1 + 2.11 | 0.642 |
| DHX58 = 1.17 * CD86 − 1.96 | 0.638 |
| DHX58 = 1.27 * CASP1 − 4.18 | 0.625 |
| DIABLO = 0.91 * CAMKK2 + 1.00 | 0.630 |
| DIABLO = 0.72 * ELK1 + 1.13 | 0.477 |
| DIABLO = 0.87 * HMGXB3 + 1.75 | 0.472 |
| DLC1 = 1.14 * PDGFRB − 2.95 | 0.656 |
| DLC1 = 0.94 * PDGFB − 0.73 | 0.630 |
| DLC1 = 0.86 * BMP8A + 1.66 | 0.617 |
| DLC1 = 1.09 * PCOLCE − 2.83 | 0.578 |
| DLC1 = 1.01 * THY1 − 2.09 | 0.575 |
| DLC1 = 0.94 * FLNC + 0.96 | 0.569 |
| DLGAP5 = 1.16 * CDKN3 − 1.35 | 0.711 |
| DLGAP5 = 0.94 * CDC20 − 1.32 | 0.693 |
| DLGAP5 = 1.01 * KIF2C − 0.13 | 0.674 |
| DLGAP5 = 1.06 * HJURP − 0.32 | 0.662 |
| DLGAP5 = 1.24 * MAD2L1 − 2.95 | 0.634 |
| DLGAP5 = 1.07 * BUB1 − 1.52 | 0.631 |
| DLL4 = 0.78 * NOTCH4 + 1.91 | 0.677 |
| DLL4 = 0.92 * PDGFRB − 1.93 | 0.618 |
| DLL4 = 0.88 * HEYL − 0.01 | 0.611 |
| DLL4 = 0.85 * ACKR3 + 0.06 | 0.554 |
| DLL4 = 1.06 * FLT1 − 1.12 | 0.552 |
| DLL4 = 0.82 * CD34 + 0.39 | 0.542 |
| DMD = 1.25 * CKMT2 − 1.05 | 0.533 |
| DMD = 1.16 * FABP7 − 0.23 | 0.522 |
| DMD = 1.05 * MAGEB1 + 0.55 | 0.521 |
| DMD = 1.27 * GNG7 − 1.83 | 0.503 |
| DNAJA1 = 0.70 * MELK + 5.11 | 0.622 |
| DNAJA1 = 0.60 * DDX58 + 5.75 | 0.545 |
| DNAJA1 = 0.91 * HSPA4 + 1.43 | 0.534 |
| DNAJA1 = −0.41 * KLK4 + 13.58 | −0.525 |
| DNAJA1 = −0.75 * F2R + 17.14 | −0.510 |
| DNAJA1 = −0.64 * PRKG1 + 16.01 | −0.508 |
| DNAJB2 = 0.87 * FAM162A + 1.69 | 0.679 |
| DNAJB2 = 0.75 * LRP5 + 3.09 | 0.667 |
| DNAJB2 = 0.70 * XRCC5 + 4.17 | 0.653 |
| DNAJB2 = 0.66 * YY1 + 3.39 | 0.644 |
| DNAJB2 = 0.62 * ARAF + 3.51 | 0.637 |
| DNAJB2 = 0.58 * MMS19 + 4.09 | 0.634 |
| DNAJC10 = 0.81 * HSPE1 + 0.81 | 0.495 |
| DNAJC10 = −0.42 * TNFSF9 + 12.60 | −0.473 |
| DNAJC10 = −0.85 * SUFU + 16.45 | −0.471 |
| DNAJC13 = 0.96 * MGEA5 − 1.52 | 0.546 |
| DNAJC13 = −0.36 * TERT + 11.27 | −0.517 |
| DNAJC13 = 1.22 * GSK3B − 2.84 | 0.506 |
| DNAJC14 = 0.79 * SMUG1 + 2.00 | 0.536 |
| DNAJC14 = 0.32 * ETV4 + 6.11 | 0.532 |
| DNAJC14 = 0.64 * POLR2J + 3.44 | 0.527 |
| DNAJC14 = 0.55 * DUSP8 + 3.55 | 0.526 |
| DNAJC14 = 0.40 * CTSA + 5.50 | 0.508 |
| DNAJC14 = 0.29 * KLK2 + 6.24 | 0.504 |
| DNAJC8 = 1.10 * BAK1 − 0.89 | 0.647 |
| DNAJC8 = 0.43 * CD160 + 7.05 | 0.634 |
| DNAJC8 = 0.45 * WNT16 + 6.84 | 0.625 |
| DNAJC8 = 0.44 * PRL + 7.05 | 0.602 |
| DNAJC8 = 0.45 * DNAJC5B + 6.84 | 0.597 |
| DNAJC8 = 0.53 * RAB6B + 5.93 | 0.593 |
| DUSP6 = 1.05 * STX1A + 0.48 | 0.575 |
| DUSP6 = 1.22 * SPRY4 − 1.67 | 0.569 |
| DUSP6 = 0.57 * TESC + 4.43 | 0.528 |
| DUSP6 = 0.80 * SPRY2 + 2.17 | 0.516 |
| DUSP6 = 0.96 * STK36 + 1.47 | 0.513 |
| E2F3 = 0.68 * CDC20 + 2.45 | 0.531 |
| E2F3 = 1.00 * CTPS1 + 0.29 | 0.522 |
| E2F3 = 0.64 * STMN1 + 2.24 | 0.500 |
| EAF2 = 0.52 * TNFRSF17 + 3.93 | 0.756 |
| EAF2 = 0.44 * CD79A + 4.32 | 0.744 |
| EAF2 = 0.60 * CCR2 + 3.32 | 0.735 |
| EAF2 = 0.36 * IRF4 + 4.98 | 0.730 |
| EAF2 = 0.54 * PIM2 + 2.74 | 0.728 |
| EAF2 = 0.78 * CASP10 + 1.40 | 0.726 |
| EDIL3 = 1.55 * COL5A2 − 9.22 | 0.807 |
| EDIL3 = 1.11 * COL11A1 − 3.36 | 0.794 |
| EDIL3 = 1.37 * COL3A1 − 12.28 | 0.772 |
| EDIL3 = 1.58 * LOX − 7.03 | 0.760 |
| EDIL3 = 1.60 * COL5A1 − 10.50 | 0.758 |
| EDIL3 = 1.40 * COL1A2 − 10.01 | 0.755 |
| EDIL3 = 1.69 * THBS2 − 10.96 | 0.755 |
| EDIL3 = 2.09 * TIMP2 − 16.78 | 0.754 |
| EDIL3 = 1.34 * COL1A1 − 12.68 | 0.748 |
| EDIL3 = 1.74 * SPARC − 14.80 | 0.744 |
| EEF2K = 0.71 * PALB2 + 3.77 | 0.370 |
| EEF2K = −0.34 * RASD1 + 11.44 | −0.319 |
| EEF2K= 0.75 * CCS + 2.14 | 0.319 |
| EGER = −1.19 * E2F5 + 19.81 | −0.423 |
| EGER = 0.65 * CLCA2 + 4.32 | 0.399 |
| EGFR = 1.29 * SEC61G − 6.16 | 0.391 |
| EIF6 = −0.40 * DUSP4 + 12.53 | −0.464 |
| EIF6 = −0.76 * FAM105A + 16.05 | −0.463 |
| EIF6 = −0.58 * AXIN2 + 14.34 | −0.459 |
| ENG = 0.66 * SERPINF1 + 3.11 | 0.556 |
| ENG = 0.75 * PECAM1 + 2.75 | 0.550 |
| ENG = 0.42 * C3 + 5.60 | 0.547 |
| ENG = 0.83 * GRN + 1.32 | 0.533 |
| ENG = −0.80 * FEN1 + 16.76 | −0.526 |
| ENG = 0.85 * TGFBR2 + 1.80 | 0.523 |
| EPCAM = 0.99 * ERBB3 + 0.97 | 0.570 |
| EPCAM = −1.35 * CD40 + 22.07 | −0.541 |
| EPCAM = 1.13 * RAB25 − 1.57 | 0.533 |
| EPCAM = −1.76 * EMP3 + 26.78 | −0.523 |
| EPCAM = −1.48 * SLA + 22.66 | −0.521 |
| EPCAM = −1.04 * IRF8 + 19.20 | −0.515 |
| ER_154 = 1.05 * ER_109 − 0.38 | 0.822 |
| ER_154 = 0.97 * ER_028 − 0.63 | 0.816 |
| ER_154 = 0.92 * ER_013 − 0.16 | 0.807 |
| ER_154 = 1.00 * CYP7A1 − 0.65 | 0.793 |
| ER_154 = 0.93 * CALML6 − 0.58 | 0.788 |
| ER_154 = 1.09 * ER_120 − 0.36 | 0.783 |
| ER_154 = 1.02 * ER_171 + 0.26 | 0.781 |
| ER_154 = 0.95 * GML − 1.02 | 0.780 |
| ER_154 = 1.15 * DNAJB8 − 2.14 | 0.769 |
| ER_154 = 0.93 * SHH − 0.78 | 0.768 |
| ERBB2 = 0.96 * CREB3L4 + 1.49 | 0.408 |
| ERBB2 = 0.76 * FLNA + 0.87 | 0.379 |
| ERBB2 = 0.76 * DBI + 1.06 | 0.378 |
| ETV7 = 0.89 * TAP1 − 0.67 | 0.793 |
| ETV7 = 0.63 * CXCL10 + 1.81 | 0.695 |
| ETV7 = 0.84 * LAG3 + 1.74 | 0.693 |
| ETV7 = 1.19 * IRF1 − 0.79 | 0.687 |
| ETV7 = 0.98 * STAT1 − 3.50 | 0.686 |
| ETV7 = 0.71 * CCL5 + 1.34 | 0.683 |
| EZH2 = 0.92 * TPX2 + 1.81 | 0.591 |
| EZH2 = 0.75 * TOP2A + 2.24 | 0.589 |
| EZH2 = 0.96 * BUB1 + 0.79 | 0.570 |
| EZH2 = 0.72 * ASPM + 3.52 | 0.563 |
| EZH2 = 1.28 * SMC4 − 1.65 | 0.562 |
| EZH2 = 1.11 * MAD2L1 − 0.49 | 0.553 |
| FABP4 = 1.43 * ADIPOQ − 2.39 | 0.746 |
| FABP4 = 2.15 * IGF1 − 9.32 | 0.523 |
| FABP4 = 2.41 * TSPAN7 − 9.16 | 0.514 |
| FABP4 = 1.59 * CCL14 − 3.30 | 0.513 |
| FADD = 1.12 * RPS6KB2 − 0.61 | 0.332 |
| FADD = 0.37 * CCND1 + 5.89 | 0.332 |
| FAF1 = 0.56 * STMN1 + 3.13 | 0.533 |
| FAF1 = 1.08 * GNAI3 − 2.26 | 0.530 |
| FAF1 = 0.86 * CTPS1 + 1.49 | 0.528 |
| FAF1 = −0.42 * CCR3 + 11.75 | −0.526 |
| FAF1 = −0.49 * LAMP5 + 12.70 | −0.525 |
| FAF1 = −0.43 * EOMES + 12.03 | −0.523 |
| FANCG = 0.92 * MELK − 0.89 | 0.586 |
| FANCG = 0.52 * IFT52 + 3.29 | 0.518 |
| FANCG = 1.18 * TOP3A − 2.46 | 0.516 |
| FANCG = 0.73 * PVR + 1.90 | 0.503 |
| FAS = 0.84 * MFNG + 1.06 | 0.630 |
| FAS = 0.78 * GNGT2 + 2.55 | 0.575 |
| FAS = 0.60 * GZMH + 3.13 | 0.575 |
| FAS = 0.67 * TLR9 + 3.21 | 0.574 |
| FAS = 0.77 * TNFSF14 + 1.86 | 0.572 |
| FAS = 0.70 * SNAI3 + 2.77 | 0.569 |
| FASN = 1.38 * ACSL3 − 2.13 | 0.537 |
| FASN = 1.04 * DBI − 1.70 | 0.529 |
| FASN = 0.48 * SPDEF + 7.34 | 0.522 |
| FBXO5 = 0.85 * HJURP + 1.96 | 0.573 |
| FBXO5 = 0.81 * HMGB2 + 0.26 | 0.565 |
| FBXO5 = 0.75 * CDC20 + 1.22 | 0.561 |
| FBXO5 = 1.13 * RACGAP1 − 1.85 | 0.552 |
| FBXO5 = 0.79 * TTK + 2.50 | 0.550 |
| FBXO5 = 0.56 * CDCA7 + 4.22 | 0.550 |
| FBXW11 = 1.08 * NSD1 + 0.19 | 0.570 |
| FBXW11 = 1.14 * PFDN1 − 1.40 | 0.562 |
| FBXW11 = 1.01 * CTNNA1 − 1.06 | 0.456 |
| FGF13 = 0.97 * HSPB2 + 0.53 | 0.485 |
| FGF13 = 1.22 * PLCE1 − 1.56 | 0.470 |
| FGF13 = 0.84 * CRYAB + 1.46 | 0.468 |
| FGF4 = 0.96 * DNTT + 0.88 | 0.805 |
| FGF4 = 1.00 * EGLN2 − 0.66 | 0.785 |
| FGF4 = 1.01 * SLC22A6 + 0.47 | 0.773 |
| FGF4 = 1.00 * ER_067 + 0.54 | 0.772 |
| FGF4 = 0.88 * CCL27 + 0.60 | 0.748 |
| FGF4 = 1.27 * IL27 − 3.04 | 0.743 |
| FGF4 = 0.98 * TBL1Y + 0.84 | 0.733 |
| FGF4 = 1.20 * DNAJB8 − 0.84 | 0.727 |
| FGF4 = 1.04 * CALML6 + 0.54 | 0.726 |
| FGF4 = 1.03 * EFNA2 − 0.79 | 0.725 |
| FGFR3 = 1.23 * WNT9A − 2.30 | 0.510 |
| FGFR3 = 1.48 * FGFRL1 − 6.70 | 0.502 |
| FGFR3 = 1.16 * AHRR + 0.28 | 0.499 |
| FLT3 = 1.27 * CCR4 − 4.86 | 0.747 |
| FLT3 = 1.08 * CD5 − 2.12 | 0.747 |
| FLT3 = 0.98 * CCR7 − 1.07 | 0.735 |
| FLT3 = 1.06 * CCR2 − 2.63 | 0.699 |
| FLT3 = 1.41 * MFNG − 5.20 | 0.695 |
| FLT3 = 1.56 * PIK3R5 − 5.49 | 0.692 |
| FN1 = 0.98 * COL1A2 + 2.04 | 0.810 |
| FN1 = 0.78 * COL11A1 + 6.72 | 0.809 |
| FN1 = 0.94 * COL1A1 + 0.16 | 0.807 |
| FN1 = 1.13 * COL5A1 + 1.70 | 0.798 |
| FN1 = 1.16 * FBN1 + 2.47 | 0.788 |
| FN1 = 1.11 * LOX + 4.14 | 0.768 |
| FN1 = 1.09 * COL5A2 + 2.60 | 0.757 |
| FN1 = 1.42 * MMP14 − 2.49 | 0.750 |
| FN1 = 0.96 * COL3A1 + 0.44 | 0.749 |
| FN1 = 1.22 * SPARC − 1.33 | 0.743 |
| FOSL1 = 0.78 * CXCL8 + 2.36 | 0.538 |
| FOSL1 = 0.77 * S100A2 + 2.56 | 0.494 |
| FOSL1 = 1.07 * FAM64A − 1.72 | 0.489 |
| GADD45G = 0.59 * IL4 + 3.41 | 0.567 |
| GADD45G = 0.55 * DLL3 + 4.00 | 0.551 |
| GADD45G = 0.58 * FGF17 + 3.90 | 0.542 |
| GADD45G = 0.69 * TIE1 + 2.54 | 0.539 |
| GADD45G = 0.61 * FGF21 + 3.55 | 0.528 |
| GADD45G = 0.83 * CHEK1 + 1.85 | 0.517 |
| GBP1 = 1.15 * STAT1 − 2.78 | 0.854 |
| GBP1 = 1.07 * TAP1 + 0.31 | 0.814 |
| GBP1 = 0.75 * CXCL10 + 3.39 | 0.781 |
| GBP1 = 1.15 * HLA_B − 5.98 | 0.767 |
| GBP1 = 1.21 * HLA_A − 6.15 | 0.756 |
| GBP1 = 0.64 * CXCL9 + 4.25 | 0.752 |
| GBP1 = 0.85 * CCL5 + 2.81 | 0.738 |
| GBP1 = 1.21 * APOL3 − 0.25 | 0.735 |
| GBP1 = 1.66 * HLA_E − 9.23 | 0.722 |
| GBP1 = 1.17 * CD74 − 6.81 | 0.720 |
| GBP7 = 1.01 * FASLG + 0.09 | 0.810 |
| GBP7 = 0.91 * IFNG + 0.75 | 0.806 |
| GBP7 = 0.81 * GZMH + 1.44 | 0.786 |
| GBP7 = 1.07 * GNGT2 + 0.53 | 0.751 |
| GBP7 = 0.77 * TSHR + 1.97 | 0.748 |
| GBP7 = 0.88 * ICOS + 0.99 | 0.746 |
| GBP7 = 0.96 * XCL2 − 0.58 | 0.743 |
| GBP7 = 1.16 * GBP2 − 2.33 | 0.737 |
| GBP7 = 0.95 * DPPA4 − 0.43 | 0.733 |
| GBP7 = 1.20 * TNFSF8 − 2.45 | 0.731 |
| GJA1 = 0.74 * COL3A1 − 0.42 | 0.631 |
| GJA1 = 0.85 * MMP2 + 0.09 | 0.611 |
| GJA1 = 1.14 * TIMP2 − 2.93 | 0.598 |
| GJA1 = 0.85 * COL5A2 + 1.23 | 0.583 |
| GJA1 = 0.54 * EDIL3 + 6.27 | 0.581 |
| GJA1 = 0.86 * LOX + 2.42 | 0.555 |
| GLIS3 = 1.09 * SALL4 − 0.98 | 0.695 |
| GLIS3 = 0.79 * IL11 + 2.26 | 0.667 |
| GLIS3 = 0.99 * FGF1 + 0.79 | 0.627 |
| GLIS3 = 0.86 * HOXD1 + 2.17 | 0.613 |
| GLIS3 = 1.16 * NOX4 − 1.89 | 0.613 |
| GLIS3 = 0.88 * RAB6B + 1.33 | 0.612 |
| GMPS = 0.73 * RRM1 + 3.25 | 0.534 |
| GMPS = 0.98 * SMC4 + 1.75 | 0.527 |
| GMPS = 0.81 * ECT2 + 2.45 | 0.517 |
| GNG12 = 0.85 * KCND2 + 2.73 | 0.446 |
| GNG12 = 0.82 * THBS2 − 0.73 | 0.424 |
| GNG12 = 1.09 * PDGFRB − 1.84 | 0.421 |
| GNLY = 1.31 * IL2RB − 2.62 | 0.863 |
| GNLY = 1.24 * PRF1 − 1.31 | 0.831 |
| GNLY = 1.00 * GZMB + 0.09 | 0.824 |
| GNLY = 1.01 * CCL5 − 1.40 | 0.809 |
| GNLY = 1.25 * IL2RG − 2.77 | 0.800 |
| GNLY = 1.15 * GZMA − 0.30 | 0.790 |
| GNLY = 1.21 * CD8A − 1.81 | 0.786 |
| GNLY = 0.97 * CD38 + 0.97 | 0.780 |
| GNLY = 1.42 * CCR5 − 2.98 | 0.779 |
| GNLY = 1.20 * LAG3 − 0.91 | 0.774 |
| GPAM = 0.39 * ADIPOQ + 4.86 | 0.506 |
| GPAM = 0.65 * ABCA9 + 3.21 | 0.477 |
| GPAM = 0.53 * SLC19A3 + 4.81 | 0.473 |
| GPAT2 = 0.82 * UTY + 3.66 | 0.620 |
| GPAT2 = 0.77 * ER_067 + 3.18 | 0.591 |
| GPAT2 = 0.82 * ER_171 + 3.93 | 0.574 |
| GPAT2 = 0.73 * ER_160 + 3.68 | 0.560 |
| GPAT2 = 0.91 * ER_099 + 2.80 | 0.545 |
| GPAT2 = 0.90 * IL22 + 3.01 | 0.544 |
| GPR17 = 1.09 * FLRT1 − 0.30 | 0.870 |
| GPR17 = 1.12 * KLK3 − 1.18 | 0.858 |
| GPR17 = 1.02 * GATA1 − 0.29 | 0.853 |
| GPR17 = 1.15 * GLI1 − 1.28 | 0.839 |
| GPR17 = 1.11 * SLC3A1 − 1.55 | 0.838 |
| GPR17 = 1.11 * MAGEA11 − 2.05 | 0.837 |
| GPR17 = 0.89 * FGF19 + 0.90 | 0.835 |
| GPR17 = 1.14 * IL5RA − 0.71 | 0.834 |
| GPR17 = 1.32 * EPOR − 2.35 | 0.833 |
| GPR17 = 1.06 * FGF21 − 0.41 | 0.832 |
| GRIN2A = 1.26 * TNFRSF10C − 2.87 | 0.801 |
| GRIN2A = 0.81 * HNF1B + 1.86 | 0.785 |
| GRIN2A = 1.24 * CHEK1 − 1.43 | 0.784 |
| GRIN2A = 0.82 * GATA4 + 2.02 | 0.782 |
| GRIN2A = 0.89 * CRYAA + 1.10 | 0.782 |
| GRIN2A = 1.03 * BDNF − 0.21 | 0.779 |
| GRIN2A = 1.05 * PTPN5 − 0.39 | 0.778 |
| GRIN2A = 1.03 * CRP − 0.48 | 0.776 |
| GRIN2A = 0.77 * FGF3 + 2.17 | 0.773 |
| GRIN2A = 0.99 * CCL8 + 0.23 | 0.772 |
| GSN = 0.94 * YY1 + 1.59 | 0.822 |
| GSN = 0.82 * MMS19 + 2.64 | 0.788 |
| GSN = 1.16 * APPBP2 + 0.26 | 0.782 |
| GSN = 0.88 * ARAF + 1.81 | 0.781 |
| GSN = 0.70 * MT2A + 2.41 | 0.777 |
| GSN = 1.13 * MAP7D1 + 0.37 | 0.772 |
| GSN = 0.93 * ATXN1 + 2.27 | 0.769 |
| GSN = 0.51 * ACTB + 3.69 | 0.751 |
| GSN = 0.79 * DNAJC7 + 3.16 | 0.747 |
| GSN = 1.10 * ANAPC2 + 0.08 | 0.726 |
| GSR = 0.63 * FASN + 2.86 | 0.453 |
| GSR = 0.84 * TSC22D3 + 1.02 | 0.450 |
| GSR = 0.30 * SPDEF + 7.51 | 0.403 |
| GSTM1 = 1.63 * CACNG1 − 2.54 | 0.469 |
| GSTM1 = 2.57 * CASP9 − 9.06 | 0.467 |
| GSTM1 = 1.80 * RPA3 − 3.54 | 0.466 |
| GZMB = 1.23 * PRF1 − 1.39 | 0.878 |
| GZMB = 1.30 * IL2RB − 2.70 | 0.863 |
| GZMB = 1.00 * GNLY − 0.09 | 0.824 |
| GZMB = 1.17 * CD3D − 1.69 | 0.809 |
| GZMB = 1.42 * CXCR6 − 3.16 | 0.808 |
| GZMB = 1.24 * IL2RG − 2.83 | 0.797 |
| GZMB = 1.18 * CD2 − 2.07 | 0.792 |
| GZMB = 1.37 * CTLA4 − 2.13 | 0.791 |
| GZMB = 1.20 * CD8A − 1.87 | 0.789 |
| GZMB = 1.38 * TBX21 − 1.01 | 0.783 |
| HDAC8 = 1.17 * SETD2 − 3.99 | 0.712 |
| HDAC8 = 1.17 * MAT2A − 5.28 | 0.629 |
| HDAC8 = 0.74 * CCT6A − 0.07 | 0.596 |
| HDAC8 = 1.34 * ATRX − 2.93 | 0.518 |
| HDAC8 = 1.67 * FUS − 10.24 | 0.513 |
| HERPUD1 = 0.67 * XBP1 + 3.48 | 0.681 |
| HERPUD1 = 0.70 * BTG2 + 4.40 | 0.638 |
| HERPUD1 = 0.34 * IRF4 + 9.01 | 0.623 |
| HERPUD1 = 0.42 * CD79A + 8.39 | 0.609 |
| HERPUD1 = 0.37 * SLAMF7 + 8.39 | 0.586 |
| HERPUD1 = 0.42 * CD27 + 8.34 | 0.581 |
| HEY2 = 1.86 * CAPN5 − 6.65 | 0.447 |
| HEY2 = 0.97 * FRZB + 0.65 | 0.447 |
| HEY2 = 1.32 * CDH5 − 1.92 | 0.446 |
| HIC1 = 1.18 * PPP3R2 − 3.06 | 0.703 |
| HIC1 = 0.85 * CACNA2D2 + 1.65 | 0.692 |
| HIC1 = 1.78 * GNG11 − 8.04 | 0.677 |
| HIC1 = 0.79 * EFNA2 + 1.64 | 0.675 |
| HIC1 = 1.13 * HSPB8 − 2.51 | 0.669 |
| HIC1 = 1.13 * IL4 − 2.43 | 0.669 |
| HIST1H3H = 1.36 * RRM2 − 2.94 | 0.622 |
| HIST1H3H = 2.05 * NASP − 10.98 | 0.584 |
| HIST1H3H = 1.54 * MKI67 − 2.85 | 0.568 |
| HIST1H3H = 1.45 * HMGB2 − 4.19 | 0.559 |
| HIST1H3H = 1.53 * CCNB1 − 4.26 | 0.558 |
| HIST1H3H = 1.39 * CKS2 − 3.35 | 0.552 |
| HLA_A = 0.95 * HLA_B + 0.14 | 0.832 |
| HLA_A = 0.88 * TAP1 + 5.34 | 0.778 |
| HLA_A = 0.83 * GBP1 + 5.08 | 0.756 |
| HLA_A = 0.95 * STAT1 + 2.79 | 0.743 |
| HLA_A = 1.00 * CTSS + 3.70 | 0.736 |
| HLA_A = 1.37 * HLA_E − 2.55 | 0.724 |
| HLA_A = 0.96 * CD74 − 0.55 | 0.704 |
| HLA_B = 1.05 * HLA_A − 0.15 | 0.832 |
| HLA_B = 1.44 * HLA_E − 2.83 | 0.811 |
| HLA_B = 0.87 * GBP1 + 5.20 | 0.767 |
| HLA_B = 0.93 * TAP1 + 5.47 | 0.765 |
| HLA_B = 1.01 * CD74 − 0.72 | 0.756 |
| HLA_B = 0.95 * CYBB + 4.76 | 0.741 |
| HLA_B = 1.00 * STAT1 + 2.78 | 0.741 |
| HLA_B = 1.05 * CTSS + 3.74 | 0.738 |
| HLA_B = 0.66 * CXCL10 + 8.08 | 0.733 |
| HLA_B = 1.06 * APOL3 + 4.98 | 0.700 |
| HLA_E = 0.70 * CD74 + 1.46 | 0.827 |
| HLA_E = 0.69 * HLA_B + 1.96 | 0.811 |
| HLA_E = 0.73 * CTSS + 4.55 | 0.796 |
| HLA_E = 0.85 * CD4 + 4.78 | 0.793 |
| HLA_E = 0.66 * CYBB + 5.26 | 0.783 |
| HLA_E = 0.73 * APOL3 + 5.41 | 0.780 |
| HLA_E = 0.75 * FGL2 + 4.42 | 0.779 |
| HLA_E = 0.99 * JAK2 + 2.88 | 0.762 |
| HLA_E = 0.38 * CXCL9 + 8.13 | 0.745 |
| HLA_E = 0.45 * CXCR3 + 9.10 | 0.744 |
| HMGB3 = 1.30 * CDC34 − 3.19 | 0.442 |
| HMGB3 = 1.28 * CRY1 − 1.61 | 0.434 |
| HMGB3 = −0.89 * TNFRSF1B + 16.33 | −0.425 |
| HMOX1 = 1.14 * CTSB − 3.00 | 0.448 |
| HMOX1 = 1.13 * MSR1 − 1.48 | 0.441 |
| HMOX1 = 0.89 * CD163 + 0.33 | 0.432 |
| HRK = 0.78 * FGF6 + 1.72 | 0.720 |
| HRK = 0.77 * RXRG + 1.50 | 0.650 |
| HRK = 1.02 * CCL26 + 0.16 | 0.645 |
| HRK = 0.89 * DPPA3 − 0.44 | 0.636 |
| HRK = 1.07 * DPPA4 − 2.17 | 0.636 |
| HRK = 0.87 * DNAJB13 + 1.10 | 0.632 |
| HSPA1A = 1.05 * HSPA1B + 1.41 | 0.566 |
| HSPA1A = −1.84 * REL + 26.88 | −0.391 |
| HSPA1A = −1.69 * CYLD + 24.86 | −0.389 |
| HSPA1L = 0.66 * ER_109 + 2.22 | 0.683 |
| HSPA1L = 0.72 * ER_120 + 2.19 | 0.671 |
| HSPA1L = 0.71 * ER_013 + 1.86 | 0.669 |
| HSPA1L = 0.67 * ER_067 + 1.77 | 0.669 |
| HSPA1L = 0.69 * ER_154 + 2.29 | 0.646 |
| HSPA1L = 0.62 * SLC22A6 + 1.88 | 0.645 |
| ID1 = 1.21 * ID3 − 2.38 | 0.698 |
| ID1 = 1.20 * PDGFA − 0.64 | 0.490 |
| ID1 = 0.68 * SFRP2 + 1.07 | 0.422 |
| ID2 = −0.90 * UQCRFS1 + 17.96 | −0.392 |
| ID2 = −1.13 * DDX10 + 18.78 | −0.390 |
| ID2 = 0.42 * VCAN + 5.80 | 0.325 |
| IDH1 = 0.88 * RHOA − 0.36 | 0.504 |
| IDH1 = 0.96 * SOD1 − 1.59 | 0.492 |
| IDH1 = 0.89 * FTH1 − 3.80 | 0.488 |
| IDH2 = −1.50 * TRAF3 + 23.22 | −0.511 |
| IDH2 = −0.76 * WNT10A + 16.55 | −0.496 |
| IDH2 = 1.25 * COX7B − 2.84 | 0.494 |
| IDO1 = 1.47 * APOL3 − 6.14 | 0.743 |
| IDO1 = 1.29 * TAP1 − 5.43 | 0.734 |
| IDO1 = 1.39 * STAT1 − 9.18 | 0.693 |
| IDO1 = 1.70 * IRF1 − 5.35 | 0.692 |
| IDO1 = 1.02 * GZMB − 0.91 | 0.690 |
| IDO1 = 1.33 * IL2RB − 3.66 | 0.689 |
| IFI27 = 0.97 * ISG15 + 0.03 | 0.818 |
| IFI27 = 1.06 * OAS1 + 1.93 | 0.801 |
| IFI27 = 0.96 * MX1 + 0.17 | 0.792 |
| IFI27 = 1.35 * DDX58 − 1.86 | 0.728 |
| IFI27 = 1.11 * IFIT2 + 0.89 | 0.726 |
| IFI27 = 1.08 * OASL + 2.65 | 0.713 |
| IFI27 = 0.83 * CXCL10 + 2.41 | 0.709 |
| IFI27 = 1.48 * TYMP − 6.65 | 0.701 |
| IFNA2 = 1.07 * IL2 − 1.37 | 0.848 |
| IFNA2 = 1.20 * SCN3A − 1.41 | 0.843 |
| IFNA2 = 1.02 * RSPO2 − 0.50 | 0.838 |
| IFNA2 = 1.04 * SLC22A2 − 0.79 | 0.838 |
| IFNA2 = 1.05 * GSTA2 − 1.23 | 0.836 |
| IFNA2 = 1.08 * DNAJB7 − 1.09 | 0.834 |
| IFNA2 = 0.99 * IFNA5 − 0.40 | 0.834 |
| IFNA2 = 1.05 * FGF14 − 0.74 | 0.834 |
| IFNA2 = 0.98 * IL17F − 0.66 | 0.828 |
| IFNA2 = 1.02 * CYP3A4 − 0.67 | 0.828 |
| IFNA5 = 0.91 * IFNW1 + 0.99 | 0.914 |
| IFNA5 = 1.01 * APCS − 0.40 | 0.889 |
| IFNA5 = 0.99 * ITLN2 + 0.04 | 0.888 |
| IFNA5 = 1.11 * IFNB1 − 1.08 | 0.887 |
| IFNA5 = 0.97 * OR10J3 − 0.08 | 0.883 |
| IFNA5 = 1.00 * IL17A + 0.95 | 0.876 |
| IFNA5 = 1.03 * PLG − 0.06 | 0.876 |
| IFNA5 = 1.29 * DPPA4 − 3.70 | 0.875 |
| IFNA5 = 1.12 * DPPA2 − 3.31 | 0.871 |
| IFNA5 = 0.99 * CRP − 0.10 | 0.870 |
| IFNAR1 = 0.86 * IRF6 − 1.11 | 0.792 |
| IFNAR1 = 1.09 * XRCC2 − 1.34 | 0.748 |
| IFNAR1 = 0.56 * HNF1A + 5.47 | 0.740 |
| IFNAR1 = 1.15 * GCLM + 1.07 | 0.727 |
| IFNAR1 = 0.66 * DPPA5 + 5.24 | 0.681 |
| IFNAR1 = 0.71 * EPOR + 5.68 | 0.668 |
| IFNW1 = 1.10 * IFNA5 − 1.08 | 0.914 |
| IFNW1 = 1.08 * ITLN2 − 1.04 | 0.902 |
| IFNW1 = 1.10 * APCS − 1.52 | 0.883 |
| IFNW1 = 1.05 * S100A8 − 0.99 | 0.873 |
| IFNW1 = 1.02 * NPPB − 0.02 | 0.873 |
| IFNW1 = 1.07 * OR10J3 − 1.19 | 0.873 |
| IFNW1 = 1.41 * DPPA4 − 5.13 | 0.870 |
| IFNW1 = 1.13 * PLG − 1.15 | 0.869 |
| IFNW1 = 0.92 * SLC28A2 + 0.42 | 0.865 |
| IFNW1 = 1.02 * RXRG − 0.34 | 0.865 |
| IGFBP7 = 0.70 * TIMP3 + 3.25 | 0.661 |
| IGFBP7 = 0.95 * PDGFRB + 2.67 | 0.652 |
| IGFBP7 = 0.91 * CALD1 + 1.93 | 0.635 |
| IGFBP7 = 0.88 * TIMP2 + 1.19 | 0.627 |
| IGFBP7 = 0.67 * COL5A1 + 3.84 | 0.602 |
| IGFBP7 = 0.59 * COL1A2 + 4.04 | 0.596 |
| IL12A = 0.86 * FGF17 + 0.16 | 0.715 |
| IL12A = 0.80 * DLL3 + 0.34 | 0.714 |
| IL12A = 0.99 * SOCS2 − 1.04 | 0.712 |
| IL12A = 0.85 * CSF2 − 0.01 | 0.710 |
| IL12A = 0.84 * TNNI3 − 0.31 | 0.707 |
| IL12A = 0.83 * UGT1A1 + 0.16 | 0.706 |
| IL12A = 0.76 * C1orf159 + 1.75 | 0.700 |
| IL6R = 0.72 * TBX21 + 3.98 | 0.565 |
| IL6R = 0.78 * MAP4K1 + 2.52 | 0.556 |
| IL6R = 0.67 * CCR2 + 3.93 | 0.545 |
| IL6R = −1.13 * SERPINH1 + 21.92 | −0.541 |
| IL6R = 0.72 * CTLA4 + 3.40 | 0.541 |
| IL6R = 0.58 * TNFRSF17 + 4.61 | 0.540 |
| INHBA = 0.94 * COL5A2 − 0.86 | 0.737 |
| INHBA = 0.97 * COL5A1 − 1.62 | 0.737 |
| INHBA = 0.67 * COL11A1 + 2.72 | 0.734 |
| INHBA = 0.96 * LOX + 0.47 | 0.695 |
| INHBA = 0.86 * FN1 − 3.07 | 0.692 |
| INHBA = 0.61 * EDIL3 + 4.76 | 0.672 |
| IRF1 = 0.71 * CD2 + 1.37 | 0.794 |
| IRF1 = 0.58 * CD38 + 3.15 | 0.790 |
| IRF1 = 0.78 * IL2RB + 1.00 | 0.788 |
| IRF1 = 0.79 * CD274 + 1.86 | 0.781 |
| IRF1 = 0.74 * IL2RG + 0.92 | 0.768 |
| IRF1 = 0.85 * CCR5 + 0.75 | 0.766 |
| IRF1 = 0.90 * FOXP3 + 2.30 | 0.754 |
| IRF1 = 0.74 * PRF1 + 1.78 | 0.752 |
| IRF1 = 0.85 * CXCR6 + 0.72 | 0.750 |
| IRF1 = 0.71 * PDCD1 + 2.84 | 0.749 |
| IRF2 = 0.64 * IRF1 + 3.39 | 0.608 |
| IRF2 = 0.74 * TLR3 + 2.80 | 0.607 |
| IRF2 = 0.50 * CD274 + 4.63 | 0.574 |
| IRF2 = 0.52 * TBX21 + 4.71 | 0.573 |
| IRF2 = 0.72 * PIK3R5 + 3.36 | 0.573 |
| IRF2 = 0.81 * CASP1 + 0.53 | 0.555 |
| IRF4 = 1.50 * PIM2 − 6.18 | 0.885 |
| IRF4 = 1.08 * SLAMF7 − 1.83 | 0.882 |
| IRF4 = 1.23 * CD27 − 1.96 | 0.870 |
| IRF4 = 1.22 * CD79A − 1.82 | 0.851 |
| IRF4 = 1.25 * CD38 − 2.03 | 0.833 |
| IRF4 = 2.15 * CASP10 − 9.88 | 0.796 |
| IRF4 = 1.13 * CXCR3 − 0.49 | 0.779 |
| IRF4 = 1.43 * TNFRSF17 − 2.92 | 0.773 |
| IRF4 = 1.94 * IL10RA − 10.69 | 0.773 |
| IRF4 = 1.59 * IL2RG − 6.80 | 0.756 |
| IRF7 = 0.82 * OASL + 2.29 | 0.785 |
| IRF7 = 0.80 * OAS1 + 1.77 | 0.752 |
| IRF7 = 0.84 * IFIT2 + 0.95 | 0.722 |
| IRF7 = 0.72 * MX1 + 0.44 | 0.708 |
| IRF7 = 0.84 * LAG3 + 2.02 | 0.669 |
| IRF7 = 1.01 * APOL3 − 0.86 | 0.668 |
| IRF9 = 0.55 * OAS1 + 4.83 | 0.684 |
| IRF9 = 0.49 * MX1 + 3.92 | 0.677 |
| IRF9 = 0.94 * HLA_E − 2.05 | 0.676 |
| IRF9 = 0.69 * APOL3 + 3.04 | 0.675 |
| IRF9 = 0.65 * HLA_B − 0.21 | 0.671 |
| IRF9 = 0.57 * GBP1 + 3.18 | 0.660 |
| IRS1 = 1.24 * DLC1 − 1.57 | 0.499 |
| IRS1 = 1.14 * PLCB1 − 1.68 | 0.458 |
| IRS1 = −2.69 * PPP2CA + 37.98 | −0.428 |
| ISG15 = 0.99 * MX1 + 0.15 | 0.922 |
| ISG15 = 1.10 * OAS1 + 1.96 | 0.861 |
| ISG15 = 1.15 * IFIT2 + 0.85 | 0.850 |
| ISG15 = 1.39 * DDX58 − 1.95 | 0.824 |
| ISG15 = 1.03 * IFI27 − 0.03 | 0.818 |
| ISG15 = 1.12 * OASL + 2.68 | 0.790 |
| ISG15 = 1.53 * TYMP −6.89 | 0.763 |
| ISG15 = 1.31 * STAT1 − 4.50 | 0.743 |
| ISG15 = 0.85 * CXCL10 + 2.50 | 0.718 |
| ITGA2 = −0.89 * CD8A + 14.95 | −0.399 |
| ITGA2 = −0.98 * CD274 + 14.58 | −0.395 |
| ITGA2 = 1.89 * ITGB1 − 16.08 | 0.395 |
| ITGB7 = 2.77 * TOP3A − 15.76 | 0.659 |
| ITGB7 = 1.33 * IFT52 − 3.15 | 0.655 |
| ITGB7 = 2.02 * PRKACA − 11.38 | 0.611 |
| ITGB7 = 1.39 * CD47 − 4.67 | 0.593 |
| ITGB7 = 2.25 * PML − 15.04 | 0.583 |
| ITGB7 = 1.62 * CCR8 − 2.96 | 0.581 |
| ITPKB = 0.62 * BOC + 4.77 | 0.447 |
| ITPKB = 0.62 * ITGA6 + 3.88 | 0.413 |
| ITPKB = 0.69 * PLCB4 + 3.95 | 0.404 |
| JAG1 = 1.33 * FRMD6 − 2.64 | 0.496 |
| JAG1 = 1.18 * HEYL + 0.74 | 0.493 |
| JAG1 = 1.24 * PDGFRB − 1.88 | 0.481 |
| JAK1 = 0.80 * IL6ST + 2.67 | 0.538 |
| JAK1 = −0.60 * PRC1 + 15.87 | −0.484 |
| JAK1 = 0.92 * MGEA5 + 0.56 | 0.470 |
| JAK2 = 0.76 * FGL2 + 1.55 | 0.780 |
| JAK2 = 0.74 * CTSS + 1.69 | 0.765 |
| JAK2 = 1.01 * HLA_E − 2.90 | 0.762 |
| JAK2 = 0.71 * CD74 − 1.43 | 0.756 |
| JAK2 = 0.51 * CCL5 + 4.41 | 0.739 |
| JAK2 = 0.64 * IL2RG + 3.72 | 0.738 |
| JAK2 = 0.62 * CD8A + 4.20 | 0.732 |
| JAK2 = 1.00 * CD86 + 1.00 | 0.730 |
| JAK2 = 0.86 * CD4 + 1.85 | 0.722 |
| JAK2 = 0.66 * CYBB + 2.40 | 0.718 |
| JPH3 = 0.87 * GATA4 + 1.52 | 0.822 |
| JPH3 = 0.95 * TNNI3 + 0.39 | 0.812 |
| JPH3 = 0.96 * WNT7A + 0.82 | 0.808 |
| JPH3 = 0.99 * SLC3A1 − 0.27 | 0.806 |
| JPH3 = 0.94 * FGF17 + 1.09 | 0.806 |
| JPH3 = 0.96 * CHGA + 1.03 | 0.806 |
| JPH3 = 0.95 * CEBPE + 0.90 | 0.804 |
| JPH3 = 0.92 * HSPA2 + 1.31 | 0.803 |
| JPH3 = 0.91 * ESRRB + 0.86 | 0.798 |
| JPH3 = 1.11 * SOCS2 − 0.33 | 0.795 |
| KCNK5 = 0.48 * MIA + 3.56 | 0.586 |
| KCNK5 = 0.53 * SOX10 + 2.68 | 0.577 |
| KCNK5 = 1.20 * LRP6 − 3.12 | 0.575 |
| KCNK5 = 1.21 * FOXC1 − 1.14 | 0.558 |
| KCNK5 = 0.44 * COL9A3 + 4.47 | 0.528 |
| KDM1A = 0.61 * STMN1 + 2.71 | 0.568 |
| KDM1A = 0.95 * CCT3 − 1.17 | 0.527 |
| KDM1A = 0.55 * MYC + 3.56 | 0.523 |
| KDM1A = 0.74 * PRKDC + 2.27 | 0.505 |
| KDM6A = 1.17 * ZFX − 2.78 | 0.524 |
| KDM6A = 0.85 * CASP8 + 1.65 | 0.495 |
| KDM6A = 0.82 * PRKACB + 1.64 | 0.474 |
| KDR = 0.92 * RAMP2 + 0.82 | 0.653 |
| KDR = 0.87 * CD34 + 1.29 | 0.651 |
| KDR = 1.12 * FLT1 − 0.29 | 0.648 |
| KDR = 0.84 * NOTCH4 + 2.88 | 0.586 |
| KDR = 0.79 * TEK + 3.27 | 0.536 |
| KDR = 0.96 * PPAP2A − 0.54 | 0.528 |
| KIF3B = −0.23 * ER_120 + 9.60 | −0.471 |
| KIF3B = −0.43 * CENPN + 11.72 | −0.457 |
| KIF3B = −0.70 * ATF4 + 16.13 | −0.434 |
| KNTC1 = 0.70 * HELLS + 3.65 | 0.481 |
| KNTC1 = 0.71 * HJURP + 3.68 | 0.439 |
| KNTC1 = 0.81 * MAD2L1 + 2.10 | 0.427 |
| KRT18 = 0.59 * TNR + 7.90 | 0.586 |
| KRT18 = 0.48 * SLC10A1 + 9.00 | 0.569 |
| KRT18 = 0.55 * KLB + 8.49 | 0.568 |
| KRT18 = 0.50 * S100A7A + 8.26 | 0.565 |
| KRT18 = 0.76 * NANOG + 5.06 | 0.549 |
| KRT18 = 0.48 * MAOA + 9.10 | 0.546 |
| KRT7 = 0.97 * OCLN + 5.80 | 0.669 |
| KRT7 = 0.84 * KRT19 + 1.64 | 0.642 |
| KRT7 = 1.76 * KRT8 − 10.44 | 0.617 |
| KRT7 = 2.19 * CAPN1 − 10.20 | 0.561 |
| KRT7 = 1.55 * CDH1 − 4.74 | 0.529 |
| KRT7 = 1.40 * RAB25 − 1.37 | 0.510 |
| LAG3 = 1.03 * PRF1 − 0.33 | 0.788 |
| LAG3 = 0.97 * OASL + 0.31 | 0.785 |
| LAG3 = 1.08 * IL2RB − 1.42 | 0.784 |
| LAG3 = 1.00 * CD8A − 0.74 | 0.783 |
| LAG3 = 0.83 * GNLY + 0.75 | 0.774 |
| LAG3 = 0.84 * CCL5 − 0.40 | 0.757 |
| LAG3 = 1.11 * CD274 − 0.36 | 0.748 |
| LAG3 = 1.26 * SOCS1 − 4.14 | 0.743 |
| LAG3 = 1.18 * CXCR6 − 1.81 | 0.741 |
| LAG3 = 0.84 * GZMB + 0.83 | 0.735 |
| LCN2 = 1.51 * CCL28 − 4.04 | 0.470 |
| LCN2 = 1.73 * PROM1 − 10.31 | 0.416 |
| LCN2 = 1.95 * OCLN − 5.17 | 0.409 |
| LFNG = 0.63 * JAK3 + 3.56 | 0.624 |
| LFNG = 1.11 * ATM − 0.63 | 0.552 |
| LFNG = 0.79 * BATF + 2.33 | 0.549 |
| LFNG = 1.01 * LAT − 0.49 | 0.546 |
| LFNG = 0.53 * CD19 + 5.46 | 0.536 |
| LFNG = 0.80 * GPR160 + 2.04 | 0.535 |
| LIF = 1.30 * F3 − 2.03 | 0.524 |
| LIF = 1.03 * CXCL8 + 0.87 | 0.480 |
| LIF = 1.19 * CLCF1 − 0.50 | 0.473 |
| LOX = 0.87 * COL3A1 − 3.34 | 0.845 |
| LOX = 0.98 * COL5A2 − 1.39 | 0.842 |
| LOX = 0.89 * COL1A2 − 1.89 | 0.824 |
| LOX = 0.85 * COL1A1 − 3.59 | 0.819 |
| LOX = 1.02 * COL5A1 − 2.20 | 0.801 |
| LOX = 1.10 * SPARC − 4.93 | 0.798 |
| LOX = 0.90 * FN1 − 3.74 | 0.768 |
| LOX = 0.99 * MMP2 − 2.75 | 0.764 |
| LOX = 0.63 * EDIL3 + 4.46 | 0.760 |
| LOX = 1.33 * TIMP2 − 6.19 | 0.715 |
| LOXL1 = 1.13 * TIMP2 − 4.31 | 0.705 |
| LOXL1 = 0.87 * COL5A1 − 0.90 | 0.696 |
| LOXL1 = 1.23 * PDGFRB − 2.40 | 0.696 |
| LOXL1 = 0.84 * COL5A2 − 0.20 | 0.686 |
| LOXL1 = 0.76 * COL1A2 − 0.63 | 0.681 |
| LOXL1 = 0.65 * SFRP2 + 1.26 | 0.669 |
| LRIG1 = 0.90 * CXCR4 + 0.35 | 0.445 |
| LRIG1 = −0.84 * LGALS1 + 19.65 | −0.358 |
| LRIG1 = 1.26 * KIF3A − 0.34 | 0.356 |
| LRP12 = 0.88 * FZD6 + 0.51 | 0.523 |
| LRP12 = −0.98 * BLVRA + 17.83 | −0.464 |
| LRP12 = −0.77 * CASP10 + 14.80 | −0.445 |
| LYVE1 = 0.85 * WNT16 + 1.68 | 0.839 |
| LYVE1 = 0.74 * PPP2R2B + 2.79 | 0.835 |
| LYVE1 = 0.83 * PLG + 1.87 | 0.831 |
| LYVE1 = 0.68 * SLC28A2 + 3.03 | 0.828 |
| LYVE1 = 0.82 * CYP3A5 + 1.35 | 0.826 |
| LYVE1 = 0.81 * DPPA5 + 1.35 | 0.825 |
| LYVE1 = 0.91 * DPPA2 − 0.76 | 0.821 |
| LYVE1 = 0.82 * RND2 + 2.16 | 0.820 |
| LYVE1 = 0.74 * IL12B + 2.84 | 0.817 |
| LYVE1 = 0.93 * SLC25A4 + 0.79 | 0.815 |
| MAD2L1 = 0.89 * CCNA2 + 1.16 | 0.819 |
| MAD2L1 = 1.03 * PLK4 − 0.03 | 0.660 |
| MAD2L1 = 0.87 * CCNB1 + 0.31 | 0.658 |
| MAD2L1 = 0.81 * DLGAP5 + 2.39 | 0.634 |
| MAD2L1 = 1.15 * RACGAP1 − 1.81 | 0.629 |
| MAD2L1 = 0.86 * HJURP + 2.07 | 0.622 |
| MADD = 0.49 * NR1H3 + 4.84 | 0.476 |
| MADD = 0.69 * MAP3K14 + 3.04 | 0.464 |
| MADD = 0.33 * PDCD1 + 6.31 | 0.461 |
| MAP3K4 = 1.06 * ARID1B − 1.77 | 0.508 |
| MAP3K4 = 0.93 * FGFR1OP + 1.25 | 0.495 |
| MAP3K4 = 0.77 * C1orf86 + 3.55 | 0.373 |
| MAP3K5 = 0.48 * IL20RA + 5.54 | 0.503 |
| MAP3K5 = 0.67 * ABCC3 + 3.11 | 0.476 |
| MAP3K5 = −0.72 * RAD21 + 16.48 | −0.453 |
| MAPK10 = 1.06 * CKMT2 + 0.74 | 0.593 |
| MAPK10 = 0.95 * AR + 1.18 | 0.543 |
| MAPK10 = 0.96 * IL20RA + 1.56 | 0.535 |
| MAPK10 = 0.88 * EGF + 1.81 | 0.532 |
| MAPK10 = 0.85 * CXXC4 + 2.17 | 0.531 |
| MAPK10 = 0.78 * PLA2G4F + 3.84 | 0.514 |
| MAPK3 = 1.08 * SH2B1 − 0.78 | 0.468 |
| MAPK3 = 1.18 * NUMB − 1.09 | 0.462 |
| MAPK3 = 0.83 * ATP6V0C + 0.31 | 0.456 |
| MAT2A = 0.62 * CCT6A + 4.63 | 0.726 |
| MAT2A = 0.98 * SETD2 + 1.28 | 0.685 |
| MAT2A = 0.85 * HDAC8 + 4.50 | 0.629 |
| MAT2A = 0.80 * TPI1 + 1.79 | 0.530 |
| MAT2A = 0.97 * PPID + 2.85 | 0.509 |
| MAX = 0.41 * FGL2 + 4.82 | 0.569 |
| MAX = 0.36 * CYBB + 5.28 | 0.561 |
| MAX = 0.38 * CD74 + 3.21 | 0.533 |
| MAX = 0.54 * HLA_E + 2.42 | 0.519 |
| MAX = 0.33 * PDCD1LG2 + 6.67 | 0.517 |
| MAX = 0.40 * APOL3 + 5.36 | 0.511 |
| MCM5 = 0.73 * GTSE1 + 4.53 | 0.649 |
| MCM5 = 0.98 * MCM3 + 0.62 | 0.610 |
| MCM5 = 1.06 * DNMT1 − 0.46 | 0.593 |
| MCM5 = 0.86 * MCM6 + 2.49 | 0.591 |
| MCM5 = 0.79 * HMGB2 + 2.15 | 0.587 |
| MCM5 = 1.12 * NASP − 1.55 | 0.578 |
| MCM6 = 1.26 * DNMT1 − 3.81 | 0.601 |
| MCM6 = 1.16 * MCM5 − 2.88 | 0.591 |
| MCM6 = 1.76 * RIF1 − 8.15 | 0.589 |
| MCM6 = 0.98 * RRM1 − 0.22 | 0.552 |
| MCM6 = 0.74 * ASPM + 3.06 | 0.548 |
| MCM6 = 1.29 * NASP − 4.68 | 0.548 |
| MED12 = 0.67 * STAG2 + 2.26 | 0.394 |
| MED12 = 0.69 * ATRX + 3.13 | 0.393 |
| MED12 = 0.64 * AIFM1 + 3.27 | 0.377 |
| MESP1 = 0.73 * AGT + 1.49 | 0.475 |
| MESP1 = 1.43 * HSP90AA1 − 2.94 | 0.475 |
| MESP1 = 1.53 * FES − 3.96 | 0.474 |
| MGEA5 = 0.95 * STAG2 + 1.73 | 0.587 |
| MGEA5 = 1.03 * BIRC6 + 0.64 | 0.573 |
| MGEA5 = 1.15 * MDM4 − 0.86 | 0.552 |
| MGEA5 = 1.04 * DNAJC13 + 1.58 | 0.546 |
| MGEA5 = 0.90 * KIF3A + 3.85 | 0.512 |
| MGEA5 = −0.55 * IL1B + 14.87 | −0.509 |
| MIXL1 = 0.84 * MPO + 1.87 | 0.840 |
| MIXL1 = 1.08 * TSHR − 0.88 | 0.821 |
| MIXL1 = 0.95 * PRL + 0.49 | 0.820 |
| MIXL1 = 1.08 * ABCB5 − 0.61 | 0.809 |
| MIXL1 = 0.92 * SLC10A1 + 0.99 | 0.806 |
| MIXL1 = 1.18 * DPPA2 − 3.90 | 0.794 |
| MIXL1 = 1.01 * S100A8 − 0.33 | 0.790 |
| MIXL1 = 0.99 * AQP7 − 1.77 | 0.789 |
| MIXL1 = 0.92 * PTPRR + 1.31 | 0.788 |
| MIXL1 = 1.05 * IL17A + 0.58 | 0.787 |
| MLLT3 = 1.67 * RECQL5 − 6.27 | 0.412 |
| MLLT3 = 0.52 * ER_109 + 5.00 | 0.393 |
| MLLT3 = 1.76 * GTF2H3 − 6.29 | 0.348 |
| MLPH = 0.71 * FOXA1 + 2.50 | 0.690 |
| MLPH = 1.35 * FMO5 − 1.44 | 0.608 |
| MLPH = 0.94 * TMEM45B + 2.19 | 0.571 |
| MLPH = 0.91 * LRG1 + 2.24 | 0.565 |
| MLPH = 1.14 * HOXA9 + 0.01 | 0.564 |
| MLPH = 1.00 * HMGCS2 + 1.05 | 0.563 |
| MME = 1.10 * GLIS3 + 0.06 | 0.564 |
| MME = 1.45 * FRMD6 − 5.20 | 0.548 |
| MME = 0.87 * CA12 + 2.42 | 0.521 |
| MME = 0.79 * SPINK1 + 3.74 | 0.518 |
| MME = 1.98 * BNIP3L − 10.35 | 0.511 |
| MME = 1.08 * FGF1 + 1.00 | 0.500 |
| MMP14 = 0.77 * COL5A2 + 3.58 | 0.789 |
| MMP14 = 0.79 * COL5A1 + 2.95 | 0.788 |
| MMP14 = 0.70 * FN1 + 1.75 | 0.750 |
| MMP14 = 0.68 * COL3A1 + 2.06 | 0.744 |
| MMP14 = 0.69 * COL1A2 + 3.19 | 0.740 |
| MMP14 = 0.66 * COL1A1 + 1.87 | 0.738 |
| MMP14 = 1.03 * TIMP2 − 0.16 | 0.737 |
| MMP14 = 0.47 * MMP11 + 7.01 | 0.721 |
| MMP14 = 1.26 * ITGA5 + 1.00 | 0.721 |
| MMP14 = 0.77 * MMP2 + 2.52 | 0.717 |
| MSH3 = 0.84 * AGGF1 + 1.42 | 0.563 |
| MSH3 = 1.14 * RAD17 − 1.69 | 0.483 |
| MSH3 = 0.91 * CHD1 − 0.08 | 0.475 |
| MSL2 = 0.82 * ATR + 1.65 | 0.581 |
| MSL2 = 0.66 * TFDP2 + 3.51 | 0.471 |
| MSL2 = 0.74 * GMPS + 1.93 | 0.386 |
| MTHFD1 = 0.77 * POLE2 + 2.91 | 0.444 |
| MTHFD1 = 0.73 * HELLS + 2.26 | 0.425 |
| MTHFD1 = 0.69 * DLGAP5 + 2.56 | 0.416 |
| MX1 = 1.01 * ISG15 − 0.15 | 0.922 |
| MX1 = 1.11 * OAS1 + 1.84 | 0.875 |
| MX1 = 1.16 * IFIT2 + 0.72 | 0.841 |
| MX1 = 1.41 * DDX58 − 2.12 | 0.811 |
| MX1 = 1.04 * IFI27 − 0.18 | 0.792 |
| MX1 = 1.13 * OASL + 2.56 | 0.787 |
| MX1 = 1.32 * STAT1 − 4.70 | 0.741 |
| MX1 = 0.87 * CXCL10 + 2.30 | 0.711 |
| MX1 = 1.38 * IRF7 − 0.61 | 0.708 |
| MYBL1 = 2.16 * ARMC1 − 12.73 | 0.577 |
| MYBL1 = 1.73 * RAD21 − 10.58 | 0.548 |
| MYBL1 = 1.43 * GGH − 6.25 | 0.526 |
| MYBL1 = 2.40 * CCT3 − 18.41 | 0.518 |
| MYCN = 0.81 * SOX2 + 1.48 | 0.536 |
| MYCN = 1.19 * TNNC2 − 1.42 | 0.490 |
| MYCN = 1.15 * DDX39B − 1.00 | 0.488 |
| MYOD1 = 1.07 * PLA2G3 − 0.79 | 0.866 |
| MYOD1 = 1.09 * CEACAM3 − 1.14 | 0.853 |
| MYOD1 = 1.10 * CMTM2 − 0.56 | 0.853 |
| MYOD1 = 1.18 * PLA2G10 − 3.58 | 0.851 |
| MYOD1 = 1.51 * RPS6KB1 − 3.65 | 0.837 |
| MYOD1 = 1.38 * TIE1 − 3.55 | 0.834 |
| MYOD1 = 1.05 * PF4V1 − 0.63 | 0.831 |
| MYOD1 = 1.17 * IL4 − 1.72 | 0.825 |
| MYOD1 = 1.01 * SOST − 0.38 | 0.823 |
| MYOD1 = 0.88 * UTF1 + 0.94 | 0.822 |
| NAIP = 1.31 * MSH3 − 0.44 | 0.465 |
| NAIP = 1.05 * ATG7 + 2.28 | 0.456 |
| NAIP = 0.84 * HHEX + 3.57 | 0.435 |
| NAMPT = 0.71 * FASN + 2.15 | 0.445 |
| NAMPT = 0.98 * ACSL3 + 0.63 | 0.429 |
| NAMPT = 1.04 * IDH1 − 0.82 | 0.422 |
| NASP = 0.62 * STMN1 + 3.79 | 0.620 |
| NASP = 0.97 * CTPS1 + 1.88 | 0.611 |
| NASP = 0.97 * DNMT1 + 0.67 | 0.607 |
| NASP = 0.49 * HIST1H3H + 5.37 | 0.584 |
| NASP = 0.90 * MCM5 + 1.39 | 0.578 |
| NASP = 0.67 * CDC20 + 3.99 | 0.566 |
| NCOA2 = 0.83 * CHD7 + 2.21 | 0.591 |
| NCOA2 = 1.14 * CCS − 1.10 | 0.550 |
| NCOA2 = 0.95 * ARMC1 + 0.53 | 0.547 |
| NCOA2 = 0.83 * PRKDC + 1.87 | 0.510 |
| NFKB1 = 0.58 * TNFAIP3 + 4.25 | 0.498 |
| NFKB1 = 0.65 * TIFA + 3.55 | 0.497 |
| NFKB1 = 0.38 * BIRC3 + 5.95 | 0.470 |
| NKD1 = 1.28 * NFATC4 − 5.96 | 0.583 |
| NKD1 = 1.12 * NGF − 4.15 | 0.574 |
| NKD1 = 0.87 * OTX2 − 2.28 | 0.570 |
| NKD1 = 0.85 * NKX2_1 − 2.03 | 0.565 |
| NKD1 = 0.84 * CEBPE − 1.85 | 0.552 |
| NKD1 = 0.85 * IL4 − 2.39 | 0.550 |
| NLRP3 = 0.55 * CRLF2 + 3.89 | 0.800 |
| NLRP3 = 0.62 * EPOR + 3.61 | 0.795 |
| NLRP3 = 0.57 * KNG1 + 3.61 | 0.795 |
| NLRP3 = 0.53 * CEACAM7 + 4.13 | 0.793 |
| NLRP3 = 0.60 * PROK2 + 3.54 | 0.793 |
| NLRP3 = 0.54 * NODAL + 3.99 | 0.791 |
| NLRP3 = 0.56 * CRP + 3.57 | 0.791 |
| NLRP3 = 0.53 * CCL8 + 3.98 | 0.789 |
| NLRP3 = 0.58 * ABCB5 + 3.53 | 0.787 |
| NLRP3 = 0.63 * CXCR2 + 3.37 | 0.785 |
| NMU = 1.10 * ARNT2 − 2.12 | 0.479 |
| NMU = 2.52 * RRM1 − 19.18 | 0.354 |
| NMU = 2.76 * FANCL − 19.72 | 0.345 |
| NOD2 = 0.76 * IL1B + 2.57 | 0.574 |
| NOD2 = 0.83 * TNFRSF9 + 2.35 | 0.557 |
| NOD2 = 0.71 * SNAI3 + 3.34 | 0.547 |
| NOD2 = 0.99 * NLRP3 + 0.82 | 0.543 |
| NOD2 = 0.95 * TLR2 + 0.93 | 0.535 |
| NOD2 = 0.78 * AQP9 + 1.91 | 0.522 |
| NOTCH1 = 0.73 * ANAPC2 + 2.50 | 0.559 |
| NOTCH1 = 0.71 * SPC25 + 4.07 | 0.478 |
| NOTCH1 = 0.66 * GSN + 2.45 | 0.463 |
| NOTCH4 = 1.28 * DLL4 − 2.43 | 0.677 |
| NOTCH4 = 1.04 * CD34 − 1.90 | 0.642 |
| NOTCH4 = 1.19 * KDR − 3.44 | 0.586 |
| NOTCH4 = 1.10 * RAMP2 − 2.46 | 0.570 |
| NOTCH4 = 1.12 * HEYL − 2.44 | 0.566 |
| NOTCH4 = 0.42 * ER_109 + 5.38 | 0.559 |
| NR6A1 = 0.63 * OLIG2 + 2.99 | 0.649 |
| NR6A1 = 0.59 * MADCAM1 + 3.23 | 0.642 |
| NR6A1 = 0.90 * ATP6V1G2 + 0.43 | 0.641 |
| NR6A1 = 0.64 * WNT7A + 2.87 | 0.638 |
| NR6A1 = 1.06 * MUTYH − 0.41 | 0.638 |
| NR6A1 = 0.67 * PARP3 + 2.98 | 0.636 |
| NRG1 = 1.13 * FGF1 − 0.68 | 0.651 |
| NRG1 = 0.86 * MAGEL2 + 1.84 | 0.647 |
| NRG1 = 1.30 * NOX4 − 3.61 | 0.644 |
| NRG1 = 0.82 * FGF16 + 2.26 | 0.626 |
| NRG1 = 0.90 * FAM133A + 1.32 | 0.626 |
| NRG1 = 1.20 * ABCB4 − 1.26 | 0.625 |
| NSD1 = 0.93 * FBXW11 − 0.17 | 0.570 |
| NSD1 = 1.06 * PFDN1 − 1.50 | 0.468 |
| NSD1 = 1.10 * MAML1 − 0.33 | 0.443 |
| NTHL1 = 1.22 * PELP1 − 2.33 | 0.526 |
| NTHL1 = 1.60 * TSC2 − 8.13 | 0.463 |
| NTHL1 = −1.15 * SLC2A3 + 17.07 | −0.459 |
| NTRK1 = 0.80 * HAND1 + 1.26 | 0.830 |
| NTRK1 = 0.87 * SLC3A1 + 0.04 | 0.815 |
| NTRK1 = 0.89 * FGF8 + 0.40 | 0.814 |
| NTRK1 = 0.85 * CHGA + 1.16 | 0.812 |
| NTRK1 = 0.77 * HNF1B + 1.42 | 0.810 |
| NTRK1 = 0.80 * GATA1 + 1.04 | 0.808 |
| NTRK1 = 0.84 * WNT7A + 0.99 | 0.801 |
| NTRK1 = 0.93 * NFE2L2 + 0.68 | 0.801 |
| NTRK1 = 0.97 * PTPN5 − 0.63 | 0.801 |
| NTRK1 = 0.79 * ADRA1D + 1.67 | 0.800 |
| NUMBL = 1.13 * PELP1 − 1.81 | 0.481 |
| NUMBL = −1.21 * CASP4 + 17.77 | −0.467 |
| NUMBL = 0.37 * SLC22A6 + 6.23 | 0.448 |
| ORM2 = 0.91 * ORM1 + 0.93 | 0.776 |
| ORM2 = 0.88 * CASP14 + 0.78 | 0.579 |
| ORM2 = 1.16 * GATA5 + 0.30 | 0.571 |
| ORM2 = 1.40 * MIXL1 − 1.98 | 0.564 |
| ORM2 = 1.43 * ABCC6 − 1.63 | 0.557 |
| ORM2 = 1.25 * ESRRB − 0.21 | 0.556 |
| P4HB = 1.47 * PRKAR1A − 4.74 | 0.550 |
| P4HB = 1.30 * PPIB − 3.88 | 0.541 |
| P4HB = −0.60 * PAX5 + 15.62 | −0.536 |
| P4HB = 1.07 * TK1 + 2.70 | 0.514 |
| P4HB = −0.63 * TSHR + 16.41 | −0.513 |
| P4HB = 0.85 * SLC16A3 + 3.79 | 0.512 |
| PAG1 = 0.89 * SLA + 0.12 | 0.645 |
| PAG1 = 0.58 * CCR2 + 3.90 | 0.642 |
| PAG1 = 0.57 * IL2RG + 3.12 | 0.633 |
| PAG1 = 0.63 * IRF8 + 2.21 | 0.632 |
| PAG1 = 0.65 * CXCR6 + 2.97 | 0.626 |
| PAG1 = 0.68 * PRKCB + 2.69 | 0.624 |
| PARP2 = 0.86 * APEX1 − 0.57 | 0.474 |
| PARP2 = 1.01 * BCL2L2 − 0.57 | 0.435 |
| PARP2 = 0.75 * PLK4 + 2.20 | 0.332 |
| PAX6 = −4.61 * NCK2 + 49.27 | −0.435 |
| PAX6 = 2.11 * ZIC2 − 8.32 | 0.392 |
| PAX6 = 1.23 * ER_028 + 1.56 | 0.392 |
| PCOLCE = 1.06 * PDGFRB − 0.19 | 0.771 |
| PCOLCE = 0.73 * COL5A2 + 1.70 | 0.750 |
| PCOLCE = 0.64 * COL3A1 + 0.27 | 0.740 |
| PCOLCE = 0.75 * COL5A1 + 1.11 | 0.729 |
| PCOLCE = 0.73 * MMP2 + 0.70 | 0.716 |
| PCOLCE = 0.93 * THY1 + 0.68 | 0.710 |
| PCOLCE = 0.65 * COL1A2 + 1.33 | 0.706 |
| PCOLCE = 0.98 * TIMP2 − 1.83 | 0.701 |
| PDCD1LG2 = 1.07 * CYBB − 4.19 | 0.802 |
| PDCD1LG2 = 1.60 * CD86 − 6.38 | 0.724 |
| PDCD1LG2 = 1.14 * CD74 − 10.38 | 0.708 |
| PDCD1LG2 = 1.19 * CTSS − 5.34 | 0.705 |
| PDCD1LG2 = 1.12 * FCGR1A − 2.47 | 0.701 |
| PDCD1LG2 = 1.22 * FGL2 − 5.55 | 0.696 |
| PDGFB = 1.07 * DLC1 + 0.78 | 0.630 |
| PDGFB = 0.74 * CTGF + 0.71 | 0.608 |
| PDGFB = 1.22 * PDGFRB − 2.39 | 0.568 |
| PDGFB = 0.92 * BMP8A + 2.53 | 0.566 |
| PDGFB = 1.28 * IGFBP7 − 5.81 | 0.526 |
| PDGFB = 1.13 * TIMP2 − 4.29 | 0.515 |
| PFKFB3 = 0.46 * ANGPTL4 + 5.62 | 0.516 |
| PFKFB3 = 0.64 * ADM + 3.25 | 0.482 |
| PFKFB3 = 0.78 * PFKFB4 + 3.59 | 0.448 |
| PHB = 0.91 * DNAJC8 + 2.09 | 0.548 |
| PHB = 0.80 * AURKA + 3.44 | 0.483 |
| PHB = 1.10 * ATP5G1 − 1.36 | 0.479 |
| PIK3CA = 0.52 * LINC00886 + 5.37 | 0.489 |
| PIK3CA = 0.95 * ERCC4 + 1.82 | 0.477 |
| PIK3CA = 0.85 * KATNBL1 + 2.27 | 0.474 |
| PIM3 = 0.74 * MIF + 0.45 | 0.535 |
| PIM3 = 0.81 * CCT4 + 1.23 | 0.518 |
| PIM3 = 0.62 * XRCC5 + 5.30 | 0.494 |
| PLA2G10 = 0.90 * PLA2G3 + 2.38 | 0.900 |
| PLA2G10 = 0.88 * WNT1 + 2.35 | 0.857 |
| PLA2G10 = 0.85 * MYOD1 + 3.04 | 0.851 |
| PLA2G10 = 0.92 * CEACAM3 + 2.12 | 0.845 |
| PLA2G10 = 1.15 * TIE1 + 0.12 | 0.839 |
| PLA2G10 = 0.99 * IL4 + 1.59 | 0.835 |
| PLA2G10 = 0.93 * CMTM2 + 2.59 | 0.834 |
| PLA2G10 = 0.90 * LEP + 2.43 | 0.830 |
| PLA2G10 = 0.84 * CAMK2B + 3.01 | 0.827 |
| PLA2G10 = 1.01 * CECR6 + 1.31 | 0.824 |
| PLA2G4A = 0.79 * PTGS2 + 2.80 | 0.577 |
| PLA2G4A = 1.42 * TLR5 − 3.07 | 0.361 |
| PLA2G4A = −1.99 * KDM5C + 28.92 | −0.360 |
| PLAT = 1.22 * PDGFRB − 2.59 | 0.605 |
| PLAT = 0.83 * COL5A2 − 0.42 | 0.600 |
| PLAT = 1.12 * TIMP2 − 4.49 | 0.591 |
| PLAT = 0.75 * COL1A2 − 0.84 | 0.587 |
| PLAT = 0.86 * COL5A1 − 1.10 | 0.584 |
| PLAT = 1.08 * THY1 − 1.68 | 0.584 |
| PLCB1 = 0.51 * WIF1 + 5.24 | 0.491 |
| PLCB1 = 1.33 * CRLS1 − 1.48 | 0.464 |
| PLCB1 = 0.88 * IRS1 + 1.48 | 0.458 |
| PLCG1 = 0.81 * KMT2D + 1.07 | 0.392 |
| PLCG1 = 0.87 * PNKP + 0.99 | 0.389 |
| PLCG1 = 0.58 * ULK1 + 3.53 | 0.386 |
| PLCG2 = 0.53 * CD38 + 4.47 | 0.632 |
| PLCG2 = 0.63 * PIM2 + 2.73 | 0.594 |
| PLCG2 = 0.42 * IRF4 + 5.33 | 0.575 |
| PLCG2 = 0.51 * CD79A + 4.58 | 0.563 |
| PLCG2 = 0.93 * CCR1 + 0.37 | 0.555 |
| PLCG2 = 0.82 * IL10RA + 0.87 | 0.554 |
| PLK4 = 0.97 * MAD2L1 + 0.03 | 0.660 |
| PLK4 = 0.86 * CCNA2 + 1.18 | 0.618 |
| PLK4 = 1.13 * SMC4 − 1.07 | 0.575 |
| PLK4 = 0.81 * BUB1B + 2.93 | 0.567 |
| PLK4 = 0.92 * NEIL3 +1.83 | 0.550 |
| PLK4 = 0.85 * HJURP + 1.96 | 0.549 |
| PMEPA1 = 0.83 * FN1 − 2.86 | 0.650 |
| PMEPA1 = 0.65 * COL11A1 + 2.73 | 0.610 |
| PMEPA1 = 0.82 * COL1A2 − 1.21 | 0.609 |
| PMEPA1 = 0.96 * INHBA + 0.10 | 0.601 |
| PMEPA1 = 1.50 * SERPINH1 − 8.40 | 0.597 |
| PMEPA1 = 0.58 * EDIL3 + 4.69 | 0.591 |
| PML = 0.44 * ITGB7 + 6.68 | 0.583 |
| PML = 0.86 * PRKACA + 1.99 | 0.551 |
| PML = 0.56 * CD47 + 5.11 | 0.534 |
| PML = 0.59 * IFI27 + 3.59 | 0.532 |
| PML = 0.78 * TNFAIP2 + 3.03 | 0.504 |
| PPARGC1A = 1.02 * MSTN − 0.58 | 0.551 |
| PPARGC1A = 0.69 * COL11A2 + 2.66 | 0.535 |
| PPARGC1A = 1.06 * NGF + 0.22 | 0.508 |
| PPARGC1A = 1.25 * RAG1 − 2.17 | 0.507 |
| PPARGC1A = 0.94 * NCAM1 + 0.88 | 0.505 |
| PPARGC1A = 1.25 * RBPMS2 − 1.66 | 0.504 |
| PPID = 1.03 * MAT2A − 2.92 | 0.509 |
| PPID = 0.63 * CCT6A + 1.83 | 0.503 |
| PPID = 1.01 * SETD2 − 1.61 | 0.485 |
| PPP2CA = 0.83 * VDAC1 + 1.78 | 0.660 |
| PPP2CA = 0.57 * VAMP8 + 5.22 | 0.615 |
| PPP2CA = 0.61 * HSPA4 + 4.55 | 0.596 |
| PPP2CA = 0.81 * HSPA8 + 0.27 | 0.554 |
| PPP2CA = −0.34 * HHAT + 13.18 | −0.528 |
| PPP2CA = 0.86 * PRKAG1 + 3.14 | 0.526 |
| PPP2CB = 0.61 * PDLIM7 + 4.32 | 0.481 |
| PPP2CB = 0.71 * SERPINH1 + 1.78 | 0.464 |
| PPP2CB = 0.39 * COL1A2 + 5.18 | 0.442 |
| PRAME = 1.06 * HOXB13 + 1.69 | 0.327 |
| PRC1 = 1.01 * BLM + 1.20 | 0.626 |
| PRC1 = 0.99 * DLGAP5 + 0.49 | 0.620 |
| PRC1 = 0.93 * CDC20 − 0.81 | 0.592 |
| PRC1 = 1.04 * HJURP + 0.17 | 0.564 |
| PRC1 = 1.39 * RACGAP1 − 4.52 | 0.550 |
| PRC1 = 0.91 * GTSE1 + 1.21 | 0.543 |
| PRDM1 = 0.88 * TLR8 + 3.05 | 0.669 |
| PRDM1 = 1.14 * SLA − 1.79 | 0.668 |
| PRDM1 = 0.65 * TNFRSF17 + 3.81 | 0.659 |
| PRDM1 = 0.98 * CASP10 + 0.64 | 0.646 |
| PRDM1 = 1.27 * TLR4 − 2.23 | 0.625 |
| PRDM1 = 1.18 * SYK − 1.81 | 0.618 |
| PRKAA2 = 1.01 * ABCG2 + 0.19 | 0.513 |
| PRKAA2 = 0.80 * MSTN + 1.40 | 0.510 |
| PRKAA2 = 1.04 * BCL2L10 − 0.08 | 0.509 |
| PRKAA2 = 1.07 * TNFSF13B − 0.24 | 0.504 |
| PRKAA2 = 0.91 * BMP8B + 1.37 | 0.501 |
| PRKAG1 = −0.43 * NPM1 + 11.69 | −0.640 |
| PRKAG1 = −0.52 * TGFB1 + 12.88 | −0.631 |
| PRKAG1 = 0.74 * COX7B + 0.91 | 0.629 |
| PRKAG1 = −0.30 * HSPA2 + 10.83 | −0.626 |
| PRKAG1 = −0.35 * RPA3 + 11.23 | −0.623 |
| PRKAG1 = −0.36 * BCL6 + 11.44 | −0.620 |
| PRKCE = 0.84 * MSH2 + 0.84 | 0.416 |
| PRKCE = 0.87 * RPS6KA5 + 1.73 | 0.413 |
| PRKCE = 0.75 * KAT5 + 3.10 | 0.402 |
| PRMT6 = 0.67 * CHEK1 + 2.37 | 0.713 |
| PRMT6 = 0.49 * FGF21 + 3.77 | 0.701 |
| PRMT6 = 0.48 * CRYAA + 3.76 | 0.689 |
| PRMT6 = 0.52 * LTA + 3.76 | 0.679 |
| PRMT6 = 0.66 * TNFRSF10C + 1.73 | 0.678 |
| PRMT6 = 0.46 * HSPA2 + 4.17 | 0.678 |
| PROM1 = 1.19 * VTCN1 − 2.00 | 0.457 |
| PROM1 = 1.26 * EFNA5 − 1.61 | 0.453 |
| PROM1 = 1.67 * ITGB8 − 5.83 | 0.432 |
| PRR15L = 0.86 * MUC1 − 1.15 | 0.532 |
| PRR15L = 1.71 * CREB3L4 − 7.08 | 0.497 |
| PRR15L = 0.74 * CCL28 + 2.28 | 0.492 |
| PSIP1 = −0.91 * LOXL1 + 17.64 | −0.535 |
| PSIP1 = 0.99 * MELK + 0.65 | 0.504 |
| PSIP1 = −1.11 * PDGFRB + 19.81 | −0.502 |
| PSMD2 = 1.04 * EIF4G1 − 1.17 | 0.804 |
| PSMD2 = −0.65 * TGFB1 + 15.50 | −0.557 |
| PSMD2 = −0.50 * CCDC103 + 13.70 | −0.551 |
| PSMD2 = 1.05 * CALR − 2.39 | 0.528 |
| PSMD2 = −0.64 * S1PR1 + 15.35 | −0.522 |
| PSMD2 = −0.45 * RND2 + 13.75 | −0.522 |
| PTCHD1 = 0.95 * NCAM1 + 0.89 | 0.466 |
| PTCHD1 = 1.13 * FGF13 − 2.17 | 0.450 |
| PTCHD1 = 0.91 * ALK + 0.82 | 0.446 |
| PTGR1 = 1.23 * TOP3A − 2.47 | 0.492 |
| PTGR1 = 0.90 * PRKACA − 0.50 | 0.483 |
| PTGR1 = 1.12 * VEGFB − 3.19 | 0.479 |
| PTP4A1 = 0.73 * TBP + 4.42 | 0.472 |
| PTP4A1 = 1.21 * PPIB − 6.12 | 0.447 |
| PTP4A1 = −1.13 * HERC3 + 18.91 | −0.403 |
| PTPN11 = 0.27 * SOX2 + 8.00 | 0.554 |
| PTPN11 = 0.85 * TXNRD1 + 2.08 | 0.515 |
| PTPN11 = 0.72 * ATF4 + 2.52 | 0.509 |
| PTPN11 = 1.04 * TDG − 0.69 | 0.500 |
| PTPRC = 0.76 * PPP3R2 + 2.27 | 0.658 |
| PTPRC = 0.82 * INS − 0.01 | 0.648 |
| PTPRC = 0.62 * CD19 + 3.91 | 0.623 |
| PTPRC = 0.54 * LAMB4 + 4.07 | 0.622 |
| PTPRC = 0.72 * HNF1A + 1.34 | 0.617 |
| PTPRC = 1.29 * MENG − 2.49 | 0.604 |
| PTTG1 = 0.98 * DNAJB14 − 1.40 | 0.782 |
| PTTG1 = 0.97 * EGLN1 − 0.38 | 0.735 |
| PTTG1 = 1.17 * FANCC − 2.04 | 0.726 |
| PTTG1 = 0.81 * HSPA9 − 1.51 | 0.720 |
| PTTG1 = 1.03 * TRIB1 − 2.51 | 0.682 |
| PTTG1 = 1.22 * SLC26A2 − 1.83 | 0.678 |
| PYCR1 = 1.22 * GAA − 2.91 | 0.474 |
| PYCR1 = 1.16 * P4HB − 5.71 | 0.470 |
| PYCR1 = −1.84 * RBPJ + 25.70 | −0.468 |
| QSOX2 = 1.01 * TTF1 − 0.15 | 0.486 |
| QSOX2 = 0.62 * PTCH1 + 2.88 | 0.425 |
| QSOX2 = 0.71 * IL6R + 1.38 | 0.413 |
| RAB6B = 0.80 * ALK + 1.91 | 0.781 |
| RAB6B = 0.82 * SLC7A9 + 1.83 | 0.768 |
| RAB6B = 0.81 * CRP + 1.84 | 0.767 |
| RAB6B = 0.77 * CCL8 + 2.42 | 0.763 |
| RAB6B = 0.92 * POU5F1 − 0.65 | 0.761 |
| RAB6B = 0.73 * MAGEA11 + 2.18 | 0.761 |
| RAB6B = 0.77 * THPO + 2.51 | 0.759 |
| RAB6B = 0.78 * S100A8 + 2.02 | 0.758 |
| RAB6B = 0.63 * CYP1A2 + 3.50 | 0.757 |
| RAB6B = 0.82 * APCS + 1.63 | 0.755 |
| RAC3 = 1.20 * P4HB − 5.73 | 0.464 |
| RAC3 = 1.03 * PYCR1 + 0.19 | 0.455 |
| RAC3 = 0.93 * FASN − 0.67 | 0.431 |
| RAD51C = 0.87 * AKAP1 − 1.34 | 0.490 |
| RAD51C = −0.80 * CD14 + 15.16 | −0.402 |
| RAD51C = 0.88 * NME1 − 1.78 | 0.375 |
| RAD9A = 0.87 * POLD4 + 2.32 | 0.558 |
| RAD9A = 0.98 * MKNK1 + 0.91 | 0.553 |
| RAD9A = 0.76 * GPR180 + 2.99 | 0.550 |
| RAD9A = 0.77 * BLM + 2.50 | 0.535 |
| RAD9A = 0.80 * FES + 2.38 | 0.522 |
| RAD9A = 0.47 * SLC7A9 + 5.09 | 0.517 |
| RARB = 0.72 * TBX3 + 3.82 | 0.365 |
| RARB = 1.12 * MACC1 − 3.05 | 0.347 |
| RARB = −0.94 * ADORA2B + 13.69 | −0.345 |
| RASSF1 = 0.41 * IL10 + 5.25 | 0.503 |
| RASSF1 = 1.09 * GNL3 − 3.74 | 0.421 |
| RASSF1 = 0.28 * ER_160 + 6.60 | 0.419 |
| RB1 = 1.50 * RBL2 − 5.32 | 0.497 |
| RB1 = −1.37 * DNAJC8 + 21.64 | −0.471 |
| RB1 = −0.95 * FAM64A + 16.53 | −0.466 |
| RBP1 = 1.71 * LTBP1 − 7.72 | 0.378 |
| RBP1 = −1.87 * CMKLR1 + 22.31 | −0.345 |
| RBP1 = 1.46 * ITGA2 − 1.79 | 0.340 |
| RELN = 1.55 * ABCA9 − 5.50 | 0.652 |
| RELN = 1.08 * CCL14 − 2.29 | 0.629 |
| RELN = 1.51 * HGF − 3.70 | 0.607 |
| RELN = 1.62 * TSPAN7 − 6.18 | 0.597 |
| RELN = 2.02 * SLIT2 − 11.25 | 0.578 |
| RELN = 1.32 * IL33 − 4.98 | 0.573 |
| RIPK3 = 0.56 * CD27 + 2.98 | 0.669 |
| RIPK3 = 0.69 * CD3D + 1.47 | 0.655 |
| RIPK3 = 1.03 * TNFRSF1B − 2.30 | 0.644 |
| RIPK3 = 1.01 * CMKLR1 + 0.17 | 0.643 |
| RIPK3 = 1.14 * FLT3LG − 2.05 | 0.639 |
| RIPK3 = 0.46 * IRF4 + 3.90 | 0.635 |
| RPL13 = 1.01 * PRKAB1 − 0.59 | 0.590 |
| RPL13 = 0.45 * IFT52 + 3.77 | 0.540 |
| RPL13 = 0.62 * SMAD9 + 3.13 | 0.537 |
| RPL13 = 1.34 * SMUG1 − 2.61 | 0.529 |
| RPL13 = 0.43 * MPO + 5.16 | 0.501 |
| RPL6 = 1.44 * SLC25A3 − 6.13 | 0.610 |
| RPL6 = 0.97 * EEF1G − 2.27 | 0.585 |
| RPL6 = 1.00 * RPS7 − 2.04 | 0.576 |
| RPL6 = 1.15 * NAP1L1 − 3.22 | 0.558 |
| RPL6 = 1.66 * HNRNPA1 − 7.28 | 0.553 |
| RPL6 = 1.38 * TDG − 2.36 | 0.549 |
| RUNX1 = 0.91 * ACTB − 1.85 | 0.892 |
| RUNX1 = 0.95 * HSPA9 + 0.95 | 0.866 |
| RUNX1 = 1.50 * MMS19 − 3.94 | 0.833 |
| RUNX1 = 1.10 * TRIB1 + 0.60 | 0.808 |
| RUNX1 = 1.06 * DNAJB14 + 1.77 | 0.801 |
| RUNX1 = 1.90 * YY1 − 7.62 | 0.795 |
| RUNX1 = 1.43 * TICAM1 − 0.68 | 0.794 |
| RUNX1 = 1.42 * WASL − 0.47 | 0.793 |
| RUNX1 = 1.30 * LAMA5 − 1.86 | 0.792 |
| RUNX1 = 1.56 * DNAJC7 − 4.01 | 0.790 |
| S100A6 = 1.15 * S100A4 + 0.63 | 0.597 |
| S100A6 = 0.60 * KRT17 + 7.80 | 0.509 |
| S100A6 = 1.16 * ANXA1 + 0.92 | 0.505 |
| SCUBE2 = 1.11 * HOXA9 − 1.22 | 0.643 |
| SCUBE2 = 1.21 * GATA2 − 2.14 | 0.643 |
| SCUBE2 = 1.38 * GALNT5 − 2.96 | 0.642 |
| SCUBE2 = 1.14 * AR − 1.48 | 0.631 |
| SCUBE2 = 1.32 * CX3CR1 − 2.88 | 0.629 |
| SCUBE2 = 1.38 * GHR − 3.76 | 0.627 |
| SELE = 1.08 * ANGPTL1 − 0.62 | 0.595 |
| SELE = 0.85 * SLCO1B3 + 2.18 | 0.585 |
| SELE = 1.07 * KLRG1 − 1.10 | 0.567 |
| SELE = 1.45 * HHEX − 3.89 | 0.563 |
| SELE = 1.03 * CD80 + 0.60 | 0.563 |
| SELE = 1.04 * F8 + 0.20 | 0.562 |
| SERPINB2 = 0.95 * KCNIP1 + 0.26 | 0.712 |
| SERPINB2 = 0.90 * MBL2 + 1.14 | 0.712 |
| SERPINB2 = 0.94 * NODAL + 0.26 | 0.708 |
| SERPINB2 = 1.11 * CXCR2 − 0.91 | 0.703 |
| SERPINB2 = 0.92 * NPPB + 0.64 | 0.694 |
| SERPINB2 = 0.99 * CRP − 0.48 | 0.690 |
| SERPINF1 = 0.73 * MMP2 + 2.12 | 0.716 |
| SERPINF1 = 0.77 * FBN1 + 3.04 | 0.700 |
| SERPINF1 = 0.57 * SFRP2 + 4.39 | 0.685 |
| SERPINF1 = 0.62 * SFRP4 + 5.21 | 0.677 |
| SERPINF1 = 0.63 * COL1A1 + 1.50 | 0.668 |
| SERPINF1 = 0.66 * COL1A2 + 2.76 | 0.665 |
| SETD2 = 0.85 * HDAC8 + 3.40 | 0.712 |
| SETD2 = 0.63 * CCT6A + 3.41 | 0.709 |
| SETD2 = 1.02 * MAT2A − 1.30 | 0.685 |
| SFRP2 = 1.16 * COL1A2 − 2.90 | 0.822 |
| SFRP2 = 1.11 * COL1A1 − 5.11 | 0.814 |
| SFRP2 = 1.13 * COL3A1 − 4.78 | 0.807 |
| SFRP2 = 1.29 * MMP2 − 4.01 | 0.798 |
| SFRP2 = 1.29 * COL5A2 − 2.24 | 0.785 |
| SFRP2 = 1.36 * FBN1 − 2.39 | 0.782 |
| SFRP2 = 1.44 * SPARC − 6.87 | 0.775 |
| SFRP2 = 1.33 * COL5A1 − 3.30 | 0.724 |
| SFRP4 = 0.91 * SFRP2 − 1.38 | 0.693 |
| SFRP4 = 1.61 * SERPINF1 − 8.37 | 0.677 |
| SFRP4 = 1.24 * FBN1 − 3.48 | 0.663 |
| SFRP4 = 1.53 * RASGRF2 − 1.82 | 0.624 |
| SFRP4 = 2.09 * ZEB1 − 9.00 | 0.621 |
| SFRP4 = 1.31 * SPARC − 7.60 | 0.618 |
| SHC2 = 1.34 * FLNC − 1.92 | 0.513 |
| SHC2 = 1.28 * CAMK2N1 − 4.02 | 0.477 |
| SHC2 = 1.70 * ETV1 − 4.94 | 0.467 |
| SLAMF7 = 0.93 * IRF4 + 1.70 | 0.882 |
| SLAMF7 = 1.16 * CD38 − 0.19 | 0.862 |
| SLAMF7 = 1.14 * CD27 − 0.14 | 0.849 |
| SLAMF7 = 1.80 * IL10RA − 8.22 | 0.848 |
| SLAMF7 = 1.39 * PIM2 − 4.04 | 0.843 |
| SLAMF7 = 1.48 * IL2RG − 4.63 | 0.843 |
| SLAMF7 = 1.77 * FGL2 − 9.67 | 0.824 |
| SLAMF7 = 1.05 * CXCR3 + 1.24 | 0.809 |
| SLAMF7 = 1.68 * CCR5 − 4.88 | 0.793 |
| SLAMF7 = 1.72 * APOL3 − 7.35 | 0.790 |
| SLC11A1 = 0.72 * FGF8 + 2.75 | 0.693 |
| SLC11A1 = 0.89 * CCRL2 + 1.79 | 0.686 |
| SLC11A1 = 0.85 * TNFSF9 + 1.73 | 0.685 |
| SLC11A1 = 0.65 * KRT13 + 3.48 | 0.683 |
| SLC11A1 = 0.73 * NPPB + 2.70 | 0.680 |
| SLC11A1 = 0.60 * T + 3.57 | 0.675 |
| SLC16A1 = 1.47 * NCL − 9.31 | 0.465 |
| SLC16A1 = 0.95 * TOP2A − 0.31 | 0.448 |
| SLC16A1 = − 0.63 * MLPH + 13.59 | −0.448 |
| SLC16A2 = 0.77 * MPL + 1.77 | 0.603 |
| SLC16A2 = 0.75 * CCL26 + 1.95 | 0.602 |
| SLC16A2 = 0.82 * IL13RA2 + 1.21 | 0.594 |
| SLC16A2 = 0.82 * F8 + 0.75 | 0.593 |
| SLC16A2 = 0.97 * TSC22D1 − 0.12 | 0.592 |
| SLC16A2 = 0.52 * CCL1 + 4.29 | 0.592 |
| SLC25A13 = −0.62 * TNF + 13.95 | −0.386 |
| SLC25A13 = 0.82 * HSPE1 − 0.11 | 0.345 |
| SLC25A13 = 1.09 * SWAP70 − 0.60 | 0.342 |
| SLC45A3 = 0.95 * KIF14 + 1.34 | 0.737 |
| SLC45A3 = 0.93 * PMS1 + 1.11 | 0.731 |
| SLC45A3 = 0.79 * CECR6 + 2.51 | 0.706 |
| SLC45A3 = 0.94 * NOS3 + 0.74 | 0.696 |
| SLC45A3 = 1.06 * MCM7 + 0.33 | 0.686 |
| SLC45A3 = 1.18 * CYCS + 0.16 | 0.685 |
| SLIT2 = 0.81 * TSPAN7 + 2.48 | 0.757 |
| SLIT2 = 0.72 * DKK2 + 3.67 | 0.720 |
| SLIT2 = 0.99 * RUNX1T1 + 0.18 | 0.700 |
| SLIT2 = 0.59 * FGF16 + 4.91 | 0.676 |
| SLIT2 = 0.65 * MS4A1 + 4.03 | 0.670 |
| SLIT2 = 0.70 * CX3CR1 + 3.41 | 0.670 |
| SMAD2 = 0.83 * PIAS2 + 2.99 | 0.544 |
| SMAD2 = 0.95 * PIK3C3 + 1.74 | 0.484 |
| SMAD2 = 0.71 * SLC39A6 + 3.18 | 0.468 |
| SMC1A = 1.04 * KDM5C − 0.13 | 0.577 |
| SMC1A = 0.52 * TOP2A + 5.48 | 0.473 |
| SMC1A = 0.60 * CKS2 + 4.37 | 0.472 |
| SMC4 = 0.89 * PLK4 + 0.95 | 0.575 |
| SMC4 = 0.78 * EZH2 + 1.30 | 0.562 |
| SMC4 = 0.87 * MAD2L1 + 0.92 | 0.559 |
| SMC4 = 0.77 * CCNA2 + 2.00 | 0.543 |
| SMC4 = 0.80 * PTTG2 + 1.18 | 0.542 |
| SMC4 = 0.81 * ECT2 + 0.94 | 0.542 |
| SNCA = 0.61 * SLC2A2 + 5.11 | 0.485 |
| SNCA = 0.47 * ER_109 + 5.40 | 0.476 |
| SNCA = 0.52 * CCL16 + 5.14 | 0.457 |
| SOCS4 = 0.81 * DNAJC8 + 0.70 | 0.459 |
| SOCS4 = 0.30 * MAGEB2 + 6.99 | 0.445 |
| SOCS4 = 0.62 * HDAC8 + 3.33 | 0.432 |
| SORT1 = −0.69 * LAG3 + 15.22 | −0.514 |
| SORT1 = 0.63 * VTCN1 + 3.32 | 0.504 |
| SORT1 = −0.67 * OASL + 15.01 | −0.488 |
| SPARC = 0.77 * COL1A1 + 1.22 | 0.900 |
| SPARC = 0.81 * COL1A2 + 2.76 | 0.893 |
| SPARC = 0.79 * COL3A1 + 1.45 | 0.884 |
| SPARC = 0.89 * COL5A2 + 3.21 | 0.860 |
| SPARC = 0.92 * COL5A1 + 2.48 | 0.802 |
| SPARC = 0.91 * LOX + 4.47 | 0.798 |
| SPARC = 0.95 * FBN1 + 3.10 | 0.793 |
| SPARC = 0.69 * SFRP2 + 4.76 | 0.775 |
| SPARC = 0.58 * EDIL3 + 8.52 | 0.744 |
| SPARC = 0.90 * MMP2 + 1.98 | 0.743 |
| SPDEF = 1.06 * FOXA1 − 0.66 | 0.678 |
| SPDEF = 2.76 * CREB3L4 − 17.61 | 0.586 |
| SPDEF = 2.74 * ZNF552 − 15.65 | 0.554 |
| SPDEF = 2.08 * FASN − 15.29 | 0.522 |
| SPINK1 = 1.38 * FGF1 − 3.53 | 0.648 |
| SPINK1 = 1.60 * NOX4 − 7.17 | 0.638 |
| SPINK1 = 1.33 * KCND2 − 2.94 | 0.602 |
| SPINK1 = 1.05 * MAGEL2 − 0.45 | 0.590 |
| SPINK1 = 1.10 * CA12 − 1.68 | 0.589 |
| SPINK1 = 1.47 * ABCB4 − 4.24 | 0.588 |
| SPOP = −0.54 * CDK16 + 15.44 | −0.516 |
| SPOP = 0.30 * STAB1 + 7.83 | 0.486 |
| SPOP = 0.54 * FAM105A + 5.45 | 0.482 |
| SPRY2 = 1.21 * DNAJB14 − 2.13 | 0.617 |
| SPRY2 = 1.08 * EGLN1 + 0.00 | 0.614 |
| SPRY2 = 0.78 * HSPA6 + 3.48 | 0.569 |
| SPRY2 = 1.28 * DISP1 − 1.91 | 0.554 |
| SPRY2 = 0.81 * TNXB + 1.85 | 0.536 |
| SPRY2 = 0.58 * FOXD3 + 4.64 | 0.532 |
| SPRY4 = 0.82 * DUSP6 + 1.37 | 0.569 |
| SPRY4 = 0.90 * ETV1 + 1.53 | 0.532 |
| SPRY4 = 0.55 * ITGB3 + 5.42 | 0.531 |
| SPRY4 = 0.86 * STX1A + 1.80 | 0.530 |
| SPRY4 = 0.70 * FLT4 + 3.84 | 0.522 |
| SPRY4 = 0.95 * DLL4 + 1.94 | 0.504 |
| SRF = 0.75 * FRS3 + 4.90 | 0.490 |
| SRF = 0.64 * CCT4 + 2.73 | 0.483 |
| SRF = 1.22 * ABCC10 − 1.13 | 0.466 |
| SRM = 1.05 * KDM1A + 0.76 | 0.478 |
| SRM = 1.13 * DNAJC11 + 1.01 | 0.471 |
| SRM = 1.38 * MTOR − 2.15 | 0.462 |
| STAT1 = 0.87 * GBP1 + 2.41 | 0.854 |
| STAT1 = 0.93 * TAP1 + 2.69 | 0.833 |
| STAT1 = 0.74 * CCL5 + 4.86 | 0.793 |
| STAT1 = 0.64 * CXCL10 + 5.44 | 0.775 |
| STAT1 = 1.05 * CTSS + 0.95 | 0.767 |
| STAT1 = 1.05 * APOL3 + 2.19 | 0.761 |
| STAT1 = 0.55 * CXCL9 + 6.11 | 0.753 |
| STAT1 = 1.09 * FGL2 + 0.77 | 0.752 |
| STAT1 = 1.01 * CD74 − 3.51 | 0.746 |
| STAT1 = 1.05 * HLA_A − 2.93 | 0.743 |
| STEAP4 = 0.86 * ZBTB16 + 2.29 | 0.619 |
| STEAP4 = 1.20 * HGF + 0.55 | 0.576 |
| STEAP4 = 1.13 * FMO5 + 0.28 | 0.572 |
| STEAP4 = 0.76 * LRG1 + 3.35 | 0.571 |
| STEAP4 = 0.96 * ACKR1 + 1.24 | 0.547 |
| STEAP4 = 0.85 * CCL14 + 1.72 | 0.546 |
| STK3 = 0.75 * RAD21 + 1.53 | 0.627 |
| STK3 = 0.96 * PTK2 − 0.28 | 0.603 |
| STK3 = 0.88 * PTDSS1 + 1.17 | 0.535 |
| STK3 = 0.87 * HSF1 + 1.34 | 0.503 |
| STK39 = 0.45 * UTY + 6.63 | 0.341 |
| STK39 = 0.49 * ARNT2 + 4.27 | 0.335 |
| STK39 = 0.58 * SLC22A3 + 5.10 | 0.331 |
| STX1A = 0.49 * FOXE1 + 4.49 | 0.668 |
| STX1A = 0.98 * ATP7A + 0.85 | 0.651 |
| STX1A = 0.56 * ADRA2B + 4.29 | 0.631 |
| STX1A = 0.52 * CCL24 + 4.39 | 0.615 |
| STX1A = 0.79 * RASA4 + 0.95 | 0.613 |
| STX1A = 0.77 * DTX1 + 2.44 | 0.612 |
| TADA3 = 0.99 * MEN1 − 0.00 | 0.609 |
| TADA3 = 0.90 * ELK1 + 0.91 | 0.602 |
| TADA3 = 0.43 * SLC7A5 + 5.66 | 0.592 |
| TADA3 = 0.52 * ABCC4 + 5.17 | 0.582 |
| TADA3 = 0.47 * MMS19 + 4.83 | 0.581 |
| TADA3 = 0.53 * YY1 + 4.24 | 0.573 |
| TAP1 = 1.08 * STAT1 − 2.89 | 0.833 |
| TAP1 = 0.93 * GBP1 − 0.29 | 0.814 |
| TAP1 = 1.13 * ETV7 + 0.75 | 0.793 |
| TAP1 = 0.79 * CCL5 + 2.34 | 0.784 |
| TAP1 = 0.71 * CXCL10 + 2.80 | 0.779 |
| TAP1 = 1.13 * HLA_A − 6.04 | 0.778 |
| TAP1 = 1.38 * TAP2 − 2.15 | 0.772 |
| TAP1 = 1.13 * APOL3 − 0.53 | 0.769 |
| TAP1 = 1.07 * HLA_B − 5.88 | 0.765 |
| TAP1 = 1.26 * TYMP − 4.85 | 0.758 |
| TAP2 = 0.73 * TAP1 + 1.56 | 0.772 |
| TAP2 = 0.78 * STAT1 − 0.54 | 0.723 |
| TAP2 = 0.82 * HLA_A − 2.83 | 0.679 |
| TAP2 = 0.68 * GBP1 + 1.33 | 0.677 |
| TAP2 = 0.82 * CTSS + 0.19 | 0.639 |
| TAP2 = 0.82 * ETV7 + 2.11 | 0.636 |
| TBL1X = 0.91 * PRKX − 0.71 | 0.396 |
| TBL1X = 0.61 * ACTR3B + 3.67 | 0.318 |
| TBL1X = 1.21 * KEAP1 − 2.30 | 0.303 |
| TBL1Y = 1.05 * ER_067 − 0.27 | 0.850 |
| TBL1Y = 1.12 * ER_013 − 0.11 | 0.822 |
| TBL1Y = 1.09 * CALML6 − 0.41 | 0.822 |
| TBL1Y = 1.10 * ER_028 − 0.37 | 0.817 |
| TBL1Y = 1.00 * SLC22A6 − 0.25 | 0.810 |
| TBL1Y = 1.12 * IL13 − 1.01 | 0.808 |
| TBL1Y = 1.30 * DNAJB8 − 2.17 | 0.807 |
| TBL1Y = 1.12 * ER_109 + 0.20 | 0.797 |
| TBL1Y = 0.94 * DNTT + 0.08 | 0.797 |
| TBL1Y = 1.32 * ER_120 − 0.15 | 0.790 |
| TERF1 = 0.48 * RSPO2 + 6.60 | 0.712 |
| TERF1 = 0.49 * TDGF1 + 6.14 | 0.686 |
| TERF1 = 0.48 * DNAJC5B + 6.54 | 0.681 |
| TERF1 = 0.51 * INFA_Family + 4.67 | 0.678 |
| TERF1 = 0.47 * PSG2 + 6.14 | 0.659 |
| TERF1 = 0.46 * UGT2B7 + 7.13 | 0.657 |
| TGFBR2 = 0.89 * PECAM1 + 1.13 | 0.689 |
| TGFBR2 = 1.16 * ZEB2 − 0.61 | 0.659 |
| TGFBR2 = 0.65 * IL10RA + 4.11 | 0.622 |
| TGFBR2 = 1.00 * MAF − 0.52 | 0.622 |
| TGFBR2 = 0.93 * TLR4 + 2.31 | 0.618 |
| TGFBR2 = 0.89 * CSF1R + 1.92 | 0.616 |
| THBS2 = 0.92 * COL5A2 + 1.03 | 0.779 |
| THBS2 = 0.83 * COL1A2 + 0.56 | 0.766 |
| THBS2 = 0.95 * COL5A1 + 0.28 | 0.766 |
| THBS2 = 0.59 * EDIL3 + 6.48 | 0.755 |
| THBS2 = 0.66 * COL11A1 + 4.49 | 0.747 |
| THBS2 = 0.79 * COL1A1 − 1.02 | 0.746 |
| THBS2 = 0.81 * COL3A1 − 0.78 | 0.725 |
| THBS2 = 1.23 * TIMP2 − 3.44 | 0.722 |
| THBS2 = 1.03 * SPARC − 2.27 | 0.712 |
| THBS4 = 1.98 * F2R − 8.15 | 0.587 |
| THBS4 = 1.22 * SFRP4 − 4.41 | 0.573 |
| THBS4 = 1.12 * SFRP2 − 6.10 | 0.550 |
| THBS4 = 1.53 * IGF1 − 5.10 | 0.541 |
| THBS4 = 1.00 * COMP − 2.29 | 0.525 |
| THBS4 = 2.56 * ZEB1 − 15.43 | 0.516 |
| TIFA = 1.53 * NFKB1 − 5.43 | 0.497 |
| TIFA = 1.02 * MAD2L1 + 0.06 | 0.454 |
| TIFA = 0.90 * CCNA2 + 1.24 | 0.449 |
| TIMP3 = 0.65 * COMP + 5.67 | 0.680 |
| TIMP3 = 1.43 * IGFBP7 − 4.64 | 0.661 |
| TIMP3 = 1.11 * LOXL1 + 1.83 | 0.659 |
| TIMP3 = 1.02 * THBS2 + 0.56 | 0.656 |
| TIMP3 = 1.36 * PDGFRB − 0.82 | 0.638 |
| TIMP3 = 0.60 * EDIL3 + 7.16 | 0.628 |
| TK1 = 1.05 * ECT2 − 1.02 | 0.519 |
| TK1 = 0.94 * P4HB − 2.54 | 0.514 |
| TK1 = 1.08 * KPNA2 − 2.48 | 0.505 |
| TLR3 = 1.10 * CASP1 − 3.09 | 0.634 |
| TLR3 = 0.78 * GBP7 + 1.93 | 0.622 |
| TLR3 = 1.36 * IRF2 − 3.81 | 0.607 |
| TLR3 = 0.83 * GNGT2 + 2.38 | 0.595 |
| TLR3 = 0.72 * IFNG + 2.47 | 0.589 |
| TLR3 = 0.87 * IRF1 + 0.80 | 0.589 |
| TMEM45B = 1.23 * AR − 2.56 | 0.810 |
| TMEM45B = 1.01 * ABCC12 − 0.13 | 0.805 |
| TMEM45B = 1.04 * UGT1A6 − 0.29 | 0.774 |
| TMEM45B = 0.91 * ABCC11 − 0.08 | 0.773 |
| TMEM45B = 1.02 * NR0B2 + 1.40 | 0.768 |
| TMEM45B = 0.97 * TAT + 0.20 | 0.767 |
| TMEM45B = 1.07 * HMGCS2 − 1.23 | 0.764 |
| TMEM45B = 1.17 * CEACAM5 − 1.29 | 0.757 |
| TMEM45B = 1.09 * CHAD − 0.71 | 0.745 |
| TMEM45B = 1.35 * PFKFB1 − 2.70 | 0.738 |
| TMEM74B = 0.72 * TIE1 + 2.53 | 0.672 |
| TMEM74B = 0.99 * ATP7B + 0.70 | 0.662 |
| TMEM74B = 0.63 * TNNI3 + 3.50 | 0.658 |
| TMEM74B = 0.66 * JPH3 + 3.24 | 0.648 |
| TMEM74B = 0.58 * GATA4 + 4.21 | 0.635 |
| TMEM74B = 0.69 * DHH + 3.31 | 0.630 |
| TNFAIP3 = 0.63 * BIRC3 + 3.09 | 0.678 |
| TNFAIP3 = 0.53 * CCL5 + 4.05 | 0.665 |
| TNFAIP3 = 0.63 * CCL4 + 4.04 | 0.617 |
| TNFAIP3 = 0.67 * IL2RB + 3.53 | 0.614 |
| TNFAIP3 = 0.64 * IL2RG + 3.40 | 0.607 |
| TNFAIP3 = 0.78 * SOCS1 + 1.81 | 0.606 |
| TNFRSF11B = 1.02 * CCL20 + 1.81 | 0.512 |
| TNFRSF11B = 0.95 * CXCR2 + 2.39 | 0.501 |
| TNFRSF11B = 1.09 * IL7 + 0.65 | 0.493 |
| TNFRSF17 = 0.85 * CD79A + 0.77 | 0.866 |
| TNFRSF17 = 1.15 * CCR2 − 1.17 | 0.823 |
| TNFRSF17 = 1.05 * PIM2 − 2.28 | 0.810 |
| TNFRSF17 = 1.48 * BTK − 3.83 | 0.801 |
| TNFRSF17 = 0.87 * CD38 + 0.62 | 0.775 |
| TNFRSF17 = 0.70 * IRF4 + 2.04 | 0.773 |
| TNFRSF17 = 1.51 * CASP10 − 4.87 | 0.760 |
| TNFRSF17 = 1.93 * EAF2 − 7.58 | 0.756 |
| TNFRSF17 = 1.24 * TBX21 − 1.08 | 0.749 |
| TNFRSF17 = 1.51 * IL16 − 4.55 | 0.746 |
| TNFRSF8 = 0.82 * EOMES + 1.31 | 0.810 |
| TNFRSF8 = 1.24 * MFNG − 2.56 | 0.786 |
| TNFRSF8 = 0.65 * CEACAM3 + 2.83 | 0.776 |
| TNFRSF8 = 1.04 * SNAI3 − 0.11 | 0.749 |
| TNFRSF8 = 1.21 * STX11 − 2.07 | 0.739 |
| TNFRSF8 = 1.49 * PARP4 − 5.53 | 0.738 |
| TNFRSF8 = 0.63 * PLA2G3 + 3.01 | 0.733 |
| TNFRSF8 = 0.99 * TNFRSF10C − 0.49 | 0.730 |
| TNFRSF8 = 0.79 * PAX5 + 2.13 | 0.721 |
| TNFRSF8 = 0.60 * MYOD1 + 3.46 | 0.719 |
| TNFRSF9 = 0.82 * IFNG + 1.09 | 0.776 |
| TNFRSF9 = 0.92 * FASLG + 0.46 | 0.760 |
| TNFRSF9 = 0.71 * PDCD1 + 1.99 | 0.750 |
| TNFRSF9 = 0.99 * IRF1 − 0.79 | 0.743 |
| TNFRSF9 = 0.79 * ICOS + 1.35 | 0.740 |
| TNFRSF9 = 0.80 * CD274 + 0.89 | 0.739 |
| TNFRSF9 = 0.72 * GZMH + 1.79 | 0.738 |
| TNFRSF9 = 0.82 * TBX21 + 1.17 | 0.726 |
| TNFRSF9 = 1.07 * CD33 − 1.47 | 0.716 |
| TNFRSF9 = 0.86 * CXCR6 − 0.18 | 0.716 |
| TNFSF14 = 1.09 * MFNG − 1.04 | 0.809 |
| TNFSF14 = 0.94 * FASLG + 0.52 | 0.765 |
| TNFSF14 = 0.93 * XCL2 − 0.31 | 0.764 |
| TNFSF14 = 0.85 * ICOS + 1.20 | 0.759 |
| TNFSF14 = 0.77 * EOMES + 1.97 | 0.743 |
| TNFSF14 = 0.88 * TBX21 + 1.01 | 0.737 |
| TNFSF14 = 1.21 * PIK3R5 − 1.26 | 0.734 |
| TNFSF14 = 0.78 * GZMH + 1.63 | 0.734 |
| TNFSF14 = 0.85 * CCR6 + 1.44 | 0.730 |
| TNFSF14 = 0.91 * SNAI3 + 1.10 | 0.728 |
| TNXB = 1.02 * TIE1 + 0.82 | 0.803 |
| TNXB = 0.88 * IL4 + 2.08 | 0.794 |
| TNXB = 0.79 * PLA2G3 + 2.83 | 0.785 |
| TNXB = 0.90 * CECR6 + 1.86 | 0.780 |
| TNXB = 0.80 * LEP + 2.82 | 0.778 |
| TNXB = 0.83 * CMTM2 + 2.91 | 0.777 |
| TNXB = 0.92 * CIDEA + 1.63 | 0.772 |
| TNXB = 0.95 * CCL14 + 0.49 | 0.763 |
| TNXB = 0.74 * MYOD1 + 3.42 | 0.761 |
| TNXB = 1.06 * ACKR1 − 0.03 | 0.757 |
| TOP1 = 0.58 * COPS5 + 5.44 | 0.494 |
| TOP1 = −0.24 * ER_171 + 10.75 | −0.484 |
| TOP1 = 0.99 * ATP5A1 − 1.35 | 0.484 |
| TOP3A = 0.44 * IFT52 + 4.89 | 0.717 |
| TOP3A = 0.88 * POLR2D + 0.38 | 0.684 |
| TOP3A = 0.36 * ITGB7 + 5.69 | 0.659 |
| TOP3A = 0.48 * CD47 + 4.20 | 0.642 |
| TOP3A = 0.98 * PRKAB1 + 0.69 | 0.624 |
| TOP3A = 1.25 * SRSF2 − 6.38 | 0.618 |
| TSPAN13 = 1.83 * RAC1 − 12.06 | 0.490 |
| TSPAN13 = 1.15 * P4HB − 4.37 | 0.458 |
| TSPAN13 = 1.04 * RHOB − 1.89 | 0.454 |
| TSPAN7 = 1.24 * SLIT2 − 3.08 | 0.757 |
| TSPAN7 = 0.66 * CCL14 + 2.43 | 0.736 |
| TSPAN7 = 0.74 * ACKR1 + 2.07 | 0.723 |
| TSPAN7 = 0.95 * ABCA9 + 0.46 | 0.696 |
| TSPAN7 = 0.89 * IGF1 − 0.07 | 0.686 |
| TSPAN7 = 0.90 * LAMP5 + 0.94 | 0.670 |
| TTK = 1.09 * AURKB − 1.29 | 0.637 |
| TTK = 1.02 * KIF2C − 0.41 | 0.602 |
| TTK = 1.13 * CDC7 − 0.86 | 0.586 |
| TTK = 1.08 * BUB1 − 1.82 | 0.583 |
| TTK = 1.04 * NUF2 − 1.32 | 0.577 |
| TTK = 1.01 * DLGAP5 − 0.28 | 0.574 |
| UBB = 1.45 * RNF149 − 2.67 | 0.572 |
| UBB = −0.58 * STAT4 + 16.56 | −0.561 |
| UBB = −0.55 * DNAJB7 + 16.57 | −0.558 |
| UBB = −0.51 * CEACAM5 + 16.10 | −0.549 |
| UBB = −0.63 * BMP8B + 16.98 | −0.543 |
| UBB = −0.54 * TDGF1 + 16.82 | −0.542 |
| UBXN2A = 1.26 * ATRX − 4.22 | 0.393 |
| UBXN2A = 1.15 * TERF1 − 4.49 | 0.392 |
| UBXN2A = 0.57 * TDGF1 + 2.59 | 0.390 |
| UGT1A1 = 1.14 * THPO − 1.41 | 0.875 |
| UGT1A1 = 1.09 * UGT1A6 − 1.02 | 0.873 |
| UGT1A1 = 1.21 * DPPA5 − 3.16 | 0.871 |
| UGT1A1 = 1.07 * UGT1A4 − 0.72 | 0.865 |
| UGT1A1 = 1.22 * LIN28A − 1.89 | 0.863 |
| UGT1A1 = 1.13 * AQP7 − 3.72 | 0.857 |
| UGT1A1 = 1.08 * KLK3 − 0.99 | 0.852 |
| UGT1A1 = 1.05 * SLC22A7 − 0.82 | 0.850 |
| UGT1A1 = 1.18 * CXCR1 − 1.67 | 0.847 |
| UGT1A1 = 1.11 * KLK2 − 1.08 | 0.847 |
| USF2 = 0.48 * IFT52 + 4.65 | 0.693 |
| USF2 = 0.52 * CD47 + 3.90 | 0.658 |
| USF2 = 0.87 * RHOA − 1.84 | 0.645 |
| USF2 = 0.94 * FKBP8 − 1.13 | 0.618 |
| USF2 = 0.96 * POLR2D − 0.26 | 0.609 |
| USF2 = 0.63 * CEBPB + 1.23 | 0.605 |
| VCAN = 1.21 * COL1A2 − 5.23 | 0.669 |
| VCAN = 1.15 * COL1A1 − 7.53 | 0.657 |
| VCAN = 1.42 * FBN1 − 4.71 | 0.637 |
| VCAN = 1.50 * SPARC − 9.35 | 0.632 |
| VCAN = 1.36 * LOX − 2.66 | 0.620 |
| VCAN = 1.34 * MMP2 − 6.39 | 0.616 |
| VEGFB = 0.80 * PRKACA + 2.43 | 0.625 |
| VEGFB = 1.19 * ATP6V0C − 2.64 | 0.567 |
| VEGFB = 1.06 * MEN1 − 0.03 | 0.547 |
| VEGFB = 1.08 * TADA3 − 0.07 | 0.540 |
| VGLL4 = −0.72 * CASP10 + 16.67 | −0.555 |
| VGLL4 = −0.71 * IRF1 + 16.29 | −0.544 |
| VGLL4 = −0.82 * IL18 + 17.71 | −0.541 |
| VGLL4 = −0.41 * CD38 + 14.06 | −0.528 |
| VGLL4 = −0.90 * HHEX + 17.85 | −0.524 |
| VGLL4 = −0.72 * CD4 + 17.19 | −0.521 |
| VHL = 0.97 * RAF1 + 0.32 | 0.541 |
| VHL = 0.81 * CAPN7 + 2.69 | 0.515 |
| VHL = 0.80 * RUVBL1 + 2.12 | 0.463 |
| WNT10A = 0.91 * RPS6KB1 + 1.51 | 0.614 |
| WNT10A = 1.42 * RUNX3 − 4.34 | 0.603 |
| WNT10A = 0.76 * ZBTB32 + 2.88 | 0.596 |
| WNT10A = 0.71 * FGF17 + 3.22 | 0.584 |
| WNT10A = 1.04 * CCNB2 + 1.32 | 0.582 |
| WNT10A = 0.99 * ICOS + 0.15 | 0.574 |
| WNT7B = 0.89 * HOXA10 + 3.01 | 0.592 |
| WNT7B = 1.25 * ATP7B − 0.46 | 0.579 |
| WNT7B = 0.83 * JPH3 + 2.74 | 0.574 |
| WNT7B = 1.44 * KCTD11 − 2.79 | 0.567 |
| WNT7B = 0.79 * BIRC7 + 3.21 | 0.562 |
| WNT7B = 1.02 * IE11 + 0.86 | 0.555 |
| WWC1 = 0.60 * KCNIP1 + 3.10 | 0.644 |
| WWC1 = 0.59 * NPPB + 3.27 | 0.643 |
| WWC1 = 0.56 * KEK3 + 3.36 | 0.637 |
| WWC1 = 0.60 * ECN1 + 2.49 | 0.636 |
| WWC1 = 0.59 * THPO + 3.16 | 0.635 |
| WWC1 = 0.53 * PCK1 + 3.72 | 0.633 |
| WWOX = 0.51 * ER_013 + 4.67 | 0.461 |
| WWOX = 0.50 * CREB3L3 + 5.04 | 0.449 |
| WWOX = 0.51 * UTY + 5.00 | 0.433 |
| XBP1 = 0.62 * CD79A + 7.39 | 0.746 |
| XBP1 = 0.76 * PIM2 + 5.18 | 0.713 |
| XBP1 = 1.04 * BTG2 + 1.38 | 0.703 |
| XBP1 = 0.51 * IRF4 + 8.31 | 0.690 |
| XBP1 = 1.50 * HERPUD1 − 5.22 | 0.681 |
| XBP1 = 1.09 * CASP10 + 3.30 | 0.656 |
| XRCC5 = 0.96 * PMS1 + 1.34 | 0.832 |
| XRCC5 = 0.83 * MMS19 − 0.06 | 0.800 |
| XRCC5 = 0.95 * YY1 − 1.12 | 0.784 |
| XRCC5 = 1.12 * ANAPC2 − 2.68 | 0.778 |
| XRCC5 = 0.89 * ARAF − 0.90 | 0.777 |
| XRCC5 = 1.30 * SPATA2 − 2.70 | 0.775 |
| XRCC5 = 1.17 * APPBP2 − 2.47 | 0.772 |
| XRCC5 = 0.87 * ADORA2A + 1.91 | 0.767 |
| XRCC5 = 0.97 * RPTOR + 0.23 | 0.766 |
| XRCC5 = 1.09 * MCM7 + 0.54 | 0.762 |
| ZAK = −1.09 * CD33 + 15.83 | −0.473 |
| ZAK = −0.92 * IRF5 + 15.98 | −0.467 |
| ZAK = −1.54 * TEP1 + 19.96 | −0.466 |

1. A method for predicting a response or resistance to and/or a benefit from a cancer immunotherapy in a subject suffering from or being at risk of developing a neoplastic disease, optionally breast cancer, comprising:
determining in a sample obtained from said subject the expression level of at least one marker selected from the group consisting of the markers
ACKR2, ACSL3, ACSL4, ACSL5, ACTA2, ACTR3B, ADAMTS1, ADIPOR1, AGT, AHNAK, AK3, AKT2, ALDH1A3, ALDOC, ALKBH3, ANGPT1, APAF1, AR, AREG, ARID1A, ARNT, ATP5F1, ATP6V0C, ATP6V1G2, BATF, BCL10, BCL2A1, BID, BIRC7, BLM, BMP5, BOK, C5orf55, CA9, CAD, CASP8AP2, CAV1, CAV2, CBX3, CCDC103, CCL14, CCL17, CCL18, CCL19, CCL21, CCL22, CCL25, CCL28, CCL3, CCL4, CCL5, CCL7, CCND3, CCNE2, CCR4, CCT4, CCT6B, CD274, CD38, CD47, CD55, CD79A, CD83, CD86, CD8A, CDC7, CDKN2A, CDX2, CEACAM3, CEBPB, CELSR2, CHI3L1, CHMP4B, CLCF1, CMKLR1, COL1A1, COL1A2, COL2A1, COL3A1, COL5A1, COL5A2, COL9A3, COX7B, CRK, CRLF2, CRY1, CSDE1, CXCL1, CXCL10, CXCL13, CXCL16, CXCL8, CXXC4, CYP4V2, DAAM1, DDX58, DHX58, DIABLO, DLC1, DLGAP5, DLL4, DMD, DNAJA1, DNAJB2, DNAJC10, DNAJC13, DNAJC14, DNAJC8, DUSP6, E2F3, EAF2, EDIL3, EEF2K, EGFR, EIF6, ENG, EPCAM, ER154, ERBB2, ETV7, EZH2, FABP4, FADD, FAF1, FANCG, FAS, FASN, FBXO5, FBXW11, FGF13, FGF4, FGFR3, FLT3, FN1, FOSL1, GADD45G, GBP1, GBP7, GJA1, GLIS3, GMPS, GNG12, GNLY, GPAM, GPAT2, GPR17, GRIN2A, GSN, GSR, GSTM1, GZMB, HDAC8, HERPUD1, HEY2, HIC1, HIST1H3H, HLA_A, HLA_B, HLA_E, HMGB3, HMOX1, HRK, HSPA1A, HSPA1L, ID1, ID2, IDH1, IDH2, IDO1, IFI27, IFNA2, IFNA5, IFNAR1, IFNW1, IGFBP7, IL12A, IL6R, INHBA, IRF1, IRF2, IRF4, IRF7, IRF9, IRS1, ISG15, ITGA2, ITGB7, ITPKB, JAG1, JAK1, JAK2, JPH3, KCNK5, KDM1A, KDM6A, KDR, KIF3B, KNTC1, KRT18, KRT7, LAG3, LCN2, LFNG, LIF, LOX, LOXL1, LRIG1, LRP12, LYVE1, MAD2L1, MADD, MAP3K4, MAP3K5, MAPK10, MAPK3, MAT2A, MAX, MCM5, MCM6, MED12, MESP1, MGEA5, MIXL1, MLLT3, MLPH, MME, MMP14, MSH3, MSL2, MTHFD1, MX1, MYBL1, MYCN, MYOD1, NAIP, NAMPT, NASP, NCOA2, NFKB1, NKD1, NLRP3, NMU, NOD2, NOTCH1, NOTCH4, NR6A1, NRG1, NSD1, NTHL1, NTRK1, NUMBL, ORM2, P4HB, PAG1, PARP2, PAX6, PCOLCE, PDCD1LG2, PDGFB, PFKFB3, PHB, PIK3CA, PIM3, PLA2G10, PLA2G4A, PLAT, PLCB1, PLCG1, PLCG2, PLK4, PMEPA1, PML, PPARGC1A, PPID, PPP2CA, PPP2CB, PRAME, PRC1, PRDM1, PRKAA2, PRKAG1, PRKCE, PRMT6, PROM1, PRR15L, PSIP1, PSMD2, PTCHD1, PTGR1, PTP4A1, PTPN11, PTPRC, PTTG1, PYCR1, QSOX2, RAB6B, RAC3, RAD51C, RAD9A, RARB, RASSF1, RB1, RBP1, RELN, RIPK3, RPL13, RPL6, RUNX1, S100A6, SCUBE2, SELE, SERPINB2, SERPINF1, SETD2, SFRP2, SFRP4, SHC2, SLAMF7, SLC11A1, SLC16A1, SLC16A2, SLC25A13, SLC45A3, SLIT2, SMAD2, SMC1A, SMC4, SNCA, SOCS4, SORT1, SPARC, SPDEF, SPINK1, SPOP, SPRY2, SPRY4, SRF, SRM, STAT1, STEAP4, STK3, STK39, STX1A, TADA3, TAP1, TAP2, TBL1X, TBL1Y, TERF1, TGFBR2, THBS2, THBS4, TIFA, TIMP3, TK1, TLR3, TMEM45B, TMEM74B, TNFAIP3, TNFRSF11B, TNFRSF17, TNFRSF8, TNFRSF9, TNFSF14, TNXB, TOP1, TOP3A, TSPAN13, TSPAN7, TTK, UBB, UBXN2A, UGT1A1, USF2, VCAN, VEGFB, VGLL4, VHL, WNT10A, WNT7B, WWC1, WWOX, XBP1, XRCC5, ZAK CASP4, LRRK2, GGH, C3AR1, ARMC1, FANCC, MAF, RASA1, PIAS1, HERC3, SLA, CFLAR, RUNX2, FAF1, CTLA4, TNFSF14, MAPKAPK5, LAMAS, PTEN, BID, FYN, E2F3, ALDH1A1, PDPN, NOX4, MYBL2, RBP1, SYCP2
wherein the expression level of the at least one marker is indicative for predicting the response or resistance to and/or the benefit from the treatment with the cancer immunotherapy in said subject.

2. A method for predicting the outcome of a cancer immunotherapy in a subject suffering from or being at risk of developing a neoplastic disease, optionally breast cancer, comprising:
determining in a sample obtained from said subject the expression level of at least one marker selected from the group consisting of the markers
ACKR2, ACSL3, ACSL4, ACSL5, ACTA2, ACTR3B, ADAMTS1, ADIPOR1, AGT, AHNAK, AK3, AKT2, ALDH1A3, ALDOC, ALKBH3, ANGPT1, APAF1, AR, AREG, ARID1A, ARNT, ATP5F1, ATP6VOC, ATP6V1G2, BATF, BCL10, BCL2A1, BID, BIRC7, BLM, BMP5, BOK, C5orf55, CA9, CAD, CASP8AP2, CAV1, CAV2, CBX3, CCDC103, CCL14, CCL17, CCL18, CCL19, CCL21, CCL22, CCL25, CCL28, CCL3, CCL4, CCL5, CCL7, CCND3, CCNE2, CCR4, CCT4, CCT6B, CD274, CD38, CD47, CD55, CD79A, CD83, CD86, CD8A, CDCl₇, CDKN2A, CDX2, CEACAM3, CEBPB, CELSR2, CHI3L1, CHMP4B, CLCF1, CMKLR1, COL1A1, COL1A2, COL2A1, COL3A1, COL5A1, COL5A2, COL9A3, COX7B, CRK, CRLF2, CRY1, CSDE1, CXCL1, CXCL10, CXCL13, CXCL16, CXCL8, CXXC4, CYP4V2, DAAM1, DDX58, DHX58, DIABLO, DLC1, DLGAP5, DLL4, DMD, DNAJA1, DNAJB2, DNAJC10, DNAJC13, DNAJC14, DNAJC8, DUSP6, E2F3, EAF2, EDIL3, EEF2K, EGFR, EIF6, ENG, EPCAM, ER_154, ERBB2, ETV7, EZH2, FABP4, FADD, FAF1, FANCG, FAS, FASN, FBXO5, FBXW11, FGF13, FGF4, FGFR3, FLT3, FN1, FOSL1, GADD45G, GBP1, GBP7, GJA1, GLIS3, GMPS, GNG12, GNLY, GPAM, GPAT2, GPR17, GRIN2A, GSN, GSR, GSTM1, GZMB, HDAC8, HERPUD1, HEY2, HIC1, HIST1H3H, HLA_A, HLA_B, HLA_E, HMGB3, HMOX1, HRK, HSPA1A, HSPA1L, ID1, ID2, IDH1, IDH2, IDO1, IFI27, IFNA2, IFNA5, IFNAR1, IFNW1, IGFBP7, IL12A, IL6R, INHBA, IRF1, IRF2, IRF4, IRF7, IRF9, IRS1, ISG15, ITGA2, ITGB7, ITPKB, JAG1, JAK1, JAK2, JPH3, KCNK5, KDM1A, KDM6A, KDR, KIF3B, KNTC1, KRT18, KRT7, LAG3, LCN2, LFNG, LIF, LOX, LOXL1, LRIG1, LRP12, LYVE1, MAD2L1, MADD, MAP3K4, MAP3K5, MAPK10, MAPK3, MAT2A, MAX, MCM5, MCM6, MED12, MESP1, MGEA5, MIXL1, MLLT3, MLPH, MME, MMP14, MSH3, MSL2, MTHFD1, MX1, MYBL1, MYCN, MYOD1, NAIP, NAMPT, NASP, NCOA2, NFKB1, NKD1, NLRP3, NMU, NOD2, NOTCH1, NOTCH4, NR6A1, NRG1, NSD1, NTHL1, NTRK1, NUMBL, ORM2, P4HB, PAG1, PARP2, PAX6, PCOLCE, PDCD1LG2, PDGFB, PFKFB3, PHB, PIK3CA, PIM3, PLA2G10, PLA2G4A, PLAT, PLCB1, PLCG1, PLCG2, PLK4, PMEPA1, PML, PPARGC1A, PPID, PPP2CA, PPP2CB, PRAME, PRC1, PRDM1, PRKAA2, PRKAG1, PRKCE, PRMT6, PROM1, PRR15L, PSIP1, PSMD2, PTCHD1, PTGR1, PTP4A1, PTPN11, PTPRC, PTTG1, PYCR1, QSOX2, RAB6B, RAC3, RAD51C, RAD9A, RARB, RASSF1, RB1, RBP1, RELN, RIPK3, RPL13, RPL6, RUNX1, S100A6, SCUBE2, SELE, SERPINB2, SERPINF1, SETD2, SFRP2, SFRP4, SHC2, SLAMF7, SLC11A1, SLC16A1, SLC16A2, SLC25A13, SLC45A3, SLIT2, SMAD2, SMC1A, SMC4, SNCA, SOCS4, SORT1, SPARC, SPDEF, SPINK1, SPOP, SPRY2, SPRY4, SRF, SRM, STAT1, STEAP4, STK3, STK39, STX1A, TADA3, TAP1, TAP2, TBL1X, TBL1Y, TERF1, TGFBR2, THBS2, THBS4, TIFA, TIMP3, TK1, TLR3, TMEM45B, TMEM74B, TNFAIP3, TNFRSF11B, TNFRSF17, TNFRSF8, TNFRSF9, TNFSF14, TNXB, TOP1, TOP3A, TSPAN13, TSPAN7, TTK, UBB, UBXN2A, UGT1A1, USF2, VCAN, VEGFB, VGLL4, VHL, WNT10A, WNT7B, WWC1, WWOX, XBP1, XRCC5, ZAK
CASP4, LRRK2, GGH, C3AR1, ARMC1, FANCC, MAF, RASA1, PIAS1, HERC3, SLA, CFLAR, RUNX2, FAF1, CTLA4, TNFSF14, MAPKAPK5, LAMAS, PTEN, BID, FYN, E2F3, ALDH1A1, PDPN, NOX4, MYBL2, RBP1, SYCP2
wherein the expression level of the at least one marker is indicative for the outcome in said subject.

3. The method of claim 1, wherein the expression level of at least one marker selected from the group consisting of PTPN11, DIABLO, PARP2, MTHFD1, MAX, HERPUD1, RAD51C, P4HB, PYCR1, SPOP, PHB, XRCC5, PPP2CB, MYBL1, STK3, TNFRSF17, CD79A, COL9A3, PLA2G4A, SPRY2, KCNK5, DMD, DDX58, ISG15, IF127, MX1, IRF9, IRF7, CXCL1, CXCL8, CCL19, CCL7, LAG3, THBS4, PTPRC, ITGB7, PRDM1, TNFRSF9, CD86, CXCL13, CXCL16, STAT1, IDO1, GBP1, IRF1, TAP1, CXCL10, KRT7, KRT18, DLGAP5, MCM6, FBXO5, E2F3, EZH2, FANCG, TTK, KDM1A, MCM5, GMPS, NASP, SMC4, MAD2L1, KNTC1, PRC1, CDCl₇, TK1, CCNE2, BLM, COL3A1, MMP14, SFRP2, COL5A1, COL1A2, COL1A1, FN1, LOXL1, PCOLCE, COL5A2, SPARC, IGFBP7, THBS2, SFRP4, VCAN, CD38, GNLY, GZMB, SLAMF7, CD8A, IRF4 and CCL5,
optionally DDX58, LAG3, THBS4, COL3A1, COL1A1, CD38, GNLY, IF127, MX1, IRF9, IRF7, CXCL13, STAT1, GBP1, IRF1, TAP1, CXCL10, KDM1A, KNTC1, SPARC, IGFBP7, SLAMF7, RAD51C, P4HB, MYBL1, PLA2G4A, CCL19, CCL7, KRT7, MMP14, SFRP2, COL5A1 and COL1A2,
optionally DDX58, LAG3, THBS4, COL3A1, COL1A1, CD38 and GNLY is determined.

4. The method of claim 1, wherein the expression level of at least one marker related to immune response and/or a marker related to antigen-presentation of a tumor cell is determined.

5. The method of claim 4, wherein the at least one marker related to immune response is selected from the group consisting of CCL19, CCL7, LAG3, THBS4, PTPRC, ITGB7, PRDM1, TNFRSF9, CD86, CXCL13 and CXCL16, optionally CCL19, CCL7, LAG3, THBS4, TNFRSF9, CD86 and CXCL13, optionally CCL19, CCL7, LAG3, THBS4 and CXCL13.

6. The method of claim 4, wherein the at least one marker related to antigen-presentation of a tumor cell is selected from the group consisting of CD38, GNLY, GZMB, SLAMF7, CD8A, IRF4 and CCL5, optionally said maker is GNLY or GZMB.

7. The method of claim 1, wherein the expression level of at least one marker selected from the group consisting of the markers
ACSL4, AKT2, BCL2A1, BLM, BTK, CA9, CASP8AP2, CCL5, CCL7, CCNA2, CCR2, CD27, CD274, CD38, CD79A, CD83, CDKN2A, CXCL10, CXCL13, CXCR3, CYBB, CYP3A4, DDX58, DHX58, DLGAP5, DMD, DNAJB7, DNAJC14, ETV7, FGF14, FGL2, GBP1, GNLY, GSTA2, GZMB, HERPUD1, HIST1H3H, HLA_A, HLA_B, HLA_E, IFIT2, IFNA2, IFNA5, IL10RA, IL12A, IL17F, IL2, IL2RB, IL2RG, IL6R, IRF2, IRF4, IRF7, IRF9, ISG15, JAK2, KDM1A, KNTC1, LAG3, MAD2L1, MAPK10, MCM6, MLLT3, MSL2, MTHFD1, MX1, OAS1, PDCD1LG2, PIM2, PLK4, PML, PRF1, PSIP1, RAB6B, RSPO2, SCN3A, SLAMF7, SLC22A2, SOCS4, SRM, STAT1, TAP1, TAP2, TBL1X, TIFA, TLR3, TNFRSF17
ACKR1, ACTA2, ACTB, AHNAK, BATF, BCL10, BMP5, BOK, CALML6, CAV1, CAV2, CCL14, CCL17, CD55, CHMP4B, CLCF1, CMKLR1, COL11A1, COL1A1, COL1A2, COL3A1, COL5A1, COL5A2, CRY1, DLL4, DNAJB14, DNAJB2, DNAJB8, EDIL3, EGFR, ENG, ER_013, ER_028, ER_067, FBN1, FGF13, FN1, GSN, GSR, HEY2, HIC1, HSPA9, IGFBP7, IL13, INHBA, IRS1, ITGA2, JAG1, KDR, LFNG, LOX, LRP12, MED12, MFNG, MMP2, MMS19, NOTCH1, NOTCH4, PAG1, PDGFB, PIM3, PLAT, PMEPA1, PPP2CB, PRKCE, PRMT6, RAC3, RB1, RIPK3, RUNX1, S100A6, SERPINF1, SFRP2, SHC2, SLC22A6, SLC25A13, SLIT2, SNCA, SPARC, SPRY4, SRF, STK3, STK39, TBL1Y, THBS2, THBS4, TIE1, TIMP2, TIMP3, TMEM74B, TNFRSF11B, TNFSF14, TNXB, TRIB1, VEGFB, YY1
ACSL4, ACTR3B, ADRA1D, AGT, AK3, AKT2, ALDOC, BCL2A1, CA9, CCDC103, CCL25, CCL3, CCL5, CD47, CEBPB, CHGA, CHI3L1, DDX58, DHX58, EAF2, ER_013, ER_028, ER_109, ER_154, ERBB2, FGF8, GATA1, GBP1, GJA1, GNLY, GRIN2A, GZMB, HAND1, HDAC8, HLA_A, HLA_B, HLA_E, HNF1B, HSPA1L, ID2, IDH1, IFT52, IL2RB, IL6R, IRF2, ISG15, ITPKB, JAK2, LAG3, LRIG1, MADD, MAX, MLLT3, MX1, MYBL1, NFE2L2, NFKB1, NTRK1, ORM2, PFKFB3, PLA2G4A, PPID, PRF1, PSIP1, PTP4A1, PTPN5, QSOX2, RARB, SLC11A1, SLC16A1, SLC3A1, SOCS4, SPOP, STAT1, TAP1, TAP2, TERF1, TLR3, TNFAIP3, TNFRSF10C, TOP3A, UBB, VCAN, WNT7A, WWOX
ACTB, ADAMTS1, ADIPOR1, ALKBH3, ATP5F1, BID, CAD, CCL17, CCL28, CCT4, COL1A1, COL1A2, COL3A1, COL5A1, COL5A2, CRLF2, CXCL8, DIABLO, DNAJB14, EIF6, EOMES, FASN, FBN1, FGFR3, FN1, GPAT2, GSN, HEY2, HRK, HSPA9, KDR, KRT7, LCN2, LOX, MED12, MMP14, MMP2, MMS19, NKD1, NLRP3, NOD2, NSD1, NUMBL, P4HB, PIK3CA, PMS1, PRKAA2, PTPN11, RAD51C, RUNX1, SELE, SERPINF1, SFRP2, SLC16A2, SLC45A3, SPARC, SPRY2, STK3, TADA3, THBS4, TIE1, TIMP3, TK1, TMEM74B, TNFRSF8, TNXB, TOP1, TRIB1, TSPAN13, XRCC5, YY1
is determined.

8. The method of claim 1, wherein the neoplastic disease is a recurrent neoplastic disease or a metastatic neoplastic disease or a non-metastatic disease.

9. The method of claim 1, wherein the neoplastic disease is a disease selected form the group consisting of breast cancer, lung cancer, renal cell carcinoma, melanoma, bladder cancer, urothelial carcinoma and Merkel-cell carcinoma, optionally breast cancer.

10. The method of claim 1, wherein the cancer immunotherapy is selected from the group consisting of immune checkpoint inhibitor therapy, chimeric antigen receptor (CAR) T-Cell therapy and cancer vaccine therapy, optionally immune checkpoint inhibitor therapy.

11. The method of claim 10, wherein the immune checkpoint inhibitor is selected from the group consisting of a drug targeting CTLA4, a drug targeting PD-1 and a drug targeting PD-L1, optionally an anti-CTLA4 antibody, an anti-PD-1 antibody or an anti-PD-L1 antibody, optionally the immune checkpoint inhibitor is selected from the group consisting of ipilimumab, nivolumab, pembrolizumab, atezolizumab, avelumab, durvalumab, cemiplimab, lambrolizumab, pidilizumab or a combination thereof.

12. The method of claim 1, wherein the prediction of the response, resistance, benefit and/or outcome is for a combination of the cancer immunotherapy with a non-chemotherapy and/or a chemotherapy, optionally a neoadjuvant therapy.

13. The method of claim 1, wherein the prediction of the response, resistance, benefit and/or outcome is for a combination of the cancer immunotherapy with a chemotherapy, optionally a neoadjuvant therapy.

14. The method of claim 13, wherein the chemotherapy comprises one or more of the chemotherapeutic agent(s) selected from the group consisting of paclitaxel and nab-paclitaxel, optionally nab-paclitaxel.

15. The method of claim 1, wherein the response, resistance, benefit and/or outcome is the pathological complete response (pCR), loco-regional recurrence free interval (LRRFI), loco-regional invasive recurrence free interval (LRIRFI), distant-disease-free survival (DDFS), invasive disease-free survival (IDFS), event free survival (EFS) and/or overall survival (OS).

16. The method of claim 1, wherein the method comprises comparing the expression level of each of said at least one marker to a predetermined reference level, optionally the reference level comprises expression level of the at least one marker in a sample obtained from at least one healthy subject, optionally mean expression level of the at least one marker in samples obtained from a healthy population.

17. The method of claim 1, wherein the method further comprises determination of one or more clinical parameters selected from the group consisting of pathological grading of the tumor, tumor size and nodal status.

18. The method of claim 1, wherein in said sample obtained from said subject the expression levels of at least two, at least three, at least four, at least five, at least ten, at least twenty markers selected from the group consisting of the markers
ACSL4, AK3, AKT2, BCL2A1, CA9, CCL5, CD47, DDX58, DHX58, EAF2, GBP1, GNLY, GZMB, HLA_A, HLA_B, HLA_E, IFT52, IL2RB, IL6R, IRF2, ISG15, JAK2, LAG3, MADD, MLLT3, MX1, NFKB1, PRF1, PSIP1, SOCS4, STAT1, TAP1, TAP2, TERF1, TLR3
ER_013, ER_028
ACTB, ATP5F1, BID, CCL17, COL1A1, COL1A2, COL3A1, COL5A1, COL5A2, CXCL8, DNAJB14, FASN, FBN1, FN1, GSN, HEY2, HSPA9, KDR, LOX, MED12, MMP2, MMS19, NUMBL, P4HB, RUNX1, SERPINF1, SFRP2, SPARC, STK3, THBS4, TIE1, TIMP3, TMEM74B, TNXB, TOP1, TRIB1, YY1
ACSL4, AKT2, BCL2A1, BLM, BTK, CA9, CASP8AP2, CCL5, CCL7, CCNA2, CCR2, CD27, CD274, CD38, CD79A, CD83, CDKN2A, CXCL10, CXCL13, CXCR3, CYBB, CYP3A4, DDX58, DHX58, DLGAP5, DMD, DNAJB7, DNAJC14, ETV7, FGF14, FGL2, GBP1, GNLY, GSTA2, GZMB, HERPUD1, HIST1H3H, HLA_A, HLA_B, HLA_E, IFIT2, IFNA2, IFNA5, IL10RA, IL12A, IL17F, IL2, IL2RB, IL2RG, IL6R, IRF2, IRF4, IRF7, IRF9, ISG15, JAK2, KDM1A, KNTC1, LAG3, MAD2L1, MAPK10, MCM6, MLLT3, MSL2, MTHFD1, MX1, OAST, PDCD1LG2, PIM2, PLK4, PML, PRF1, PSIP1, RAB6B, RSPO2, SCN3A, SLAMF7, SLC22A2, SOCS4, SRM, STAT1, TAP1, TAP2, TBL1X, TIFA, TLR3, TNFRSF17
ACKR1, ACTA2, ACTB, AHNAK, BATF, BCL10, BMP5, BOK, CALML6, CAV1, CAV2, CCL14, CCL17, CD55, CHMP4B, CLCF1, CMKLR1, COL11A1, COL1A1, COL1A2, COL3A1, COL5A1, COL5A2, CRY1, DLL4, DNAJB14, DNAJB2, DNAJB8, EDIL3, EGFR, ENG, ER_013, ER_028, ER_067, FBN1, FGF13, FN1, GSN, GSR, HEY2, HIC1, HSPA9, IGFBP7, IL13, INHBA, IRS1, ITGA2, JAG1, KDR, LFNG, LOX, LRP12, MED12, MFNG, MMP2, MMS19, NOTCH1, NOTCH4, PAG1, PDGFB, PIM3, PLAT, PMEPA1, PPP2CB, PRKCE, PRMT6, RAC3, RB1, RIPK3, RUNX1, S100A6, SERPINF1, SFRP2, SHC2, SLC22A6, SLC25A13, SLIT2, SNCA, SPARC, SPRY4, SRF, STK3, STK39, TBL1Y, THBS2, THBS4, TIE1, TIMP2, TIMP3, TMEM74B, TNFRSF11B, TNFSF14, TNXB, TRIB1, VEGFB, YY1
ACSL4, ACTR3B, ADRA1D, AGT, AK3, AKT2, ALDOC, BCL2A1, CA9, CCDC103, CCL25, CCL3, CCL5, CD47, CEBPB, CHGA, CHI3L1, DDX58, DHX58, EAF2, ER_013, ER_028, ER_109, ER_154, ERBB2, FGF8, GATA1, GBP1, GJA1, GNLY, GRIN2A, GZMB, HAND1, HDAC8, HLA_A, HLA_B, HLA_E, HNF1B, HSPA1L, ID2, IDH1, IFT52, IL2RB, IL6R, IRF2, ISG15, ITPKB, JAK2, LAG3, LRIG1, MADD, MAX, MLLT3, MX1, MYBL1, NFE2L2, NFKB1, NTRK1, ORM2, PFKFB3, PLA2G4A, PPID, PRF1, PSIP1, PTP4A1, PTPN5, QSOX2, RARB, SLC11A1, SLC16A1, SLC3A1, SOCS4, SPOP, STAT1, TAP1, TAP2, TERF1, TLR3, TNFAIP3, TNFRSF10C, TOP3A, UBB, VCAN, WNT7A, WWOX
ACTB, ADAMTS1, ADIPOR1, ALKBH3, ATP5F1, BID, CAD, CCL17, CCL28, CCT4, COL1A1, COL1A2, COL3A1, COL5A1, COL5A2, CRLF2, CXCL8, DIABLO, DNAJB14, EIF6, EOMES, FASN, FBN1, FGFR3, FN1, GPAT2, GSN, HEY2, HRK, HSPA9, KDR, KRT7, LCN2, LOX, MED12, MMP14, MMP2, MMS19, NKD1, NLRP3, NOD2, NSD1, NUMBL, P4HB, PIK3CA, PMS1, PRKAA2, PTPN11, RAD51C, RUNX1, SELE, SERPINF1, SFRP2, SLC16A2, SLC45A3, SPARC, SPRY2, STK3, TADA3, THBS4, TIE1, TIMP3, TK1, TMEM74B, TNFRSF8, TNXB, TOP1, TRIB1, TSPAN13, XRCC5, YY1
CASP4, LRRK2, GGH, C3AR1, ARMC1, FANCC, MAF, RASA1, PIAS1, HERC3, SLA, CFLAR, RUNX2, FAF1, CTLA4, TNFSF14, MAPKAPK5, LAMA5, PTEN, BID, FYN, E2F3, ALDH1A1, PDPN, NOX4, MYBL2, RBP1, SYCP2
are determined.

19. The method of claim 17, comprising determining a score based on
(i) expression levels of the at least two, at least three, at least four, at least five, at least ten, at least twenty markers; or
(ii) expression level of the at least one marker and the at least one clinical parameter.

20. The method of claim 1,
(a) wherein the at least one marker is selected from the group of
the markers as identified in Table 2.1, optionally in Table 2.2, optionally in Table 2.3, (optionally) in Table 2.4, optionally in Table 2.5, (optionally) in Table 2.6, optionally in Table 2.7, (optionally) in Table 2.8, optionally in Table 2.9, optionally in Table 2.10, optionally in Table 2.11 and optionally in Table 2.12; and/or
(b) wherein the at least one marker is selected from the group of the markers as identified in Table 3.1, optionally in Table 3.2, optionally in Table 3.3, optionally in Table 3.4, optionally in Table 3.5, optionally in Table 3.6, optionally in Table 3.7, optionally in Table 3.8, optionally in Table 3.9, optionally in Table 3.10, optionally in Table 3.11 and optionally in Table 3.12; and/or
(c) wherein the at least one marker is selected from the group of the markers as identified in Table 4.1, optionally in Table 4.2, optionally in Table 4.3, optionally in Table 4.4, optionally in Table 4.5, optionally in Table 4.6, optionally in Table 4.7, optionally in Table 4.8, optionally in Table 4.9, optionally in Table 4.10, optionally in Table 4.11 and optionally in Table 4.12; and/or
(d) wherein the at least one marker is selected from the group of the markers as identified in Table 5.1, optionally in Table 5.2, optionally in Table 5.3, optionally in Table 5.4, optionally in Table 5.5, optionally in Table 5.6, optionally in Table 5.7, optionally in Table 5.8, optionally in Table 5.9, optionally in Table 5.10, optionally in Table 5.11 and optionally in Table 5.12; and/or
(e) wherein the at least one marker is selected from the group of the markers as identified in Table 6.1, optionally in Table 6.2, optionally in Table 6.3, optionally in Table 6.4, mom optionally in Table 6.5, optionally in Table 6.6, optionally in Table 6.7, optionally in Table 6.8, optionally in Table 6.9, optionally in Table 6.10, optionally in Table 6.11 and optionally in Table 6.12; and/or
(f) wherein the at least one marker is selected from the group of
the markers as identified in Table 7; and/or
(g) wherein the at least one marker is selected from the group of the markers as identified in Table 8.1, optionally in Table 8.2, optionally in Table 8.3, optionally in Table 8.4, optionally in Table 8.5, optionally in Table 8.6, optionally in Table 8.7, optionally in Table 8.8, optionally in Table 8.9, optionally in Table 8.10, optionally in Table 8.11 and optionally in Table 8.12.

21. Cancer immunotherapy for treatment of a neoplastic disease, wherein the cancer immunotherapy treatment is adapted to be administered to a subject that has been identified to respond to said treatment or that has been identified to benefit from said treatment or for whom said treatment has been determined to have a positive outcome according to any of the method according to claim 1.
